(19) United States
(12) Patent Application Publication
(10) Pub. No.: US 2011/0070266 A1
(43) Pub. Date: Mar. 24, 2011

(54) IMMUNOGENIC COMPOSITIONS FOR CHLAMYDIA TRACHOMATIS

(76) Inventors: Guido Grandi, Milano (IT); Giulio Ratti, Siena (IT); Alessandra Bonci, Siena (IT); Oretta Finco, Castelnuovo Berardenga (IT)

(21) Appl. No.: 12/896,353

(22) Filed: Oct. 1, 2010

Related U.S. Application Data
(62) Division of application No. 11/018,868, filed on Dec. 22, 2004, now abandoned.
Application No. 11/018,868 is a continuation-in-part of application No. PCT/US2004/020491, filed on Jun. 25, 2004.
(60) Provisional application No. 60/576,375, filed on Jun. 1, 2004.
(60) Provisional application No. 60/497,649, filed on Aug. 25, 2003.

(30) Foreign Application Priority Data
Jun. 26, 2003 (GB) 0315020.8
Feb. 2, 2004 (GB) 0402236.4

(51) Int. Cl. A61K 39/118; A61P 31/04

(52) U.S. Cl. 424/263.1

(57) ABSTRACT

The invention relates to immunogenic compositions comprising combinations of Chlamydia trachomatis antigens and their use in vaccines. The composition may comprise at least two components, one component of which comprises Chlamydia trachomatis antigens for eliciting a Chlamydia trachomatis specific TH1 immune response and another component of which comprises antigens for eliciting a Chlamydia trachomatis specific TH2 immune response. The invention further relates to an immunogenic composition comprising a Chlamydia trachomatis Type III secretion system (TTSS) regulatory protein and a Chlamydia trachomatis Type III secretion system (TTSS) secreted protein or a fragment thereof. The invention further relates to the use of combinations of adjuvants for use with antigens associated with a sexually transmissible disease, such as Chlamydia trachomatis antigens. Preferred adjuvant combinations include mineral salts, such as aluminium salts and oligonucleotides comprising a CpG motif. The invention further provides a combination of Chlamydia trachomatis antigens comprising a Chlamydia trachomatis antigen that is conserved over at least two serovars.

This application is a division of Ser. No. 11/018,868 filed on Dec. 22, 2004, which is a continuation-in-part application of PCT/US2004/020491 filed on Jun. 25, 2004, which claims priority to UK application no. 0315020.8 filed on Jun. 26, 2003; U.S. Ser. No. 60/497,649 filed on Aug. 25, 2003; UK application no. 0402236.4 filed on Feb. 2, 2004; and U.S. Ser. No. 60/576,375 filed on Jun. 1, 2004, each of which is incorporated by reference herein in its entirety.

This application incorporates by reference a 327 kb text file created on Oct. 1, 2010 and named “DIV11018868sequencelisting.txt,” which is the sequence listing for this application.

### FIELD OF THE INVENTION

This invention is in the fields of immunology and vaccinology. In particular, it relates to antigens derived from Chlamydia trachomatis and their use in immunization.

### BACKGROUND OF THE INVENTION

The Chlamydiae are obligate intracellular parasites of eukaryotic cells which are responsible for endemic sexually transmitted infections and various other disease syndromes. They occupy an exclusive eubacterial phylogenic branch, having no close relationship to any other known organisms.

Historically, the Clamydiae have been classified in their own order (Chlamydiales) made up of a single family (Chlamydiaceae) which in turn contains a single genus (Chlamydia, also referred to as Chlamydophila). More recently, this order has been divided into at least four families including Chlamydiaceae, Parachlamydiaceae, Waddiaceae and Simkaniaceae. In this more recent classification, the Chlamydiaceae family includes genuses of Chlamydophila and Chlamydia, Chlamydia trachomatis being a species within the Chlamydia genus. See, Bush et al., (2001) Int. J. Syst. Evol. Microbiol. 51:203-220.

A particular characteristic of the Chlamydiae is their unique life cycle, in which the bacterium alternates between two morphologically distinct forms: an extracellular infective form (elementary bodies, EB) and an intracellular non-infective form (reticulate bodies, RB). The life cycle is completed with the re-organization of RB into EB, which leave the disrupted host cell ready to infect further cells.

The genome sequences of at least five chlamydia or chlamydophila species are currently known—C. trachomatis, C. pneumoniae, C. muridarum, C. pecorum and C. psittaci (See Kalman et al., (1999) Nature Genetics 21:385-389; Read et al. (2000) Nucleic Acids Res. 28:1397-1406; Shirai et al. (2000) Nucleic Acids Res 28:2311-2314; Stephens et al. (1998) Science 282:754-759; and International patent publications WO99/27105, WO00/27994 and WO99/28475).

The human serovariants (“serovars”) of C. trachomatis are divided into two biovariants (“biovars”). Serovars A-K elicit epithelial infections primarily in the ocular tissue (A-C) or urogenital tract (D-K). Serovars L1, L2 and L3 are the agents of invasive lymphogranuloma venereum (LGV). Recently, researchers in the field have demonstrated that there are very low differences between all the genomes of Chlamydia trachomatis. It appears that the three distinct tissue tropisms for strains of Chlamydia trachomatis (i.e. ocular, urogenital and lymph node) are due to relatively few changes between the coding regions of the different strains (see Brunelle et al (2004) Genome Biology 5: R42).

Although chlamydial infection itself causes disease, it is thought that the severity of symptoms in some patients is actually due to an aberrant host immune response. Failure to clear the infection results in persistent immune stimulation and, rather than helping the host, this results in chronic infection with severe consequences, including sterility and blindness. See, e.g., Ward, (1995) Apmis. 103:769-96. In addition, the protection conferred by natural chlamydial infection is usually incomplete, transient, and strain-specific.

More than 4 million new cases of chlamydial sexually transmitted infections are diagnosed each year in the United States alone and the cost of their treatment has been estimated in 4 billion dollars annually, with 80% attributed to infection and disease of women. Although chlamydial infections can be treated with several antibiotics, a majority of the female infections are asymptomatic, and antimicrobial therapy may be delayed or inadequate to prevent long term sequelae, especially in countries with poor hygienic conditions. Multiple-antibiotic-resistant strains of Chlamydia have also been reported (Somani, et al., 2000). Furthermore it has been suggested that antibiotic treatment could lead to the formation of aberrant forms of C. trachomatis that maybe reactivated later on (See, Hammerschlag M. R., (2002) Semin. Pediatr. Infect. Dis. 13:239-248).

Unfortunately the major determinants of chlamydial pathogenesis are complicated and at present still unclear, mostly due to the intrinsic difficulty in working with this pathogen and the lack of adequate methods for its genetic manipulation. In particular very little is known about the antigenic composition of elementary body surface, that is an essential compartment in pathogen-host interactions, and likely to carry antigens able to elicit a protective immune response.

Due to the serious nature of the disease, there is a desire to provide suitable vaccines. These may be useful (a) for immunization against chlamydial infection or against chlamydia-induced disease (prophylactic vaccination) or (b) for the eradication of an established chronic chlamydial infection (therapeutic vaccination). Being an intracellular parasite, however, the bacterium can generally evade antibody-mediated immune responses.

Various antigenic proteins have been described for C. trachomatis, and the cell surface in particular has been the target of detailed research. See, e.g., Moulder (1991) Microbiol Rev 55(1):143-190. These include, for instance, Pgp3, MOMP, Hsp60 (GroEL) and Hsp70 (DnaK like). References describing Pgp3 include Comanducci et al. (1994) Infect Immun 62(12):5491-5497 and patent publications EP 0499681 and WO95/28487). References describing MOMP include Murdin et al. (1993) Infect Immun 61:4406-4414. References describing Hsp60 (GroEL) include Cerrone et al. (1991) Infect Immun 59(1):79-90). References describing Hsp70 (DnaK-like) include Raulston et al. (1993) J. Biol. Chem. 268:23139-23147). Not all of these have proved to be effective vaccines, however, and further candidates have been identified. See WO03/049762.

Vaccines against pathogens such as hepatitis B virus, diphtheria and tetanus typically contain a single protein antigen (e.g. the HBV surface antigen, or a tetanus toxoid). In contrast, acellular whooping cough vaccines typically have at least three B. pertussis proteins, and the Prevnar™ pneumococcal vaccine contains seven separate conjugated saccharide antigens. Other vaccines such as cellular pertussis vaccines, the measles vaccine, the inactivated polio vaccine (IPV) and meningococcal OMV vaccines are by their very nature complex mixtures of a large number of antigens. Whether protection can be elicited by a single antigen, a small number of defined antigens, or a complex mixture of undefined antigens, therefore depends on the pathogen in question.

It is an object of the invention to provide further and improved compositions for providing immunity against chlamydial disease and/or infection. In particular, it is an object of the invention to provide a composition capable of eliciting both a humoral immune response and a cell mediated immune response that are required for protection against Chlamydia infection and/or for elimination of existing Chlamydia infection. In addition, it is an object to provide a composition that elicits a protective immune response against multiple Chlamydial serovars.

The compositions of the present invention are based on a combination of two or more (e.g. three or more) C. trachomatis antigens. In addition, the compositions may also be based on the use of C. trachomatis antigens with a combination of adjuvants designed to elicit an enhanced immune response. Preferably, the combination of adjuvants comprises an aluminium salt and an oligonucleotide comprising a CpG motif.

### SUMMARY OF THE INVENTION

Within the ˜900 proteins previously described for the C. trachomatis genome (See e.g., Stephens et al. (1998) Science 282:754-759), Applicants have discovered a group of Chlamydia trachomatis antigens that are particularly suitable for immunization purposes, particularly when used in combinations.

In one aspect of the present invention, an immunogenic composition is provided comprising a combination of at least one antigen that elicits a Chlamydia trachomatis specific TH1 immune response (such as a cell mediated or cellular immune response) and at least one antigen that elicits a Chlamydia trachomatis specific TH2 response (such as a humoral or antibody response). The immunogenic composition may further comprise a TH1 adjuvant and a TH2 adjuvant.

In another aspect of the present invention, an immunogenic composition is provided comprising a combination of Chlamydia trachomatis antigens comprising at least one Chlamydia trachomatis antigen that is conserved over at least two serovars.

In yet another aspect of the present invention, an immunogenic composition is provided comprising a combination of at least one antigen that elicits a Chlamydia trachomatis specific TH1 immune response and at least one antigen that elicits a Chlamydia trachomatis specific TH2 immune response, the combination comprising at least one Chlamydia trachomatis antigen that is conserved over at least two serovars. In one embodiment, the at least two serovars are selected from the group consisting of serovars D, E, F, G, H, I, J, and K.

In another aspect of the present invention, the immunogenic composition comprising at least one antigen that elicits a Chlamydia trachomatis specific TH1 immune response and at least one antigen that elicits a Chlamydia trachomatis specific TH2 immune response preferably comprises a combination of Chlamydia trachomatis antigens comprising at least one Chlamydia trachomatis antigen associated with the EB of Chlamydia trachomatis and at least one Chlamydia trachomatis antigen associated with the RB of Chlamydia trachomatis. Still further such combinations can comprise EB and/or RB antigens from one serovar combined with RB and/or EB antigens from at least one other serovar.

In an additional aspect of the present invention, a kit is provided comprising a combination of Chlamydia trachomatis antigens wherein at least one of the Chlamydia trachomatis antigens is associated with the EB of Chlamydia trachomatis and at least one of the Chlamydia trachomatis antigens is associated with the RB of Chlamydia trachomatis. The kit may further include a TH1 adjuvant, a TH2 adjuvant and instructions.

The present invention further provides methods of eliciting a Chlamydia specific immune response by administering an immunogenic composition of this invention.

The present invention further provides a method of monitoring the efficacy of treatment of a subject infected with Chlamydia trachomatis comprising determining the level of Chlamydia specific antibody or Chlamydia specific effector moleculein the subject after administration of an immunogenic composition of this invention.

### BRIEF DESCRIPTION OF THE DRAWINGS

FIG. 1 depicts a western blot analysis of total protein extracts from C. trachomatis EBs, performed using mouse immune sera against recombinant antigens. Only FACS positive non neutralizing sera are shown. For antigen identification, please see Table 1(a). The panel identification numbers correspond to the numbers reported in the WB analysis column of Table 1(a). In each panel, the strip on the right shows the results obtained with the antigen-specific immune serum (I), and the strip on the left shows the results obtained with the corresponding preimmune serum (P).

FIG. 2 illustrates serum titers giving 50% neutralization of infectivity for the 9 C. trachomatis recombinant antigens described in the text (PepA, ArtJ, DnaK, CT398, CT547, Enolase, MOMP, OmpH-like and AtoS. Each titre was assessed in 3 separate experiments (SEM values shown).

FIG. 3 includes FACS analysis of antibody binding to whole C. trachomatis EBs. Gray histograms (event counts versus fluorescence channels) are the FACS output for EBs stained with background control antibodies. White histograms are the FACS output of EBs stained with antigen-specific antibodies. Positive control was represented by an anti-C. trachomatis mouse hyperimmune serum against whole EBs, with the corresponding preimmune mouse serum as background control; Negative controls were obtained by staining EBs with either mouse anti-GST or mouse anti-HIS hyperimmune serum, with the corresponding preimmune serum as background control. For each serum the background control was represented by mouse anti-GST or mouse anti-HIS hyperimmune serum, depending on the fusion protein used for immunization. Western blotting data obtained from total EB proteins stained with the same antiserum used for the FACS assays are also shown within each panel.

### DETAILED DESCRIPTION OF THE INVENTION

As noted above, C. trachomatis may be classified according to their serological reactivities with polyclonal or monoclonal antisera (i.e., “serovars”). These serological differences are typically detected due to differences in the MOMP (Major Outer Membrane Protein) of C. trachomatis. There are currently at least 18 serovars of C. trachomatis, including the D, E, F, G, H, I, J, and K serovars that are typically associated with genital tract disease.

In particular, Serovars D, E, F, H and K account for nearly 85% of genital tract infections (see for example, WO 02/065129). Research to date also indicates that the 4 Serovars (or serotypes) responsible for Sexually Transmitted Infections or Diseases (STIs or STDs) in the US and Europe are D, E, F and I. Other biotypes of C. trachomatis include serovars A, B, Ba, and C which are associated with trachoma, a transmissible condition of the eye or L1, L2 and L3 which are associated with Lymphogranuloma venereum (LGV) which is a sexually transmitted systemic infection. LGV is uncommon in industralised countries but frequent in Africa, Asia, Australian and South America. It predominantly affects lymphatic tissue but may also occur as an acute symptomatic infection without apparent lymph node involvement or tissue reaction at the point of infection. Acute LGV is reported over five times more frequent in men than in women.

The present invention provides a Chlamydia trachomatis antigen comprising an amino acid sequence that is conserved across more than one Chlamydia trachomatis serovar. For example, a C. trachomatis antigen is provided that comprises an amino acid sequence that is conserved across at least 2 of serovars D, E, F, G, H, I, J, and K. Alternatively, the C. trachomatis antigen may comprise an amino acid sequence that is conserved across a first serovar selected from the group consisting of serovar D, E, F, G, H, I, J, and K and a second sevovar selected from the group consisting of A, B, Ba, C, L1, L2 and L3. The C. trachomatis antigen of the present invention may also comprise an amino acid sequence that is conserved across any 2 or more serovars.

The present invention also provides a combination of C. trachomatis antigens comprising at least one C. trachomatis antigen associated with a first serovar and at least one C. trachomatis antigen associated with a second serovar, the second serovar being different from the first serovar. For example, the combination of C. trachomatis antigens may comprise a first C. trachomatis antigen associated with one of serovars D, E, F, G, H, I, J, and K and a second C. trachomatis antigen associated with another one of serovars D, E, F, G, H, I, J, and K. As an example, the first C. trachomatis antigen is associated with serovar D and the second C. trachomatis antigen is associated with serovar E or the first C. trachomatis antigen is associated with serovar D and the second C. trachomatis antigen is associated with serovar F or the first C. trachomatis antigen is associated with serovar D and the second C. trachomatis antigen is associated with serovar G or the first C. trachomatis antigen is associated with serovar D and the second C. trachomatis antigen is associated with serovar H or the first C. trachomatis antigen is associated with serovar D and the second C. trachomatis antigen is associated with serovar I or the first C. trachomatis antigen is associated with serovar D and the second C. trachomatis antigen is associated with serovar J or the first C. trachomatis antigen is associated with serovar D and the second C. trachomatis antigen is associated with serovar D or the first C. trachomatis antigen is associated with serovar E and the second C. trachomatis antigen is associated with serovar F or the first C. trachomatis antigen is associated with serovar E and the second C. trachomatis antigen is associated with serovar G or the first C. trachomatis antigen is associated with serovar E and the second C. trachomatis antigen is associated with serovar H or the first C. trachomatis antigen is associated with serovar E and the second C. trachomatis antigen is associated with serovar I or the first C. trachomatis antigen is associated with serovar E and the second C. trachomatis antigen is associated with serovar J or the first C. trachomatis antigen is associated with serovar E and the second C. trachomatis antigen is associated with serovar K or the first C. trachomatis antigen is associated with serovar F and the second C. trachomatis antigen is associated with serovar G or the first C. trachomatis antigen is associated with serovar F and the second C. trachomatis antigen is associated with serovar H or the first C. trachomatis antigen is associated with serovar F and the second C. trachomatis antigen is associated with serovar I or the first C. trachomatis antigen is associated with serovar F and the second C. trachomatis antigen is associated with serovar J or the first C. trachomatis antigen is associated with serovar F and the second C. trachomatis antigen is associated with serovar K or the first C. trachomatis antigen is associated with serovar G and the second C. trachomatis antigen is associated with serovar H or the first C. trachomatis antigen is associated with serovar G and the second C. trachomatis antigen is associated with serovar I or the first C. trachomatis antigen is associated with serovar G and the second C. trachomatis antigen is associated with serovar J or the first C. trachomatis antigen is associated with serovar G and the second C. trachomatis antigen is associated with serovar K or the first C. trachomatis antigen is associated with serovar H and the second C. trachomatis antigen is associated with serovar I or the first C. trachomatis antigen is associated with serovar H and the second C. trachomatis antigen is associated with serovar J or the first C. trachomatis antigen is associated with serovar H and the second C. trachomatis antigen is associated with serovar K or the first C. trachomatis antigen is associated with serovar I and the second C. trachomatis antigen is associated with serovar J or the first C. trachomatis antigen is associated with serovar I and the second C. trachomatis antigen is associated with serovar K or the first C. trachomatis antigen is associated with serovar J and the second C. trachomatis antigen is associated with serovar K.

Alternatively, the first C. trachomatis antigen is associated with any one of serovars D-K and the second C. trachomatis antigen is associated with any serovar other than serovars D-K. Also, both the first and the second C. trachomatis antigens may be associated with serovars other than serovars D-K.

Preferably, the present invention provides an immunogenic composition comprising a combination of C. trachomatis antigens associated with any one of serovars D, E, F and I.

The present invention also provides a combination of C. trachomatis antigens comprising at least one C. trachomatis antigen comprising an amino acid sequence that is conserved across 2 or more serovars of C. trachomatis. For example, the combination of C. trachomatis antigens may comprise at least one C. trachomatis antigen comprising an amino acid sequence that is conserved across at least 2 serovars selected from the group consisting of serovars D, E, F, G, H, I, J and K.

As discussed above, the invention further provides compositions comprising a combination of Chlamydia trachomatis antigens, wherein the combinations can be selected from groups of antigens which Applicants have identified as being particularly suitable for immunization purposes, particularly when used in combination. In particular, the combination of Chlamydia trachomatis antigens may comprise at least two components—component A and component B, wherein each component comprises Chlamydia trachomatis antigens expressed or secreted at different stages of the C. trachomatis life cycle.

Chlamydia trachomatis exhibits a unique biphasic life cycle in which the organism transitions between an infectious, extracellular elementary body (EB) and an intracellular reticulate body (RB). EBs attach to and enter host cells. After entry into host cells, chlamydiae EBs remain within a membrane-bound vacuole, termed an inclusion, which avoids fusion with host lysosomes, and where the EBs differentiate into the larger metabolically active reticulate body form (RBs) and multiply intracellularly in the RB form. RBs also produce proteins intracellularly that may be, for example, released into the cytosol of host cells, broken down in the cytosol of host cells, or presented to the host cell surface. Transition of Chlamydia trachomatis from EB to RB occurs within the first several hours of infection. RBs continue to multiply in the host cells and produce proteins associated with the RB intracellularly until late in the infection cycle when RBs transition back to EBs. The host cells may then lyse, thus releasing mature EBs which re-infect other host cells. The extracellular EB is generally regarded as the only infectious Chlamydia form because it is exposed to the immune system. Thus, the EB proteome has been viewed as a source of effective anti-Chlamydial vaccine targets so that antibody present in the genital tract or ocular secretions—arising from a humoral or Th2 response—may inhibit infection during the extracellular EB stage of the Chlamydia trachomatis life cycle. The RB, on the other hand, resides within an intracellular inclusion and thus, is not accessible to antibodies present in the genital tract or ocular secretions. Accordingly, the resolution of infection at the RB stage may require a cell-mediated immune response mediated by Th1 cells. Thus an ideal immunogenic composition or vaccine to protect against Chlamydia trachomatis infection is capable of inducing both antibody (at least immunoglobulin G (IgG)) responses in mucosal secretions and at mucosal surfaces to prevent infection by Chlamydial EB and/or a strong Th1 response to limit ascending infection to the uterus and fallopian tubes.

The present invention further provides a combination of Chlamydia trachomatis antigens comprising at least a component A and a component B. Component A comprises at least one Chlamydia trachomatis antigen for eliciting at least a Chlamydia trachomatis specific TH2 immune response and component B comprises at least one Chlamydia trachomatis antigen for eliciting at least a Chlamydia trachomatis specific TH1 immune response. As an example, component A of the combination of Chlamydia trachomatis antigens may include at least one antigen expressed on or by elementary bodies (EBs) of Chlamydia trachomatis and component B of the combination of Chlamydia trachomatis antigens may include at least one antigen expressed or secreted by or translocated into or by the reticulate bodies (RBs) of Chlamydia trachomatis.

Differentiation, isolation and identification of antigens or proteins associated with C. trachomatis EBs and antigens or proteins associated with C. trachomatis RBs may be accomplished in a variety of ways. For example, density gradient ultra centrifugation methods may be used as disclosed in WO 02/082091, incorporated herein in its entirety. EBs of C. trachomatis measure approximately 300 nm in diameter and have a condensed nucleus. RBs of C. trachomatis measure approximately 1000 nm and have a normal bacterial nucleus. Thus, EBs and RBs may be effectively separated by, for example, density gradient ultra centrifugation methods to purify EBs and RBs as well as intermediate forms of C. trachomatis. Purity can further be determined by electron microscopy or verified on silverstained 2D gels. The purity of EB and RB preparations may also be confirmed using MOMP, Hc1- and IncG-specific antibodies (see Fields and Hackstadt (2000) Molecular Microbiology 38(5) 1048-1060). By way of example, MOMP is found in both chlamydial developmental forms (Hatch et al (1984) J Bacteriol 157: 13-20; Hackstadt et al (1985) J Bacteriol 161: 25-31) whereas the histone-like protein Hc1 is detected only in EBs (Hackstadt et al (1991) PNAS 88: 3937-3941) and the inclusion membrane protein IncG is detected in RBs but not in EBs (Scidmore-Carlson et al (1999) Mol Microbiol 33: 753-765).

WO 02/082091 discloses one example of identifying secreted proteins from an intracellular bacteria such as C. trachomatis in which host cells are infected by C. trachomatis which are labeled in the infected cells. Protein profiles from 2D gel electrophoresis of whole cell lysates of the infected cells are compared with protein profiles from purified and lysed bacteria from the infected cells. If protein spots of whole cell lysates of the infected cells are absent (or in significantly reduced amounts) in the purified bacteria, the proteins corresponding to such spots are determined to be secreted from the intracellular bacteria (i.e., RB of C. trachomatis).

A further example of identifying secreted proteins from an intracellular bacteria such as C. trachomatis is disclosed in WO 02/082091 in which host cells are infected by intracellular bacteria and are cultivated in the presence and in the absence of a proteasome inhibitor. The intracellular bacteria (e.g., C. trachomatis) is labeled in the respective infected host cells and whole cell lysates of the infected cells are prepared. 2D-gel electrophoresis protein profiles of whole cell lysates of the infected cells with proteasome inhibitor is compared to that of whole cell lysates of the infected cells without proteasome inhibitor. If protein spots of the whole cell lysates of the infected cells without proteasome inhibitor is absent or significantly reduced as compared with the whole cell lysates of the infected cells with proteasome inhibitor, the corresponding protein may be identified as a protein secreted from an intracellular bacteria (e.g., C. trachomatis).

Also, the sub-cellular localization of antigens may be accomplished through polyclonal antibodies as disclosed in WO 02/082091. In this alternative method, proteins of intracellular bacteria are identified and antibodies to the identified proteins are obtained. Protein spots are identified that react on 2D-PAGE immunoblotting on whole cell lysates of cells infected with the bacteria using the obtained antibodies.

EB associated antigens may also be identified via identification of surface exposed proteins. For example, antibodies to C. trachomatis antigens may be analyzed for their capability to recognize surface exposed proteins on purified EBs, as determined by a FACS K-S binding assay. Proteins showing a K-S score higher than 8.0 are listed as FACS positive. Proteins that are FACS positive are deemed to be likely EB associated antigens. To increase specificity of the analysis of EB and RB antigens, analysis of antiserum to recombinant antigens may be accomplished by Western blot in which whole protein extracts of purified chlamydial EBs are screened by Western blot analysis. Sera that recognizes a band of expected molecular weight on Ells protein extracts are deemed to be “consistent.” If there is a presence of a band at the expected molecular mass plus several additional bands of weaker intensity, the sera is deemed “partially consistent.” Sera that gives a negative Western blot pattern are deemed “non consistent” and form bands that do not correspond to the expected molecular weight. In one method of identifying EB or RB antigens, a FACS K-S score of greater than 10.0 and a Western Blot analysis of either “consistent” or “partially consistent” indicate an EB antigen of C. trachomatis.

In another method of identifying EB and RB antigens of C. trachomatis, antisera is evaluated for in vitro neutralizing properties. For example, infectious EBs may be incubated with sera from mice immunized with a C. trachomatis recombinant antigen of interest and then tested for their capability to infect a monolayer of epithelial cells.

The inhibition of infectivity due to EBs interaction with immune sera is calculated as the percentage reduction in Chlamydia inclusion number. Sera are considered neutralizing if they cause a 50% or greater reduction in infectivity. A neutralizing serum titre above 1:300 is deemed “high”. A neutralizing serum titre in a range of from about 1:180 and 1:300 is deemed an “intermediate” neutralizing titre and a neutralizing serum titre equal or less than 1:100 is deemed a “low” titre. Thus, in this method of identifying EB and RB antigens of C. trachomatis, a FACS K-S score greater than 10 and either a “consistent” or “partially consistent” result in the Western Blot or a neutralizing titre of greater than 150 indicates the antigen is associated with the EB form of C. trachomatis.

RB antigens may be identified by their association with the inclusion membrane of C. trachomatis. As described above, the intracellular form of C. trachomatis resides in the host cell within a vacuole surrounded by an inclusion membrane. C. trachomatis secretes proteins into the inclusion membrane. Component B of the present invention may include an RB associated protein that is localized to the inclusion membrane, such as, for example, IncG.

Identification of such RB associated proteins in cells or tissues may be accomplished in a variety of ways. For example, fixed layers of C. trachomatis infected cells may be reacted with anti-sera to a C. trachomatis antigen of interest. Using immunofluorescence microscopy indirect immunofluorescence and immunochemistry, RB associated proteins may then be localized within the infected cells or tissues. For example, IncA protein is localized to the inclusion membrane using immunofluorescence microscopy as described (Bannantine, et al., Infection and Immunity, December 1998, p. 6017-6021, incorporated herein in its entirety).

In another embodiment, component A of the combination may include at least one Type III Secretion System (TTSS) protein and component B of the combination may include at least one Type III Secretion System (TTSS) secreted or effector protein or fragment thereof. There are many methods for identifying TTSS proteins (i.e., TTSS proteins associated with the Chlamydial TTSS machinery). TTSS is a complex protein secretion and delivery machine or apparatus, which may be located, either wholly or partially, on the Elementary Body (EB) and which allows an organism, such as Chlamydia, to maintain its intracellular niche by injecting proteins, such as bacterial effector proteins (which may act as anti-host virulence determinants) into the cytosol of a eukaryotic cell in order to establish the bacterial infection and to modulate the host cellular functions. TTSS proteins exposed on the EB surface may play a role in adhesion and/or uptake into host cells. There are at least 12 TTSS proteins exposed on the EB surface of C. trachomatis that are known in the art. Identification of such TTSS proteins may be accomplished, for example, by raising antibodies against TTSS proteins from sera from infected animals and screening for reactivity to TTSS components assembled and exposed on the EB surface. Also, TTSS proteins associated with the EB of C. trachomatis may be detected in purified EBs using MALDI-TOF (Matrix-Assisted-Laser-Desorption-Ionization-Time-of-Flight) and immunoblot analyses or detected by electron microscopy as rod-like projections on the surface of the bacteria (Fields, K. A., Mead, K J., Dooley, C A., and T. Hackstadt. (2003) Chlamydia trachomatis type III secretion: evidence for a functional apparatus during early-cycle development. Mol Micro 48:671; Chang, J. J., Leonard, K. R., and Y. X., Mang. (1997) Structural studies of the surface projections of Chlamydia trachomatis by electron microscopy. J Med Micro 46:1013, 66(12); incorporated herein in their entireties).

Each of component A and component B is described in more detail below.

### Component A

In one embodiment, the invention provides component A of a composition comprising a combination of Chlamydia trachomatis antigens that elicit at least a TH2 immune response. For example, component A of the combination of the present invention may comprise at least one C. trachomatis EB antigen. As an example, component A of the combination of the present invention may consist of two, three, four or all five Chlamydia trachomatis antigens of a first antigen group, said first antigen group consisting of: (1) PepA (CT045); (2) LcrE (CT089); (3) ArtJ (CT381); (4) DnaK (CT396); and (5) CT398. These antigens are referred to herein as the ‘first antigen group’.

Preferably, component A of the composition of the invention comprises a combination of Chlamydia trachomatis antigens, said combination selected from the group consisting of: (1) PepA & LcrE; (2) PepA & ArtJ; (3) PepA & DnaK; (4) PepA & CT398; (5) LcrE & ArtJ; (6) LcrE & DnaK; (7) LcrE & CT398; (8) ArtJ & DnaK; (9) ArtJ & CT398; (10) DnaK & CT398; (11) PepA, LcrE & ArtJ; (12) PepA, LcrE & DnaK; (13) PepA, LcrE & CT398; (14) PepA, ArtJ & DnaK; (15) PepA, ArtJ and CT398; (16) PepA, DnaK & CT398; (17) LcrE, ArtJ & DnaK; (18) LcrE, ArtJ & CT398; (19) LcrE, DnaK & CT398; (20) ArtJ, DnaK & CT398; (21) PepA, LcrE, ArtJ & DnaK; (22) PepA, LcrE, DnaK & CT398; (23) PepA, ArtJ, DnaK & CT398; (24) PepA, LcrE, ArtJ & CT398; (25) LcrE, ArtJ, DnaK & CT398; and (26) PepA, LcrE, ArtJ, DnaK & CT398. Preferably, the composition of Chlamydia trachomatis antigens consists of PepA, LcrE, ArtJ, DnaK & CT398. Preferably, the combination includes LcrE (CT089).

The invention also provides for a slightly larger group of Chlamydia trachomatis antigens for component A of 13 Chlamydia trachomatis antigens that are particularly suitable for immunization purposes, particularly when used in combinations. (This second antigen group includes the five Chlamydia trachomatis antigens of the first antigen group.) These 13 Chlamydia trachomatis antigens form a second antigen group of (1) PepA (CT045); (2) LcrE (CT089); (3) ArtJ (CT381); (4) DnaK (CT396); (5) CT398; (6) OmpH-like (CT242); (7) L7/L12 (CT316); (8) OmcA (CT444); (9) AtoS (CT467); (10) CT547; (11) Eno (CT587); (12) HtrA (CT823) and (13) MurG (CT761). These antigens are referred to herein as the ‘second antigen group’.

The invention therefore provides component A of a composition comprising a combination of Chlamydia trachomatis antigens, said combination selected from the group consisting of two, three, four, five, six, seven, eight, nine, ten, eleven, twelve, or thirteen Chlamydia trachomatis antigens of the second antigen group. Preferably, the combination is selected from the group consisting of two, three, four or five Chlamydia trachomatis antigens of the second antigen group. Still more preferably, component A of the combination consists of five Chlamydia trachomatis antigens of the second antigen group. Preferably, component A of the combination includes one or both of LcrE (CT089) and OmpH-like protein (CT242).

Each of the Chlamydia trachomatis antigens of the first and second antigen group are described in more detail below.

(1) PepA leucyl aminopeptidase A protein (CT045) One example of a ‘PepA’ protein is disclosed as SEQ ID NOs: 71 & 72 in WO 03/049762 (GenBank accession number: AAC67636, GI:3328437; ‘CT045’; SEQ ID NO: 1 in attached sequence listing). It is believed to catalyse the removal of unsubstituted N-terminal amino acids from various polypeptides. Preferred PepA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1; and/or (b) which is a fragment of at least n consecutive amino acids of SEQ ID NO: 1, wherein n is 7 or more (e.g. 8, 10, 12, 14, 16, 18, 20, 25, 30, 35, 40, 50, 60, 70, 80, 90, 100, 150, 200, 250 or more). These PepA proteins include variants (e.g. allelic variants, homologs, orthologs, paralogs, mutants, etc.) of SEQ ID NO: 1. Preferred fragments of (b) comprise an epitope from SEQ ID NO: 1. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 1. Other fragments omit one or more domains of the protein (e.g. omission of a signal peptide, of a cytoplasmic domain, of a transmembrane domain, or of an extracellular domain). The PepA protein may contain manganese ions.

(2) LcrE low calcium response E protein (CT089) One example of a ‘LcrE’ protein is disclosed as SEQ ID NOs: 61 & 62 in WO 03/049762 (GenBank accession number: AAC67680, GI:3328485; ‘CT089’; SEQ ID NO: 2 in attached sequence listing). Preferred LcrE proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2; and/or (b) which is a fragment of at least n consecutive amino acids of SEQ ID NO: 2, wherein n is 7 or more (e.g. 8, 10, 12, 14, 16, 18, 20, 25, 30, 35, 40, 50, 60, 70, 80, 90, 100, 150, 200, 250 or more). These LcrE proteins include variants (e.g. allelic variants, homologs, orthologs, paralogs, mutants, etc.) of SEQ ID NO: 2. Preferred fragments of (b) comprise an epitope from SEQ ID NO: 2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 2. Other fragments omit one or more domains of the protein (e.g. omission of a signal peptide, of a cytoplasmic domain, of a transmembrane domain, or of an extracellular domain). CT089, also known as CopN, is the chlamydial homolog of YopN, a protein that is secreted by the Type III secretion system of Yersinia. CopN is thought to act as a peripherally associated regulator that prevents secretion in the absence of proper signals from the host cell: in effect, it is thought to “plug” the terminal end of the secretion apparatus until an inductive signal is received. As the Examples and our earlier studies demonstrate (see Montigiani et al (2002) Infection and Immunity 70: 368-379), CT089 (LcrE) appears to be present and accessible to antibodies on the surface of the infectious EB form which makes this protein a good component of an immunogenic composition since the efficient blocking of the Type Three Secretion (TTS) system/organelle/apparatus may in turn inhibit or at least down-regulate the Chlamydia infection process by “freezing” the LcrE negative regulator.

(3) ArtJ arginine-binding protein (CT381) One example of ‘ArtJ’ protein is disclosed as SEQ ID NOs: 105 & 106 in WO 03/049762 (GenBank accession number: AAC67977, GI:3328806; ‘CT381’; SEQ ID NO: 3 in attached sequence listing). Preferred ArtJ proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 3; and/or (b) which is a fragment of at least n consecutive amino acids of SEQ ID NO: 3, wherein n is 7 or more (e.g. 8, 10, 12, 14, 16, 18, 20, 25, 30, 35, 40, 50, 60, 70, 80, 90, 100, 150, 200, 250 or more). These ArtJ proteins include variants (e.g. allelic variants, homologs, orthologs, paralogs, mutants, etc.) of SEQ ID NO: 3. Preferred fragments of (b) comprise an epitope from SEQ ID NO: 3. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 3. Other fragments omit one or more domains of the protein (e.g. omission of a signal peptide, of a cytoplasmic domain, of a transmembrane domain, or of an extracellular domain). The ArtJ protein may be bound to a small molecule like arginine or another amino acid.

(4) DnaK heat-shock protein 70 (chaperone) (CT396) One example of ‘DnaK’ protein is disclosed as SEQ ID NOs: 107 & 108 in WO 03/049762 (GenBank accession number: AAC67993, GI:3328822; ‘CT396’; SEQ ID NO: 4 in attached sequence listing). Other sequences are disclosed in Birkelund et al. (1990) Infect Immun 58:2098-2104; Danilition et al. (1990) Infect Immun 58:189-196; and Raulston et al. (1993) J Biol Chem 268:23139-23147. Preferred DnaK proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4; and/or (b) which is a fragment of at least n consecutive amino acids of SEQ ID NO: 4, wherein n is 7 or more (e.g. 8, 10, 12, 14, 16, 18, 20, 25, 30, 35, 40, 50, 60, 70, 80, 90, 100, 150, 200, 250 or more). These DnaK proteins include variants (e.g. allelic variants, homologs, orthologs, paralogs, mutants, etc.) of SEQ ID NO: 4. Preferred fragments of (b) comprise an epitope from SEQ ID NO: 4. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 4. Other fragments omit one or more domains of the protein (e.g. omission of a signal peptide, of a cytoplasmic domain, of a transmembrane domain, or of an extracellular domain). The DnaK may be phosphorylated e.g. at a threonine or a tyrosine.

(5) CT398 protein (Hypothetical Protein) One example of ‘CT398’ protein is disclosed as SEQ ID NOs: 111 & 112 in WO 03/049762 (GenBank accession number: AAC67995, GI:3328825; SEQ ID NO: 5 in attached sequence listing). Preferred CT398 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 5; and/or (b) which is a fragment of at least n consecutive amino acids of SEQ ID NO: 5, wherein n is 7 or more (e.g. 8, 10, 12, 14, 16, 18, 20, 25, 30, 35, 40, 50, 60, 70, 80, 90, 100, 150, 200, 250 or more). These CT398 proteins include variants (e.g. allelic variants, homologs, orthologs, paralogs, mutants, etc.) of SEQ ID NO: 5. Preferred fragments of (b) comprise an epitope from SEQ ID NO: 5. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 5. Other fragments omit one or more domains of the protein (e.g. omission of a signal peptide, of a cytoplasmic domain, of a transmembrane domain, or of an extracellular domain).

(6) OmpH-like outer membrane protein (CT242) One example of ‘OmpH-like’ protein is disclosed as SEQ ID NOs: 57 & 58 in WO 03/049762 (GenBank accession number: AAC67835, GI:3328652; ‘CT242’; SEQ ID NO: 6 in attached sequence listing). A variant sequence is disclosed in Bannantine & Rockey (1999) Microbiology 145:2077-2085. Preferred OmpH-like proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) which is a fragment of at least n consecutive amino acids of SEQ ID NO: 6, wherein n is 7 or more (e.g. 8, 10, 12, 14, 16, 18, 20, 25, 30, 35, 40, 50, 60, 70, 80, 90, 100, 150, 200, 250 or more). These OmpH-like proteins include variants (e.g. allelic variants, homologs, orthologs, paralogs, mutants, etc.) of SEQ ID NO: 6. Preferred fragments of (b) comprise an epitope from SEQ ID NO: 6. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more; preferably 19 or more, to remove the signal peptide) from the N-terminus of SEQ ID NO: 6. Other fragments omit one or more domains of the protein (e.g. omission of a signal peptide as described above, of a cytoplasmic domain, of a transmembrane domain, or of an extracellular domain). WO 99/53948 and Bannantine and Rockey (1999) Microbiology 145: 2077-2085 teach that CT242 is an Inclusion Membrane Associated protein in the sense that it is localised to Chlamydia intracellular developmental forms at the margins of growing inclusions.

(7) L7/L12 ribosomal protein (CT316) One example of ‘L7/L12’ protein is deposited in GenBank under accession number AAC67909 (GI:3328733; ‘CT316’; SEQ ID NO: 7 in attached sequence listing). Preferred L7/L12 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) which is a fragment of at least n consecutive amino acids of SEQ ID NO: 7, wherein n is 7 or more (e.g. 8, 10, 12, 14, 16, 18, 20, 25, 30, 35, 40, 50, 60, 70, 80, 90, 100, 150, 200, 250 or more). These L7/L12 proteins include variants (e.g. allelic variants, homologs, orthologs, paralogs, mutants, etc.) of SEQ ID NO: 7. Preferred fragments of (b) comprise an epitope from SEQ ID NO: 7. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 7. Other fragments omit one or more domains of the protein (e.g. omission of a signal peptide, of a cytoplasmic domain, of a transmembrane domain, or of an extracellular domain). The L7/L12 protein may be N-terminally modified. Although CT316 (L7/L12) protein is a ribosomal protein located within the EB, we have shown that anti-CT316 antibodies can be detected in sera from subjects with a Chlamydia infection (see for example, WO 00/37494). Thus the existence of a Chlamydia protein in an EB does not preclude the surface exposure of the Chlamydia protein at some stage in the Chlamydial developmental cycle.

(8) OmcA cysteine-rich lipoprotein (CT444) One example of ‘OmcA’ protein is disclosed as SEQ ID NOs: 127 & 128 in WO 03/049762 (GenBank accession number: AAC68043, GI:3328876; ‘CT444’, ‘Omp2A’, ‘Omp3’; SEQ ID NO: 8 in attached sequence listing). A variant sequence is disclosed in Allen et al. (1990) Mol. Microbiol. 4:1543-1550. Preferred OmcA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) which is a fragment of at least n consecutive amino acids of SEQ ID NO: 8, wherein n is 7 or more (e.g. 8, 10, 12, 14, 16, 18, 20, 25, 30, 35, 40, 50, 60, 70, 80, 90, 100, 150, 200, 250 or more). These OmcA proteins include variants (e.g. allelic variants, homologs, orthologs, paralogs, mutants, etc.) of SEQ ID NO: 8. Preferred fragments of (b) comprise an epitope from SEQ ID NO: 8. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more; preferably 18 or more to remove the signal peptide) from the N-terminus of SEQ ID NO: 8. Other fragments omit one or more domains of the protein (e.g. omission of a signal peptide as described above, of a cytoplasmic domain, of a transmembrane domain, or of an extracellular domain). The protein may be lipidated (e.g. by a N-acyl diglyceride), and may thus have a N-terminal cysteine.

(9) AtoS two-component regulatory system sensor histidine kinase protein (CT467) One example of ‘AtoS’ protein is disclosed as SEQ ID NOs: 129 & 130 in WO 03/049762 (GenBank accession number: AAC68067, GI:3328901; ‘CT467’; SEQ ID NO: 9 in attached sequence listing). Preferred AtoS proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 9; and/or (b) which is a fragment of at least n consecutive amino acids of SEQ ID NO: 9, wherein n is 7 or more (e.g. 8, 10, 12, 14, 16, 18, 20, 25, 30, 35, 40, 50, 60, 70, 80, 90, 100, 150, 200, 250 or more). These AtoS proteins include variants (e.g. allelic variants, homologs, orthologs, paralogs, mutants, etc.) of SEQ ID NO: 9. Preferred fragments of (b) comprise an epitope from SEQ ID NO: 9. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 9. Other fragments omit one or more domains of the protein (e.g. omission of a signal peptide, of a cytoplasmic domain, of a transmembrane domain, or of an extracellular domain). We have demonstrated that CT467 and its Chlamydia pneumoniae (Cpn) counterpart (Cpn0584) are neutralizing for their own species but they are also cross-protective.

(10) CT547 protein (Hypothetical Protein) One example of ‘CT547’ protein is disclosed as SEQ ID NOs: 151 & 152 in WO 03/049762 (GenBank accession number: AAC67995, GI:3328825; SEQ ID NO: 10 in attached sequence listing). Preferred CT547 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) which is a fragment of at least n consecutive amino acids of SEQ ID NO: 10, wherein n is 7 or more (e.g. 8, 10, 12, 14, 16, 18, 20, 25, 30, 35, 40, 50, 60, 70, 80, 90, 100, 150, 200, 250 or more). These CT547 proteins include variants (e.g. allelic variants, homologs, orthologs, paralogs, mutants, etc.) of SEQ ID NO: 10. Preferred fragments of (b) comprise an epitope from SEQ ID NO: 10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 10. Other fragments omit one or more domains of the protein (e.g. omission of a signal peptide, of a cytoplasmic domain, of a transmembrane domain, or of an extracellular domain).

(11) Enolase (2-phosphoglycerate dehydratase) protein (CT587) One example of an ‘Eno’ protein is disclosed as SEQ ID NOs: 189 & 190 in WO 03/049762 (GenBank accession number: AAC68189, GI:3329030; ‘CT587’; SEQ ID NO: 11 in attached sequence listing). Preferred Eno proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) which is a fragment of at least n consecutive amino acids of SEQ ID NO: 11, wherein n is 7 or more (e.g. 8, 10, 12, 14, 16, 18, 20, 25, 30, 35, 40, 50, 60, 70, 80, 90, 100, 150, 200, 250 or more). These Eno proteins include variants (e.g. allelic variants, homologs, orthologs, paralogs, mutants, etc.) of SEQ ID NO: 11. Preferred fragments of (b) comprise an epitope from SEQ ID NO: 11. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 11. Other fragments omit one or more domains of the protein (e.g. omission of a signal peptide, of a cytoplasmic domain, of a transmembrane domain, or of an extracellular domain). The Eno protein may contain magnesium ions, and may be in the form of a homodimer.

(12) HrtA DO protease protein (CT823) One example of an ‘HrtA’ protein is disclosed as SEQ ID NOs: 229 & 230 in WO 03/049762 (GenBank accession number: AAC68420, GI:3329293; ‘CT823’; SEQ ID NO: 12 in attached sequence listing). Preferred HrtA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) which is a fragment of at least n consecutive amino acids of SEQ ID NO: 12, wherein n is 7 or more (e.g. 8, 10, 12, 14, 16, 18, 20, 25, 30, 35, 40, 50, 60, 70, 80, 90, 100, 150, 200, 250 or more). These HrtA proteins include variants (e.g. allelic variants, homologs, orthologs, paralogs, mutants, etc.) of SEQ ID NO: 12. Preferred fragments of (b) comprise an epitope from SEQ ID NO: 12. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more; preferably at least 16 to remove the signal peptide) from the N-terminus of SEQ ID NO: 12. Other fragments omit one or more domains of the protein (e.g. omission of a signal peptide as described above, of a cytoplasmic domain, of a transmembrane domain, or of an extracellular domain). In relation to SEQ ID NO: 12, distinct domains are residues: 1-16; 17-497; 128-289; 290-381; 394-485; and 394-497.

(13) MurG peptidoglycan transferase protein (CT761) One example of a ‘MurG’ protein is disclosed as SEQ ID NOs: 217 & 218 in WO 03/049762 (GenBank accession number: AAC68356, GI:3329223; ‘CT761’; SEQ ID NO: 13 in attached sequence listing). It is a UDP -N- acetylglucosamine -N- acetylmuramyl (pentapeptide) pyrophosphoryl undecaprenol -N- acetylglucosamine transferase. Preferred MurG proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) which is a fragment of at least n consecutive amino acids of SEQ ID NO: 13, wherein n is 7 or more (e.g. 8, 10, 12, 14, 16, 18, 20, 25, 30, 35, 40, 50, 60, 70, 80, 90, 100, 150, 200, 250 or more). These MurG proteins include variants (e.g. allelic variants, homologs, orthologs, paralogs, mutants, etc.) of SEQ ID NO: 13. Preferred fragments of (b) comprise an epitope from SEQ ID NO: 13. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 13. Other fragments omit one or more domains of the protein (e.g. omission of a signal peptide as described above, of a cytoplasmic domain, of a transmembrane domain, or of an extracellular domain). The MurG may be lipidated e.g. with undecaprenyl.

The immunogenicity of other known Chlamydia trachomatis antigens in component A may be improved by combination with two or more Chlamydia trachomatis antigens from either the first antigen group or the second antigen group. Such other known Chlamydia trachomatis antigens include a third antigen group consisting of (1) PGP3, (2) one or more PMP, (3) MOMP (CT681), (4) Cap1 (CT529); (5) GroEL-like hsp60 protein (Omp2); and (6) 60 kDa Cysteine rich protein (omcB). These antigens are referred to herein as the “third antigen group”.

The invention thus includes component A of a composition comprising a combination of Chlamydia trachomatis antigens, said combination selected from the group consisting of two, three, four, or five Chlamydia trachomatis antigens of the first antigen group and one, two, three, four, five or six Chlamydia trachomatis antigens of the third antigen group. Preferably, component A of the combination is selected from the group consisting of three, four, or five Chlamydia trachomatis antigens from the first antigen group and three, four, or five Chlamydia trachomatis antigens from the third antigen group. Still more preferably, component A of the combination consists of five Chlamydia trachomatis antigens from the first antigen group and three, four or five Chlamydia trachomatis antigens from the third antigen group.

The invention further includes component A of a composition comprising a combination of Chlamydia trachomatis antigens, said component A of the combination selected from the group consisting of two, three, four, five, six, seven, eight, nine, ten, eleven, twelve or thirteen Chlamydia trachomatis antigens of the second antigen group and one, two, three, four, five or six Chlamydia trachomatis antigens of the third antigen group. Preferably, component A of the combination is selected from the group consisting of three, four, or five Chlamydia trachomatis antigens from the second antigen group and three, four or five Chlamydia trachomatis from the third antigen group. Still more preferably, component A of the combination consists of five Chlamydia trachomatis antigens from the second antigen group and three, four or five Chlamydia trachomatis antigens of the third antigen group.

In either of the above combinations, preferably the Chlamydia trachomatis antigens from the third antigen group include Cap 1 (CT529). Or, alternatively, in either of the above combinations, preferably the Chlamydia trachomatis antigens from the third antigen group include MOMP (CT681). Each of the Chlamydia trachomatis antigens of the third antigen group are described in more detail below.

(1) Plasmid Encoded Protein (PGP3) One example of PGP3 sequence is disclosed in, for example, at Genbank entry GI 121541. Immunization with pgp3 is discussed in Ghaem-Maghami et al., (2003) Clin. Exp. Immunol. 132: 436-442 and Donati et al., (2003) Vaccine 21:1089-1093. One example of a PGP3 protein is set forth in attached sequence listing as SEQ ID NO: 14. Preferred PGP3 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) which is a fragment of at least n consecutive amino acids of SEQ ID NO: 14, wherein n is 7 or more (e.g. 8, 10, 12, 14, 16, 18, 20, 25, 30, 35, 40, 50, 60, 70, 80, 90, 100, 150, 200, 250 or more). These PGP3 proteins include variants (e.g. allelic variants, homologs, orthologs, paralogs, mutants, etc.) of SEQ ID NO: 14. Preferred fragments of (b) comprise an epitope from SEQ ID NO: 14. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 14. Other fragments omit one or more domains of the protein (e.g. omission of a signal peptide, of a cytoplasmic domain, of a transmembrane domain, or of an extracellular domain).

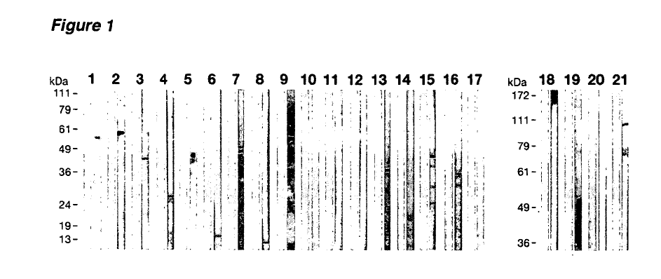

(2) Polymorphic Membrane Proteins (PMP) A family of nine Chlamydia trachomatis genes encoding predicted polymorphic membrane proteins (PMP) have been identified (pmpA to pmpI). See Stephens et al., Science (1998) 282:754-759, specifically FIG. 1. Examples of Amino acid sequences of the PMP genes are set forth as SEQ ID NOS: 15-23. (These sequences can also be found at Genbank Ref. Nos. GI 15605137 (pmpA), 15605138 (pmpB), 15605139 (pmpC), 15605546 (pmpD), 15605605 (pmpE), 15605606 (pmpF), 15605607 (pmpG), 15605608 (pmpH), and 15605610 (pmpH)). These PMP genes encode relatively large proteins (90 to 187 kDa in mass). The majority of these PMP proteins are predicted to be outer membrane proteins, and are thus also referred to as Predicted Outer Membrane Proteins. As used herein, PMP refers to one or more of the Chlamydia trachomatis pmp proteins (pmpA to pmpI) or an immunogenic fragment thereof. Preferably, the PMP protein used in the invention is pmpE or pmpI. Preferably, the PMP protein used in the invention comprises one or more of the fragments of pmpE or pmpI identified in International Patent Application PCT/US01/30345 (WO 02/28998) in Table 1 on page 20 (preferred fragments of pmpE) or Table 2 on page 21 (preferred fragments of pmpI).

Preferred PMP proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to one of the polypeptide sequences set forth as SEQ ID NOS: 15-23; and/or (b) which is a fragment of at least n consecutive amino acids of one of the polypeptide sequences set forth as SEQ ID NOS: 15-23, wherein n is 7 or more (e.g. 8, 10, 12, 14, 16, 18, 20, 25, 30, 35, 40, 50, 60, 70, 80, 90, 100, 150, 200, 250 or more). These PMP proteins include variants (e.g. allelic variants, homologs, orthologs, paralogs, mutants, etc.) of the polypeptide sequences set forth as SEQ ID NOS: 15-23. Preferred fragments of (b) comprise an epitope from one of the polypeptide sequences set forth as SEQ ID NOS: 15-23. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of one of the polypeptide sequences set forth as SEQ ID NOS: 15-23. Other fragments omit one or more domains of the protein (e.g. omission of a signal peptide, of a cytoplasmic domain, of a transmembrane domain, or of an extracellular domain).

(3) Major Outer Membrane Protein (MOMP) (CT681) One example of a MOMP sequence is disclosed as SEQ ID NOS 155 and 156 in International Patent Application No. PCT/IB02/05761 (WO 03/049762). The polypeptide sequence encoding MOMP is set forth in attached sequence listing as SEQ ID NO: 24. This protein is thought to function in vivo as a porin (See Bavoil et al, (1984) Infection and Immunity 44:479-485), and to be present during the whole life cycle of the bacteria (See Hatch et al., (1986) J. Bacteriol. 165:379-385). MOMP displays four variable domains (VD) surrounded by five constant regions that are highly conserved among serovars (See Stephens et al., (1987) J. Bacteriol. 169:3879-3885 and Yuan et al. (1989) Infection and Immunity 57: 1040-1049). In vitro and in vivo neutralizing B-cell epitopes have been mapped on VDs (See Baehr et al., (1988) PNAS USA 85:4000-4004; Lucero et al., (1985) Infection and Immunity 50:595-597; Zhang et al., (1987) J. Immunol. 138:575-581, Peterson et al., (1988) Infection and Immunity 56:885-891, Zhang et al., (1989) Infection and Immunity 57:636-638). T-cell epitopes have been identified in both variable and constant domains (See Allen et al., (1991) J. Immunol. 147:674-679 and Su et al., (1990) J. Exp. Med. 172:203-212).

Preferred MOMP proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 24; and/or (b) which is a fragment of at least n consecutive amino acids of SEQ ID NO: 24, wherein n is 7 or more (e.g. 8, 10, 12, 14, 16, 18, 20, 25, 30, 35, 40, 50, 60, 70, 80, 90, 100, 150, 200, 250 or more). These MOMP proteins include variants (e.g. allelic variants, homologs, orthologs, paralogs, mutants, etc.) of SEQ ID NO: 24. Preferred fragments of (b) comprise an epitope from SEQ ID NO: 24, preferably one or more of the B cell or T cell epitopes identified above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 24. Other fragments omit one or more domains of the protein (e.g. omission of a signal peptide, of a cytoplasmic domain, of a transmembrane domain, or of an extracellular domain). Other preferred fragments include one or more of the conserved constant regions identified above.

(4) Cap1 (CT529) The Chlamydia trachomatis Cap1 protein corresponds with the hypothetical open reading frame CT 529 and refers to Class I Accessible Protein-1. See Fling et al., (2001) PNAS 98(3): 1160-1165. Cap1 (CT529) is considered to be a Chlamydia effector protein present in the inclusion membrane. CT529 has also been shown to contain an epitope which in mouse vaccine experiments provides some protection against infection (see for example WO 02/082091). WO 02/48185 teaches that CT529 is a Type III Secretion System secreted protein. One example of a Cap1 protein is set forth herein as SEQ ID NO: 28. Predicted T-cell epitopes of Cap1 are identified in this reference as SEQ ID NO: 25 CSFIGGITYL, preferably SEQ ID NO: 26 SFIGGITYL, and SEQ ID NO: 27 SIIGGITYL.

Preferred Cap1 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 28; and/or (b) which is a fragment of at least n consecutive amino acids of SEQ ID NO: 28, wherein n is 7 or more (e.g. 8, 10, 12, 14, 16, 18, 20, 25, 30, 35, 40, 50, 60, 70, 80, 90, 100, 150, 200, 250 or more). These Cap1 proteins include variants (e.g. allelic variants, homologs, orthologs, paralogs, mutants, etc.) of SEQ ID NO: 28. Preferred fragments of (b) comprise an epitope from SEQ ID NO: 28. Preferred T-cell epitopes include one or more of the T-cell epitopes identified above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 28. Other fragments omit one or more domains of the protein (e.g. omission of a signal peptide, of a cytoplasmic domain, of a transmembrane domain, or of an extracellular domain).

(5) GroEL-like hsp60 protein One example of a Chlamydia trachomatis GroEL-like hsp60 protein is set forth herein as SEQ ID NO: 29. The role of Hsp60 in chlamydial infection is further described in, for example, Hessel, et al., (2001) Infection and Immunity 69(8): 4996-5000; Eckert, et al., (1997) J. Infectious Disease 175:1453-1458, Domeika et al., (1998) J. of Infectious Diseases 177:714-719; Deane et al., (1997) Clin. Exp. Immunol. 109(3): 439-445, and Peeling et al., (1997) J. Infect. Dis. 175(5):1153-1158. Immunization of guinea pig models with recombinant Hsp60 is described in Rank et al., (1995) Incest Ophthalmol. Vis. Sci. 36(7):1344-1351. B-cell epitopes of Hsp60 are identified in Yi et al., (1993) Infection & Immunity 61(3):1117-1120.

Preferred hsp60 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 29; and/or (b) which is a fragment of at least n consecutive amino acids of SEQ ID NO: 29, wherein n is 7 or more (e.g. 8, 10, 12, 14, 16, 18, 20, 25, 30, 35, 40, 50, 60, 70, 80, 90, 100, 150, 200, 250 or more). These hsp60 proteins include variants (e.g. allelic variants, homologs, orthologs, paralogs, mutants, etc.) of SEQ ID NO: 29. Preferred fragments of (b) comprise an epitope from SEQ ID NO: 29, including one or more of the epitopes identified in the references discussed above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 29. Other fragments omit one or more domains of the protein (e.g. omission of a signal peptide, of a cytoplasmic domain, of a transmembrane domain, or of an extracellular domain). Other preferred fragments comprise a polypeptide sequence which does not cross-react with related human proteins.

(6) 60 kDa Cysteine rich protein (OmcB) (CT443) One example of a Chlamydia trachomatis 60 kDa Cysteine rich protein is set forth herein as SEQ ID NO: 30. This protein is also generally referred to as OmcB, Omp2 or CT 443. The role of OmcB in chlamydial infection is further described in, for example, Stephens et al., (2001) Molecular Microbiology 40(3):691-699; Millman, et al., (2001) J. of Bacteriology 183(20):5997-6008; Mygind, et al., Journal of Bacteriology (1998) 180(21):5784-5787; Bas, et al., Journal of Clinical Microbiology (2001) 39(11):4082-4085 and Goodall, et al., Clin. Exp. Immunol. (2001) 126:488-493.

Preferred OmcB proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 30; and/or (b) which is a fragment of at least n consecutive amino acids of SEQ ID NO: 30, wherein n is 7 or more (e.g. 8, 10, 12, 14, 16, 18, 20, 25, 30, 35, 40, 50, 60, 70, 80, 90, 100, 150, 200, 250 or more). These OmcB proteins include variants (e.g. allelic variants, homologs, orthologs, paralogs, mutants, etc.) of SEQ ID NO: 30. Preferred fragments of (b) comprise an epitope from SEQ ID NO: 30, including one or more of the epitopes identified in the references discussed above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 30. Other fragments omit one or more domains of the protein (e.g. omission of a signal peptide, of a cytoplasmic domain, of a transmembrane domain, or of an extracellular domain).

The immunogenicity of other Chlamydia trachomatis antigens of known and unknown biological function may be improved within component A of the combination of Chlamydia trachomatis antigens by combination with two or more Chlamydia trachomatis antigens from either the first antigen group and/or the second and/or the third antigen group. Such other Chlamydia trachomatis antigens of known and unknown biological function include a fourth antigen group consisting of (1) CT559 (YscJ); (2) CT600 (Pal); (3) CT541 (Mip); (4) CT623 (CHLPN 76kDA homologue) (5) CT700 (Hypothetical protein). (6) CT266 (Hypothetical protein); (7) CT077 (Hypothetical protein); (8) CT713 (PorB); and (9) CT165 (Hypothetical protein). These antigens are referred to as the “fourth antigen group”.

YscJ (CT559) One example of ‘YscJ’ protein is disclosed as SEQ ID NOs: 199 & 200 in WO 03/049762 (GenBank accession number: AAC68161.1 GI:3329000; ‘CT559’; SEQ ID NO: 31 in attached sequence listing). Preferred YscJ proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 31; and/or (b) which is a fragment of at least n consecutive amino acids of SEQ ID NO: 31, wherein n is 7 or more (e.g. 8, 10, 12, 14, 16, 18, 20, 25, 30, 35, 40, 50, 60, 70, 80, 90, 100, 150, 200, 250 or more). These YscJ proteins include variants (e.g. allelic variants, homologs, orthologs, paralogs, mutants, etc.) of SEQ ID NO: 31. Preferred fragments of (b) comprise an epitope from SEQ ID NO: 31. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 31. Other fragments omit one or more domains of the protein (e.g. omission of a signal peptide, of a cytoplasmic domain, of a transmembrane domain, or of an extracellular domain).

Pal (CT600) One example of a ‘Pal’ protein is disclosed as SEQ ID NOs: 173 & 174 in WO 03/049762 (GenBank accession number: AAC68202.1 GI:3329044 ‘CT600’; SEQ ID NO: 32 in attached sequence listing). Preferred Pal proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 32; and/or (b) which is a fragment of at least n consecutive amino acids of SEQ ID NO: 32, wherein n is 7 or more (e.g. 8, 10, 12, 14, 16, 18, 20, 25, 30, 35, 40, 50, 60, 70, 80, 90, 100, 150, 200, 250 or more). These Pal proteins include variants (e.g. allelic variants, homologs, orthologs, paralogs, mutants, etc.) of SEQ ID NO: 32. Preferred fragments of (b) comprise an epitope from SEQ ID NO: 32. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 32. Other fragments omit one or more domains of the protein (e.g. omission of a signal peptide, of a cytoplasmic domain, of a transmembrane domain, or of an extracellular domain).

Mip (CT541) One example of a ‘Mip’ protein is disclosed as SEQ ID NOs: 149 & 150 in WO 03/049762 (GenBank accession number: AAC68143.1 GI:3328979 ‘CT541’; SEQ ID NO: 33 in attached sequence listing). Preferred Mip proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 33; and/or (b) which is a fragment of at least n consecutive amino acids of SEQ ID NO: 33, wherein n is 7 or more (e.g. 8, 10, 12, 14, 16, 18, 20, 25, 30, 35, 40, 50, 60, 70, 80, 90, 100, 150, 200, 250 or more). These Mip proteins include variants (e.g. allelic variants, homologs, orthologs, paralogs, mutants, etc.) of SEQ ID NO: 33. Preferred fragments of (b) comprise an epitope from SEQ ID NO: 33. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 33. Other fragments omit one or more domains of the protein (e.g. omission of a signal peptide, of a cytoplasmic domain, of a transmembrane domain, or of an extracellular domain).

CHLPN (76 kDa) (CT623) One example of a CHLPN (76 kDa protein) is disclosed as SEQ ID NOs: 163 & 164 in WO 03/049762 (GenBank accession number: AAC68227.2 GI:6578109 ‘CT623’; SEQ ID NO: 34 in the attached sequence listing). Preferred CHLPN (76 kDa protein proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 34; and/or (b) which is a fragment of at least n consecutive amino acids of SEQ ID NO: 34, wherein n is 7 or more (e.g. 8, 10, 12, 14, 16, 18, 20, 25, 30, 35, 40, 50, 60, 70, 80, 90, 100, 150, 200, 250 or more). These CHLPN (76 kDa protein) proteins include variants (e.g. allelic variants, homologs, orthologs, paralogs, mutants, etc.) of SEQ ID NO: 34. Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34. Other fragments omit one or more domains of the protein (e.g. omission of a signal peptide, of a cytoplasmic domain, of a transmembrane domain, or of an extracellular domain).

Hypothetical Protein (CT700) One example of a CT700 Hypothetical Protein is disclosed as SEQ ID NOs 261 & 262 in WO 03/049762 (GenBank accession number: AAC68295.1 GI:3329154 ‘CT700’; SEQ ID NO: 35 in attached sequence listing). Preferred CT700 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 35; and/or (b) which is a fragment of at least n consecutive amino acids of SEQ ID NO: 35, wherein n is 7 or more (e.g. 8, 10, 12, 14, 16, 18, 20, 25, 30, 35, 40, 50, 60, 70, 80, 90, 100, 150, 200, 250 or more). These CT700 Hypothetical proteins include variants (e.g. allelic variants, homologs, orthologs, paralogs, mutants, etc.) of SEQ ID NO: 35. Preferred fragments of (b) comprise an epitope from SEQ ID NO: 35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 35. Other fragments omit one or more domains of the protein (e.g. omission of a signal peptide, of a cytoplasmic domain, of a transmembrane domain, or of an extracellular domain).

Hypothetical Protein (CT 266) One example of a CT266 Hypothetical Protein is disclosed as SEQ ID NOs 77 & 78 in WO 03/049762 (GenBank accession number: AAC67859.1 GI:3328678 ‘CT266’; SEQ ID NO: 36 in attached sequence listing). Preferred CT266 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 36; and/or (b) which is a fragment of at least n consecutive amino acids of SEQ ID NO: 36, wherein n is 7 or more (e.g. 8, 10, 12, 14, 16, 18, 20, 25, 30, 35, 40, 50, 60, 70, 80, 90, 100, 150, 200, 250 or more). These CT266 Hypothetical proteins include variants (e.g. allelic variants, homologs, orthologs, paralogs, mutants, etc.) of SEQ ID NO: 36. Preferred fragments of (b) comprise an epitope from SEQ ID NO: 36. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 36. Other fragments omit one or more domains of the protein (e.g. omission of a signal peptide, of a cytoplasmic domain, of a transmembrane domain, or of an extracellular domain).

Hypothetical Protein (CT077) One example of a CT077 Hypothetical Protein is disclosed as SEQ ID NOs 65 & 66 in WO 03/049762 (GenBank accession number: AAC67668.1 GI:3328472 ‘CT077’; SEQ ID NO: 37 in attached sequence listing). Preferred CT077 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37; and/or (b) which is a fragment of at least n consecutive amino acids of SEQ ID NO: 37, wherein n is 7 or more (e.g. 8, 10, 12, 14, 16, 18, 20, 25, 30, 35, 40, 50, 60, 70, 80, 90, 100, 150, 200, 250 or more). These CT077 Hypothetical proteins include variants (e.g. allelic variants, homologs, orthologs, paralogs, mutants, etc.) of SEQ ID NO: 37. Preferred fragments of (b) comprise an epitope from SEQ ID NO: 37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 37. Other fragments omit one or more domains of the protein (e.g. omission of a signal peptide, of a cytoplasmic domain, of a transmembrane domain, or of an extracellular domain).

PorB (CT713) One example of a PorB Protein is disclosed as SEQ ID NOs 201 & 202 in WO 03/049762 (GenBank accession number: AAC68308.1 GI:3329169 ‘CT713’; SEQ ID NO: 38 in attached sequence listing). Preferred PorB proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40; and/or (b) which is a fragment of at least n consecutive amino acids of SEQ ID NO: 40, wherein n is 7 or more (e.g. 8, 10, 12, 14, 16, 18, 20, 25, 30, 35, 40, 50, 60, 70, 80, 90, 100, 150, 200, 250 or more). These PorB proteins include variants (e.g. allelic variants, homologs, orthologs, paralogs, mutants, etc.) of SEQ ID NO: 40. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 40. Other fragments omit one or more domains of the protein (e.g. omission of a signal peptide, of a cytoplasmic domain, of a transmembrane domain, or of an extracellular domain). The PorB protein is highly conserved among Chlamydia trachomatis serovars. It is localized in the Chlamydial outer membrane surface and is a target of neutralizing antibody responses in vitro which is a correlate of immune-mediated protection. Antibody to the neutralizing antigenic determinants show cross-reactivity to Chlamydia pneumoniae PorB supporting its structural conservation across the species (see Kawa et al (2004) Vaccine 22; 4282-4286).

Hypothetical Protein (CT165) One example of a CT165 Hypothetical Protein is disclosed (GenBank accession number: AAC67756.1 GI:3328568 CT165′; SEQ ID NO: 39 in attached sequence listing). Preferred Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 39; and/or (b) which is a fragment of at least n consecutive amino acids of SEQ ID NO: 39, wherein n is 7 or more (e.g. 8, 10, 12, 14, 16, 18, 20, 25, 30, 35, 40, 50, 60, 70, 80, 90, 100, 150, 200, 250 or more). These CT165 Hypothetical proteins include variants (e.g. allelic variants, homologs, orthologs, paralogs, mutants, etc.) of SEQ ID NO: 39. Preferred fragments of (b) comprise an epitope from SEQ ID NO: 39. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 39. Other fragments omit one or more domains of the protein (e.g. omission of a signal peptide, of a cytoplasmic domain, of a transmembrane domain, or of an extracellular domain).

The immunogenicity of other Chlamydia trachomatis antigens of known and unknown biological function may be improved within component A of the combination of Chlamydia trachomatis antigens by combination with two or more Chlamydia trachomatis antigens from either the first antigen group and/or the second and/or the third antigen group and/or the fourth antigen group. Such other Chlamydia trachomatis antigens of known and unknown biological function include a fifth antigen group consisting of: (1) CT082 (hypothetical); (2) CT181 (Hypothetical); (3) CT050 (Hypothetical); (4) CT157 (Phospholipase D superfamily); and (5) CT128 (AdK adenylate cyclase).

Hypothetical Protein (CT082) One example of a CT082 Hypothetical Protein is disclosed as (GenBank accession number: AAC67673.1 GI:3328477 ‘CT082’; SEQ ID NO: 40 in attached sequence listing). Preferred CT082 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40; and/or (b) which is a fragment of at least n consecutive amino acids of SEQ ID NO: 41, wherein n is 7 or more (e.g. 8, 10, 12, 14, 16, 18, 20, 25, 30, 35, 40, 50, 60, 70, 80, 90, 100, 150, 200, 250 or more). These CT082 Hypothetical proteins include variants (e.g. allelic variants, homologs, orthologs, paralogs, mutants, etc.) of SEQ ID NO: 40. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 40. Other fragments omit one or more domains of the protein (e.g. omission of a signal peptide, of a cytoplasmic domain, of a transmembrane domain, or of an extracellular domain).

Hypothetical Protein (CT181) One example of a CT181 Hypothetical Protein is disclosed as SEQ ID NOs 245 & 246 in WO 03/049762 (GenBank accession number: AAC67772.1 GI:3328585 ‘CT181’; SEQ ID NO: 41 in attached sequence listing). Preferred CT181 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 41; and/or (b) which is a fragment of at least n consecutive amino acids of SEQ ID NO: 41, wherein n is 7 or more (e.g. 8, 10, 12, 14, 16, 18, 20, 25, 30, 35, 40, 50, 60, 70, 80, 90, 100, 150, 200, 250 or more). These CT181 Hypothetical proteins include variants (e.g. allelic variants, homologs, orthologs, paralogs, mutants, etc.) of SEQ ID NO: 41. Preferred fragments of (b) comprise an epitope from SEQ ID NO: 41. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 41. Other fragments omit one or more domains of the protein (e.g. omission of a signal peptide, of a cytoplasmic domain, of a transmembrane domain, or of an extracellular domain).

Hypothetical Protein (CT050) One example of a CT050 Hypothetical Protein is disclosed as (GenBank accession number: AAC67641.1 GI:3328442 ‘CT050’; SEQ ID NO: 42 in attached sequence listing). Preferred CT050 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 42; and/or (b) which is a fragment of at least n consecutive amino acids of SEQ ID NO: 42, wherein n is 7 or more (e.g. 8, 10, 12, 14, 16, 18, 20, 25, 30, 35, 40, 50, 60, 70, 80, 90, 100, 150, 200, 250 or more). These CT050 Hypothetical proteins include variants (e.g. allelic variants, homologs, orthologs, paralogs, mutants, etc.) of SEQ ID NO: 42. Preferred fragments of (b) comprise an epitope from SEQ ID NO: 42. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 43. Other fragments omit one or more domains of the protein (e.g. omission of a signal peptide, of a cytoplasmic domain, of a transmembrane domain, or of an extracellular domain).

Phospholipase D SuperFamily (CT157) One example of a Phospholipase D SuperFamily Protein is disclosed as (GenBank accession number: AAC67748.1 GI:3328559 ‘CT157’; SEQ ID NO: 43 in attached sequence listing). Preferred Phospholipase D SuperFamily proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 43; and/or (b) which is a fragment of at least n consecutive amino acids of SEQ ID NO: 43, wherein n is 7 or more (e.g. 8, 10, 12, 14, 16, 18, 20, 25, 30, 35, 40, 50, 60, 70, 80, 90, 100, 150, 200, 250 or more). These Phospholipase D SuperFamily proteins include variants (e.g. allelic variants, homologs, orthologs, paralogs, mutants, etc.) of SEQ ID NO: 43. Preferred fragments of (b) comprise an epitope from SEQ ID NO: 44. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 43. Other fragments omit one or more domains of the protein (e.g. omission of a signal peptide, of a cytoplasmic domain, of a transmembrane domain, or of an extracellular domain).

Ad % (Adenylate Kinase) (CT128) One example of an Adenylate Kinase Protein is disclosed as (GenBank accession number: AAC67719.1 GI:3328527 ‘CT128’; SEQ ID NO: 44 in attached sequence listing). Preferred Adenylate Kinase proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 44; and/or (b) which is a fragment of at least n consecutive amino acids of SEQ ID NO: 44, wherein n is 7 or more (e.g. 8, 10, 12, 14, 16, 18, 20, 25, 30, 35, 40, 50, 60, 70, 80, 90, 100, 150, 200, 250 or more). These Adenylate Kinase proteins include variants (e.g. allelic variants, homologs, orthologs, paralogs, mutants, etc.) of SEQ ID NO: 44. Preferred fragments of (b) comprise an epitope from SEQ ID NO: 44. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 44. Other fragments omit one or more domains of the protein (e.g. omission of a signal peptide, of a cytoplasmic domain, of a transmembrane domain, or of an extracellular domain).

The immunogenicity of other Chlamydia trachomatis antigens of known and unknown biological function may be improved within component A of the combination of Chlamydia trachomatis antigens by combination with two or more Chlamydia trachomatis antigens from either the first antigen group and/or the second and/or the third antigen group and/or the fourth antigen group and/or the fifth antigen group. Such other Chlamydia trachomatis antigens of known and unknown biological function include a sixth antigen group consisting of (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE).

Hypothetical Protein (CT153) One example of a CT153 Hypothetical Protein is disclosed as (GenBank accession number: AAC67744.1 GI:3328555 ‘CT153’; SEQ ID NO: 45 in attached sequence listing). Preferred CT153 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 45; and/or (b) which is a fragment of at least n consecutive amino acids of SEQ ID NO: 45, wherein n is 7 or more (e.g. 8, 10, 12, 14, 16, 18, 20, 25, 30, 35, 40, 50, 60, 70, 80, 90, 100, 150, 200, 250 or more). These CT153 Hypothetical proteins include variants (e.g. allelic variants, homologs, orthologs, paralogs, mutants, etc.) of SEQ ID NO: 45. Preferred fragments of (b) comprise an epitope from SEQ ID NO: 45. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 45. Other fragments omit one or more domains of the protein (e.g. omission of a signal peptide, of a cytoplasmic domain, of a transmembrane domain, or of an extracellular domain).

Hypothetical Protein (CT262) One example of a CT262 Hypothetical Protein is disclosed as (GenBank accession number: AAC67835.1 GI:3328652′CT262′; SEQ ID NO: 46 in attached sequence listing). Preferred CT262 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 47; and/or (b) which is a fragment of at least n consecutive amino acids of SEQ ID NO: 46, wherein n is 7 or more (e.g. 8, 10, 12, 14, 16, 18, 20, 25, 30, 35, 40, 50, 60, 70, 80, 90, 100, 150, 200, 250 or more). These CT262 Hypothetical proteins include variants (e.g. allelic variants, homologs, orthologs, paralogs, mutants, etc.) of SEQ ID NO: 46. Preferred fragments of (b) comprise an epitope from SEQ ID NO: 46. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 46. Other fragments omit one or more domains of the protein (e.g. omission of a signal peptide, of a cytoplasmic domain, of a transmembrane domain, or of an extracellular domain).

Hypothetical Protein (CT276) One example of a CT276 Hypothetical Protein is disclosed as (GenBank accession number: AAC67869.1 GI:3328689 ‘CT276’; SEQ ID NO: 47 in attached sequence listing). Preferred CT276 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 47; and/or (b) which is a fragment of at least n consecutive amino acids of SEQ ID NO: 47, wherein n is 7 or more (e.g. 8, 10, 12, 14, 16, 18, 20, 25, 30, 35, 40, 50, 60, 70, 80, 90, 100, 150, 200, 250 or more). These CT276 Hypothetical proteins include variants (e.g. allelic variants, homologs, orthologs, paralogs, mutants, etc.) of SEQ ID NO: 47. Preferred fragments of (b) comprise an epitope from SEQ ID NO: 47. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 48. Other fragments omit one or more domains of the protein (e.g. omission of a signal peptide, of a cytoplasmic domain, of a transmembrane domain, or of an extracellular domain).

Hypothetical Protein (CT296) One example of a CT296 Hypothetical Protein is disclosed as (GenBank accession number: AAC67889.1 GI:3328711 ‘CT296’; SEQ ID NO: 48 in attached sequence listing). Preferred CT296 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 48; and/or (b) which is a fragment of at least n consecutive amino acids of SEQ ID NO: 48, wherein n is 7 or more (e.g. 8, 10, 12, 14, 16, 18, 20, 25, 30, 35, 40, 50, 60, 70, 80, 90, 100, 150, 200, 250 or more). These CT296 Hypothetical proteins include variants (e.g. allelic variants, homologs, orthologs, paralogs, mutants, etc.) of SEQ ID NO: 48. Preferred fragments of (b) comprise an epitope from SEQ ID NO: 48. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 48. Other fragments omit one or more domains of the protein (e.g. omission of a signal peptide, of a cytoplasmic domain, of a transmembrane domain, or of an extracellular domain).

Hypothetical Protein (CT372) One example of a CT372 Hypothetical Protein is disclosed as SEQ ID NOs 187 & 188 in WO 03/049762 (GenBank accession number: AAC67968.1 GI:3328796 ‘CT372’; SEQ ID NO: 49 in attached sequence listing). Preferred CT372 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 50; and/or (b) which is a fragment of at least n consecutive amino acids of SEQ ID NO: 49, wherein n is 7 or more (e.g. 8, 10, 12, 14, 16, 18, 20, 25, 30, 35, 40, 50, 60, 70, 80, 90, 100, 150, 200, 250 or more). These CT372 Hypothetical proteins include variants (e.g. allelic variants, homologs, orthologs, paralogs, mutants, etc.) of SEQ ID NO: 49. Preferred fragments of (b) comprise an epitope from SEQ ID NO: 49. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 49. Other fragments omit one or more domains of the protein (e.g. omission of a signal peptide, of a cytoplasmic domain, of a transmembrane domain, or of an extracellular domain).

Putative Outer Membrane Protein A (PmpA) (CT412) One example of a PmpA Protein is disclosed as SEQ ID NOs 89 & 90 in WO 03/049762 (GenBank accession number: AAC68009.1 GI:3328840 ‘CT412’; SEQ ID NO: 50 in attached sequence listing and also SEQ ID No 15 above). Preferred PmpA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 50; and/or (b) which is a fragment of at least n consecutive amino acids of SEQ ID NO: 50, wherein n is 7 or more (e.g. 8, 10, 12, 14, 16, 18, 20, 25, 30, 35, 40, 50, 60, 70, 80, 90, 100, 150, 200, 250 or more). These PmpA proteins include variants (e.g. allelic variants, homologs, orthologs, paralogs, mutants, etc.) of SEQ ID NO: 50. Preferred fragments of (b) comprise an epitope from SEQ ID NO: 50. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 50. Other fragments omit one or more domains of the protein (e.g. omission of a signal peptide, of a cytoplasmic domain, of a transmembrane domain, or of an extracellular domain).

Oligopeptide Binding Lipoprotein (CT480) One example of an OligoPeptide Binding Protein is disclosed as SEQ ID NOs 141 & 142 in WO 03/049762 (GenBank accession number: AAC68080.1 GI:3328915 ‘CT480’; SEQ ID NO: 51 in attached sequence listing). Preferred OligoPeptide Binding proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 51; and/or (b) which is a fragment of at least n consecutive amino acids of SEQ ID NO: 51, wherein n is 7 or more (e.g. 8, 10, 12, 14, 16, 18, 20, 25, 30, 35, 40, 50, 60, 70, 80, 90, 100, 150, 200, 250 or more). These OligoPeptide Binding proteins include variants (e.g. allelic variants, homologs, orthologs, paralogs, mutants, etc.) of SEQ ID NO: 51. Preferred fragments of (b) comprise an epitope from SEQ ID NO: 51. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 51. Other fragments omit one or more domains of the protein (e.g. omission of a signal peptide, of a cytoplasmic domain, of a transmembrane domain, or of an extracellular domain).

Hypothetical Protein (CT548) One example of a Hypothetical Protein is disclosed as SEQ ID NOs 153 & 154 in WO 03/049762 (GenBank accession number: AAC68150.1 GI:3328987 ‘CT548’; SEQ ID NO: 52 in attached sequence listing). Preferred CT548 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 52; and/or (b) which is a fragment of at least n consecutive amino acids of SEQ ID NO: 52, wherein n is 7 or more (e.g. 8, 10, 12, 14, 16, 18, 20, 25, 30, 35, 40, 50, 60, 70, 80, 90, 100, 150, 200, 250 or more). These CT548 Hypothetical proteins include variants (e.g. allelic variants, homologs, orthologs, paralogs, mutants, etc.) of SEQ ID NO: 52. Preferred fragments of (b) comprise an epitope from SEQ ID NO: 52. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 52. Other fragments omit one or more domains of the protein (e.g. omission of a signal peptide, of a cytoplasmic domain, of a transmembrane domain, or of an extracellular domain).

Hypothetical Protein (CT043) One example of a CT043 Hypothetical Protein is disclosed as (GenBank accession number: AAC67634.1 GI:3328435 ‘CT043’; SEQ ID NO: 53 in attached sequence listing). Preferred CT043 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 53; and/or (b) which is a fragment of at least n consecutive amino acids of SEQ ID NO: 53, wherein n is 7 or more (e.g. 8, 10, 12, 14, 16, 18, 20, 25, 30, 35, 40, 50, 60, 70, 80, 90, 100, 150, 200, 250 or more). These CT043 Hypothetical proteins include variants (e.g. allelic variants, homologs, orthologs, paralogs, mutants, etc.) of SEQ ID NO: 53. Preferred fragments of (b) comprise an epitope from SEQ ID NO: 53. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 53. Other fragments omit one or more domains of the protein (e.g. omission of a signal peptide, of a cytoplasmic domain, of a transmembrane domain, or of an extracellular domain).

Hypothetical Protein (CT635) One example of a CT635 Hypothetical Protein is disclosed as (GenBank accession number: AAC68239.1 GI:3329083 ‘CT635’; SEQ ID NO: 54 in attached sequence listing). Preferred CT635 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 54; and/or (b) which is a fragment of at least n consecutive amino acids of SEQ ID NO: 54, wherein n is 7 or more (e.g. 8, 10, 12, 14, 16, 18, 20, 25, 30, 35, 40, 50, 60, 70, 80, 90, 100, 150, 200, 250 or more). These CT635 Hypothetical proteins include variants (e.g. allelic variants, homologs, orthologs, paralogs, mutants, etc.) of SEQ ID NO: 54. Preferred fragments of (b) comprise an epitope from SEQ ID NO: 54. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 54. Other fragments omit one or more domains of the protein (e.g. omission of a signal peptide, of a cytoplasmic domain, of a transmembrane domain, or of an extracellular domain).

Metalloprotease (CT859) One example of a Metalloprotease Protein is disclosed as (GenBank accession number: ‘CT859’ AAC68457.1 GI:3329333; SEQ ID NO: 55 in attached sequence listing). Preferred Metalloprotease proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 55; and/or (b) which is a fragment of at least n consecutive amino acids of SEQ ID NO: 55, wherein n is 7 or more (e.g. 8, 10, 12, 14, 16, 18, 20, 25, 30, 35, 40, 50, 60, 70, 80, 90, 100, 150, 200, 250 or more). These Metalloprotease proteins include variants (e.g. allelic variants, homologs, orthologs, paralogs, mutants, etc.) of SEQ ID NO: 55. Preferred fragments of (b) comprise an epitope from SEQ ID NO: 55. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 55. Other fragments omit one or more domains of the protein (e.g. omission of a signal peptide, of a cytoplasmic domain, of a transmembrane domain, or of an extracellular domain).

Hypothetical Protein (CT671) One example of a CT671 Hypothetical Protein is disclosed as (GenBank accession number: AAC68266.1 GI:3329122 ‘CT671’; SEQ ID NO: 56 in attached sequence listing). Preferred CT671 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 56; and/or (b) which is a fragment of at least n consecutive amino acids of SEQ ID NO: 56, wherein n is 7 or more (e.g. 8, 10, 12, 14, 16, 18, 20, 25, 30, 35, 40, 50, 60, 70, 80, 90, 100, 150, 200, 250 or more). These CT671 Hypothetical proteins include variants (e.g. allelic variants, homologs, orthologs, paralogs, mutants, etc.) of SEQ ID NO: 56. Preferred fragments of (b) comprise an epitope from SEQ ID NO: 56. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 56. Other fragments omit one or more domains of the protein (e.g. omission of a signal peptide, of a cytoplasmic domain, of a transmembrane domain, or of an extracellular domain). WO 02/48185 teaches that CT671 is a Type III Secretion System secreted protein.

Hypothetical Protein (CT016) One example of a CT016 Hypothetical Protein is disclosed as (GenBank accession number: AAC67606.1 GI:3328405 ‘CT016’; SEQ ID NO: 57 in attached sequence listing). Preferred CT016 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 57; and/or (b) which is a fragment of at least n consecutive amino acids of SEQ ID NO: 57, wherein n is 7 or more (e.g. 8, 10, 12, 14, 16, 18, 20, 25, 30, 35, 40, 50, 60, 70, 80, 90, 100, 150, 200, 250 or more). These CT016 Hypothetical proteins include variants (e.g. allelic variants, homologs, orthologs, paralogs, mutants, etc.) of SEQ ID NO: 57. Preferred fragments of (b) comprise an epitope from SEQ ID NO: 57. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 57. Other fragments omit one or more domains of the protein (e.g. omission of a signal peptide, of a cytoplasmic domain, of a transmembrane domain, or of an extracellular domain). WO 02/48185 teaches that CT016 is a Type III Secretion System secreted protein.

Hypothetical Protein (CT017) One example of a CT017 Hypothetical Protein is disclosed as (GenBank accession number: AAC67607.1 GI:3328406 ‘CT017’; SEQ ID NO: 58 in attached sequence listing). Preferred CT017 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 58; and/or (b) which is a fragment of at least n consecutive amino acids of SEQ ID NO: 58, wherein n is 7 or more (e.g. 8, 10, 12, 14, 16, 18, 20, 25, 30, 35, 40, 50, 60, 70, 80, 90, 100, 150, 200, 250 or more). These CT017 Hypothetical proteins include variants (e.g. allelic variants, homologs, orthologs, paralogs, mutants, etc.) of SEQ ID NO: 58. Preferred fragments of (b) comprise an epitope from SEQ ID NO: 58. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 58. Other fragments omit one or more domains of the protein (e.g. omission of a signal peptide, of a cytoplasmic domain, of a transmembrane domain, or of an extracellular domain).

Hypothetical Protein (CT043) One example of aCT043 Hypothetical Protein is disclosed as (GenBank accession number: AAC67634.1 GI:3328435 ‘CT043’; SEQ ID NO: 59 in attached sequence listing). Preferred CT043 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 59; and/or (b) which is a fragment of at least n consecutive amino acids of SEQ ID NO: 59, wherein n is 7 or more (e.g. 8, 10, 12, 14, 16, 18, 20, 25, 30, 35, 40, 50, 60, 70, 80, 90, 100, 150, 200, 250 or more). These CT043 Hypothetical proteins include variants (e.g. allelic variants, homologs, orthologs, paralogs, mutants, etc.) of SEQ ID NO: 59. Preferred fragments of (b) comprise an epitope from SEQ ID NO: 59. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 59. Other fragments omit one or more domains of the protein (e.g. omission of a signal peptide, of a cytoplasmic domain, of a transmembrane domain, or of an extracellular domain).

Hypothetical Protein (CT082) This hypothetical protein is already discussed above as SEQ ID No 39.

Hypothetical Protein (CT548) One example of a Hypothetical Protein is disclosed as (GenBank accession number: AAC68150.1 GI:3328987 ‘CT548’; SEQ ID NO: 60 in attached sequence listing). Preferred Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 60; and/or (b) which is a fragment of at least n consecutive amino acids of SEQ ID NO: 60, wherein n is 7 or more (e.g. 8, 10, 12, 14, 16, 18, 20, 25, 30, 35, 40, 50, 60, 70, 80, 90, 100, 150, 200, 250 or more). These Hypothetical proteins include variants (e.g. allelic variants, homologs, orthologs, paralogs, mutants, etc.) of SEQ ID NO: 60. Preferred fragments of (b) comprise an epitope from SEQ ID NO: 60. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 60. Other fragments omit one or more domains of the protein (e.g. omission of a signal peptide, of a cytoplasmic domain, of a transmembrane domain, or of an extracellular domain).

LcrE (CT089) This Low Calcium Response Element protein is discussed above as SEQ ID NO: 2 and SEQ ID NO 40.

PmpD (CT812) This polymorphic membrane protein D is discussed above as SEQ ID NO: 18 (CT812).

PmpE (CT869) This polymorphic membrane protein E is discussed above as SEQ ID NO: 19.

The invention includes a composition comprising component A of Chlamydia trachomatis antigens in a combination of Chlamydia trachomatis antigens, said component A of the combination selected from the group consisting of two, three, four, or five Chlamydia trachomatis antigens of the first antigen group and one, two, three, four, or five antigens of the fourth antigen group.

The invention includes a composition comprising component A of Chlamydia trachomatis antigens in a combination of Chlamydia trachomatis antigens, said component A of the combination selected from the group consisting of two, three, four, or five Chlamydia trachomatis antigens of the first antigen group and one, two, three, four or five antigens of the fifth antigen group.

The invention includes a composition comprising component A of Chlamydia trachomatis antigens in a combination of Chlamydia trachomatis antigens, said component A of the combination selected from the group consisting of two, three, four, or five Chlamydia trachomatis antigens of the first antigen group and one, two, three, four or five antigens of the sixth antigen group.

The invention includes a composition comprising component A of Chlamydia trachomatis antigens in a combination of Chlamydia trachomatis antigens, said component A of the combination selected from the group consisting of two, three, four, or five Chlamydia trachomatis antigens of the second antigen group and one, two, three, four or five antigens of the fourth antigen group.

The invention includes a composition comprising component A of Chlamydia trachomatis antigens in a combination of Chlamydia trachomatis antigens, said component A of the combination selected from the group consisting of two, three, four, or five Chlamydia trachomatis antigens of the second antigen group and one, two, three, four or five antigens of the fifth antigen group.

The invention includes a composition comprising component A of Chlamydia trachomatis antigens in a combination of Chlamydia trachomatis antigens, said component A of the combination selected from the group consisting of two, three, four, or five Chlamydia trachomatis antigens of the second antigen group and one, two, three, four or five antigens of the sixth antigen group.

The invention thus includes a composition comprising component A of Chlamydia trachomatis antigens in a combination of Chlamydia trachomatis antigens, said component A of the combination selected from the group consisting of two, three, four, or five Chlamydia trachomatis antigens of the first antigen group and one, two, three, four, five or six Chlamydia trachomatis antigens of the third antigen group and one, two, three, four, five, six, seven, eight, nine or ten antigens of the fourth antigen group and one, two, three, four or five Chlamydia trachomatis antigens of the fifth antigen group and one, two, three, four, five, six, seven, eight, nine, ten, eleven or twelve antigens of the sixth antigen group.

Preferably, component A of the combination is selected from the group consisting of three, four, or five Chlamydia trachomatis antigens from the first antigen group and three, four, or five Chlamydia trachomatis antigens from the third antigen group and three, four or five Chlamydia trachomatis antigens from the fourth antigen group and one, two, three, four or five Chlamydia trachomatis antigens of the fifth antigen group and one, two, three, four, five, six, seven, eight, nine, ten, eleven or twelve antigens of the sixth antigen group.

Still more preferably, component A of the combination consists of five Chlamydia trachomatis antigens from the first antigen group and three, four or five Chlamydia trachomatis antigens from the third antigen group and three, four or five antigens from the fourth antigen group and one, two, three, four, five or six Chlamydia trachomatis antigens of the fifth antigen group and one, two, three, four, five, six, seven, eight, nine, ten, eleven or twelve antigens of the sixth antigen group.

The invention further includes a composition comprising component A of Chlamydia trachomatis antigens in a combination of Chlamydia trachomatis antigens, component A of said combination selected from the group consisting of two, three, four, five, six, seven, eight, nine, ten, eleven, twelve or thirteen Chlamydia trachomatis antigens of the second antigen group and one, two, three, four, five or six Chlamydia trachomatis antigens of the third antigen group and one, two, three, four, five, six, seven, eight or nine antigens of the fourth antigen group. Preferably, component A of the combination is selected from the group consisting of three, four, or five Chlamydia trachomatis antigens from the second antigen group and three, four or five Chlamydia trachomatis from the third antigen group and three, four or five antigens of the fourth antigen group. Still more preferably, component A of the combination consists of five Chlamydia trachomatis antigens from the second antigen group and three, four or five Chlamydia trachomatis antigens of the third antigen group and three, four or five antigens of the fourth antigen group.

There is an upper limit to the number of Chlamydia trachomatis antigens which will be in the compositions of the invention. Preferably, the number of Chlamydia trachomatis antigens in a composition of the invention is less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3. Still more preferably, the number of Chlamydia trachomatis antigens in a composition of the invention is less than 6, less than 5, or less than 4. The Chlamydia trachomatis antigens used in the invention are preferably isolated, i.e., separate and discrete, from the whole organism with which the molecule is found in nature or, when the polynucleotide or polypeptide is not found in nature, is sufficiently free of other biological macromolecules so that the polynucleotide or polypeptide can be used for its intended purpose.

Preferably, component A of the composition of the present invention comprises a combination of Chlamydia trachomatis antigens, wherein said combination selected from the group consisting of: (1) CT016 and CT128 and CT671 and CT262; (2) CT296 and CT372 and CT635 and CT859; (3) CT412 and CT480 and CT869 and CT871; (4) CT050 and CT153 and CT157 and CT165; (5) CT276 and CT296 and CT456 and CT480; (6) CT089 and CT381 and CT396 and CT548; (7) CT635 and CT700 and CT711 and CT859; (8) CT812 and CT869 and CT552 and CT671; (9) CT713 and CT017 and CT043 and CT082; (10) CT266 and CT443 and CT559 and CT597; and (11) CT045 and CT089 and CT396 and CT398 and CT39 (12) CT681 and CT547; (13) CT623 and CT414; or other combinations thereof.

Preferably, component A of the composition of the present invention comprises a combination of Chlamydia trachomatis antigens, said combination selected from the group consisting of: (1) CT016 and CT128 and CT671 and CT262; (2) CT296 and CT372 and CT635 and CT859; (3) CT412 and CT480 and CT869 and CT871; (4) CT050 and CT153 and CT157 and CT165; (5) CT276 and CT296 and CT456 and CT480; (6) CT089 and CT381 and CT396 and CT548; (7) CT635 and CT700 and CT711 and CT859; (8) CT812 and CT869 and CT552 and CT671; (9) CT713 and CT017 and CT043 and CT082; (10) CT266 and CT443 and CT559 and CT597; and (11) CT045 and CT089 and CT396 and CT398 and CT39 (12) CT681 and CT547; (13) CT623 and CT414; or other combinations thereof; in combination with an immunoregulatory agent which is selected from the group consisting of CFA, Alum, CpG, AlOH, Alum and CpG, AlOH and CpG, LTK63 and LTK63 and CpG.

Preferably, component A of the composition of the present invention comprises a combination of Chlamydia trachomatis antigens, said combination selected from the group consisting of: 1) CT016 and CT128 and CT671 and CT262; (2) CT296 and CT372 and CT635 and CT859; (3) CT412 and CT480 and CT869 and CT871; (4) CT050 and CT153 and CT157 and CT165; (5) CT276 and CT296 and CT456 and CT480; (6) CT089 and CT381 and CT396 and CT548; (7) CT635 and CT700 and CT711 and CT859; (8) CT812 and CT869 and CT552 and CT671; (9) CT713 and CT017 and CT043 and CT082; (10) CT266 and CT443 and CT559 and CT597; and (11) CT045 and CT089 and CT396 and CT398 and CT39 (12) CT681 and CT547; (13) CT623 and CT414; or other combinations thereof; in combination with Alum and CpG or AlOH and CpG.

Preferably, component A of the composition of the present invention comprises a combination of Chlamydia trachomatis antigens, said combination selected from the group consisting of (1) CT242 and CT316; (2) CT467 and CT444; and (3) CT812 and CT082; or other combinations thereof.

Preferably, component A of the composition of the present invention comprises a combination of Chlamydia trachomatis antigens, said combination selected from the group consisting of (1) CT242 and CT316; (2) CT467 and CT444; and (3) CT812 and CT082; or other combinations thereof in combination with an immunoregulatory agent which is selected from the group consisting of CFA, Alum, CpG, AlOH, Alum and CpG, AlOH and CpG, LTK63 and LTK63 and CpG.

Preferably, component A of the composition of the present invention comprises a combination of Chlamydia trachomatis antigens, said combination selected from the group consisting of (1) CT242 and CT316; (2) CT467 and CT444; and (3) CT812 and CT082; or other combinations thereof in combination with Alum and CpG or AlOH and CpG.

Component A of the immunogenic compositions of the present invention may comprise one or more antigens selected from a “fourth antigen” group consisting of: (1) CT559 (YscJ); (2) CT600 (Pal); (3) CT541 (Mip); (4) CT623 (CHLPN 76 kDA homologue) (5) CT700 (Hypothetical protein). (6) CT266 (Hypothetical protein); (7) CT077 (Hypothetical protein); (8) CT456 (Hypothetical protein); (9) CT165 (Hypothetical protein) and (10) CT713 (PorB).

Preferably, component A of the immunogenic compositions of the present invention comprise one or more antigens selected from a “fourth antigen” group consisting of: (1) CT559 (YscJ); (2) CT600 (Pal); (3) CT541 (Mip); (4) CT623 (CHLPN 76 kDA homologue) (5) CT700 (Hypothetical protein). (6) CT266 (Hypothetical protein); (7) CT077 (Hypothetical protein); (8) CT456 (Hypothetical protein); (9) CT165 (Hypothetical protein) and (10) CT713 (PorB); or other combinations thereof in combination with an immunoregulatory agent which is selected from the group consisting of CFA, Alum, CpG, AlOH, Alum and CpG, AlOH and CpG LTK63 and LTK63 and CpG.

Still more preferably, component A of the immunogenic compositions of the present invention comprise one or more antigens selected from a “fourth antigen” group consisting of (1) CT559 (YscJ); (2) CT600 (Pal); (3) CT541 (Mip); (4) CT623 (CHLPN 76 kDA homologue) (5) CT700 (Hypothetical protein). (6) CT266 (Hypothetical protein); (7) CT077 (Hypothetical protein); (8) CT456 (Hypothetical protein); (9) CT165 (Hypothetical protein) and (10) CT713 (PorB); or other combinations thereof in combination with Alum and CpG or AlOH and CpG.

Component A of the immunogenic compositions of the present invention may comprise one or more antigens selected from a “fifth antigen” group consisting of: (1) CT082 (hypothetical); (2) CT181 (Hypothetical); (3) CT050 (Hypothetical); (4) CT157 (Phospholipase D superfamily); and (5) CT128 (AdK adenylate cyclase).

Preferably, component A of the immunogenic compositions of the present invention comprise one or more antigens selected from a “fifth antigen” group consisting of: (1) CT082 (hypothetical); (2) CT181 (Hypothetical); (3) CT050 (Hypothetical); (4) CT157 (Phospholipase D superfamily); and (5) CT128 (AdK adenylate cyclase) or other combinations thereof in combination with an immunoregulatory agent which is selected from the group consisting of CFA, Alum, CpG, AlOH, Alum and CpG, AlOH and CpG, LTK63, LTK63 and CpG.

Still more preferably, component A of the immunogenic compositions of the present invention comprise one or more antigens selected from a “fifth antigen” group consisting of (1) CT082 (hypothetical); (2) CT181 (Hypothetical); (3) CT050 (Hypothetical); (4) CT157 (Phospholipase D superfamily); and (5) CT128 (AdK adenylate cyclase); or other combinations thereof in combination with Alum and CpG or AlOH and CpG.

Component A of the immunogenic compositions of the present invention may comprise one or more antigens selected from a “sixth antigen” group consisting of: (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE); or other combinations thereof.

Preferably, component A of the immunogenic compositions of the present invention comprise one or more antigens selected from a “sixth antigen” group consisting of: (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE); or other combinations thereof in combination with an immunoregulatory agent which is selected from the group consisting of CFA, Alum, CpG, AlOH, Alum and CpG, AlOH and CpG, LTK63, LTK63 and CpG.

Still more preferably, component A of the immunogenic compositions of the present invention comprise one or more antigens selected from a “sixth antigen” group consisting of: (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE); or other combinations thereof in combination with Alum and CpG or AlOH and CpG.

FACS analyses, Western Blot analyses and In-vitro neutralization analyses—carried out as described in the Examples and in WO 03/049762—demonstrate that proteins in the first, second, third, fourth, fifth and antigen groups are surface-exposed and immunoaccessible proteins and are useful immunogens. These properties are not evident from the sequence alone. In addition, proteins described in the fourth, fifth and sixth antigen groups (as well as the first, second, third and fourth antigen groups) which are described as “hypothetical” typically have no known cellular location or biological function and generally, do not have any bacterial homologue, such as a Chlamydia pneumoniae homologues.

Component A of the immunogenic compositions of the present invention may comprise one or more antigens selected from a “third antigen” group consisting of: (1) Pgp3; (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); (10) PmpI; (11) CT681 (MOMP); (12) CT529 (Cap1); (13) Hsp-60; and (14) CT443 (OmcB).

Preferably, component A of the immunogenic compositions of the present invention comprise one or more antigens selected from a “third antigen” group consisting of: (1) Pgp3; (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); (10) PmpI; (11) CT681 (MOMP); (12) CT529 (Cap1); (13) Hsp-60; and (14) CT443 (OmcB); in combination with an immunoregulatory agent which is selected from the group consisting of CFA, Alum, CpG, AlOH, Alum and CpG, AlOH and CpG, LTK63 and LTK63 and CpG.

Still more preferably, component A of the immunogenic compositions of the present invention comprise one or more antigens selected from a “third antigen” group consisting of: (1) Pgp3; (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); (10) PmpI; (11) CT681 (MOMP); (12) CT529 (Cap1); (13) Hsp-60; (14) CT443 (OmcB); in combination with Alum and CpG or AlOH and CpG.

Component A of the immunogenic compositions of the present invention may comprise the Pmp antigens: (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); and (10) PmpI.

Preferably, component A of the immunogenic compositions of the present invention comprise the PmP antigens (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); and (10) PmpI in combination with an immunoregulatory agent which is selected from the group consisting of CFA, Alum, CpG, AlOH, Alum and CpG, AlOH and CpG, LTK63 and LTK63 and CpG.

Still more preferably, component A of the immunogenic compositions of the present invention comprise the PmP antigens (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); and (10) PmpI; in combination with Alum and CpG or AlOH and CpG.

Component A of the immunogenic compositions of the present invention may comprise one or more antigens selected from a “first or second antigen” group consisting of: (1) 045 (PepA); (2) CT089 (LcrE); (3) CT396 (DnaK); (4) CT398 (Hypothetical); (5) CT381 (ArtJ); (6) CT242 (OmpH-like); (7) CT316 (L7/L12); (8) CT444 (OmcA); (9) CT467 (AtoS); (10) CT547 (Hypothetical); (11) CT587 (Enolase); (12) CT823 (HtrA); (13) CT761 (MurG).

Preferably, component A of the immunogenic compositions of the present invention comprise one or more antigens selected from a “first or second antigen” group consisting of: (1) 045 (PepA); (2) CT089 (LcrE); (3) CT396 (DnaK); (4) CT398 (Hypothetical); (5) CT381 (ArtJ); (6) CT242 (OmpH-like); (7) CT316 (L7/L12); (8) CT444 (OmcA); (9) CT467 (AtoS); (10) CT547 (Hypothetical); (11) CT587 (Enolase); (12) CT823 (HtrA); (13) CT761 (MurG); in combination with an immunoregulatory agent which is selected from the group consisting of CFA, Alum, CpG, AlOH, Alum and CpG, AlOH and CpG, LTK63 and LTK63 and CpG.

Still more preferably, component A of the immunogenic compositions of the present invention comprise one or more antigens selected from a “first or second antigen” group consisting of: (1) 045 (PepA); (2) CT089 (LcrE); (3) CT396 (DnaK); (4) CT398 (Hypothetical); (5) CT381 (ArtJ); (6) CT242 (OmpH-like); (7) CT316 (L7/L12); (8) CT444 (OmcA); (9) CT467 (AtoS); (10) CT547 (Hypothetical); (11) CT587 (Enolase); (12) CT823 (HtrA); (13) CT761 (MurG in combination with Alum and CpG or AlOH and CpG.

Preferably, component A of the immunogenic composition comprises: CT089 and CT381 and CT396 and CT548.

Preferably, component A of the immunogenic composition comprises: CT089 and CT381 and CT396 and CT548 in combination with an immunoregulatory agent which is selected from the group consisting of CFA, Alum, CpG, AlOH, Alum and CpG, AlOH and CpG, LTK63 and LTK63 and CpG.

Preferably, component A of the immunogenic composition comprises: CT089 and CT381 and CT396 and CT548 in combination with Alum and CpG or AlOH and CpG

Preferably, component A of the immunogenic compositions of the present invention comprises: CT045 in combination with Alum and CpG or AlOH and CpG.

Preferably, component A of the immunogenic compositions of the present invention comprises: CT089 in combination with Alum and CpG or AlOH and CpG.

Preferably, component A of the immunogenic compositions of the present invention comprises: CT396 combination with Alum and CpG or AlOH and CpG.

Preferably, component A of the immunogenic compositions of the present invention comprises: CT398 in combination with Alum and CpG or AlOH and CpG.

Preferably, component A of the immunogenic compositions of the present invention comprises: CT381 in combination with Alum and CpG or AlOH and CpG.

Preferably, component A of the immunogenic compositions of the present invention comprises: CT242 in combination with Alum and CpG or AlOH and CpG.

Preferably, component A of the immunogenic compositions of the present invention comprises: CT316 in combination with Alum and CpG or AlOH and CpG.

Preferably, component A of the immunogenic compositions of the present invention comprises: CT444 in combination with Alum and CpG or AlOH and CpG.

Preferably, component A of the immunogenic compositions of the present invention comprises: CT467 in combination with Alum and CpG or AlOH and CpG.

Preferably, component A of the immunogenic compositions of the present invention comprises: CT587 in combination with Alum and CpG or AlOH and CpG.

Preferably, component A of the immunogenic compositions of the present invention comprises: CT823 in combination with Alum and CpG or AlOH and CpG.

Preferably, component A of the immunogenic compositions of the present invention comprises: CT761 in combination with Alum and CpG or AlOH and CpG.

### Component B

In one embodiment, the invention provides a composition comprising component B, component B comprising a combination of Chlamydia trachomatis antigens that elicit at least a C. trachomatis specific TH1 immune response. As an example, component B of the combination of Chlamydia trachomatis antigens may include at least one antigen associated with reticulate bodies (RBs) of Chlamydia trachomatis, including but not limited to antigens expressed, exposed on or translocated into, through or across on the inclusion membrane, antigens expressed, secreted, released or translocated into the cytosol of host cells, or antigens processed or degraded in the cytosol of host cells and/or expressed, exposed or presented on the surface of the host cell.

One Example of an antigen associated with RBs of Chlamydia trachomatis is OmpH_like outer membrane protein (CT242). WO 99/53948 and Bannantine and Rockey (1999) Microbiology 145: 2077-2085 teach that CT242 is an Inclusion Membrane Associated protein in the sense that it is localised to Chlamydia intracellular developmental forms at the margins of growing inclusions.

Another example of an antigen associated with RBs of Chlamydia trachomatis is CT456 (formerly regarded as a hypothetical protein but recently designated as a Translocated Actin-Recruiting Protein (Tarp)). In the identification of CT456 as an RB associated antigen, the FACS K-S score is greater than 10 (i.e., 10.8) but is “non-consistent” on Western Blot indicating the RB association of CT456. CT456 appears to be translocated into the host cell by the chlamydial type III secretion system. Once exposed to the cytoplasm, CT456 is rapidly tyrosine-phosphorylated by unknown mechanisms. Phosphorylated CT456 (also known as Tarp—translocated actin-recruiting phosphoprotein), appears to remain “cytoplasmically exposed on the inclusion membrane on one side of internalized EBs for several hours after entry” (Clifton et al (2004) PNAS 101(27); 10166-10171).

An example of a CT456 Hypothetical Protein is disclosed as SEQ ID NOs. 255 & 256 in WO 03/049762 (GenBank accession number: AAC68056.1 GI:3328889 ‘CT456’; SEQ ID NO: 61 in attached sequence listing). Preferred CT456 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 61; and/or (b) which is a fragment of at least n consecutive amino acids of SEQ ID NO: 61, wherein n is 7 or more (e.g. 8, 10, 12, 14, 16, 18, 20, 25, 30, 35, 40, 50, 60, 70, 80, 90, 100, 150, 200, 250 or more). These CT456 Hypothetical proteins include variants (e.g. allelic variants, homologs, orthologs, paralogs, mutants, etc.) of SEQ ID NO: 61. Preferred fragments of (b) comprise an epitope from SEQ ID NO: 61. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 61. Other fragments omit one or more domains of the protein (e.g. omission of a signal peptide, of a cytoplasmic domain, of a transmembrane domain, or of an extracellular domain). Recently, CT456 has been identified as a tyrosine phosphorylated protein (Tarp) which is localized on the cytoplasmic face of the plasma membrane at the site of attachment of surface-associated Chlamydiae. The phosphorylated Tarp appears to remain cytoplasmically exposed on the inclusion membrane on one side of internalized EBs for several hours after entry. Without wishing to be bound by theory, the finding that CT456 is cytoplasmically located suggests that (the metabolically “dormant”) EBs are primed for the translocation of pre-existing effectors, such as CT456 (Tarp) formed during the developmental cycle, by Type III secretion, into the cytoplasm of the cell (see for example Clifton et al (2004) PNAS 101 (27) 10166-10171).

Preferably component B of the composition of the invention comprises a Type Three (III) Secretion System (TTSS) secreted protein or a fragment thereof.

Preferably the TTSS secreted protein is an Inclusion membrane protein selected from the group consisting of IncA, IncB, IncC, IncD, IncE, IncF, IncG, IncS (see Bannantine et al 1998 Infection and Immunity 66; 6017-6021; Stephens et al (1998) Science 282: 754-759, Kalman et al (1999) Nature Genetics 21; 385-389) and CT456 (disclosed and discussed above as SEQ ID NOs 255 and 256 in WO 03/049762 and as SEQ ID NO: 61 in the attached sequence listing).

Typical Type III Secretion System Proteins (including TTSS apparatus proteins and secreted proteins) are discussed in Rockey et al (2000) Infection and Immunity 68(10); 5473-5479 and Fields and Hackstadt (2000) Molecular Microbiology 38(5); 1048-1060) and include but are not limited to YscS (CT563), YscT (CT564), YscU (CT091), YscV (CT090), YscL (CT561), Ysc J (CT559), LcrH-2, LcrD, YscL (CT561), YscN (CT669), YscR (CT562), ScyD (CT576/CT862), SycE (CT088), lcrH-1 and yscT (CT564).

In slightly more detail: yscC is probable Yop proteins translocation protein C;

YscS (CT563) is yop proteins translocation protein S;

YscT (CT564)

YscU (CT091)

YscV (CT090)

YscL (CT561) is yop proteins translocation protein L;

Ysc J (CT559) is yop proteins translocation lipoprotein protein J;

LcrH-2 is low calcium response protein H-2;

LcrD is low calcium response protein D;

YscL (CT561)

YscN (CT669) is yops secretion ATPase;

YscR (CT562) is yop proteins translocation protein R;

ScyD (CT576/CT862)

SycE (CT088) is secretion chaperone;

lcrH-1 is low calcium response protein H-1;

yscT (CT564) is yop proteins translocation protein T; and

IncA, IncB and IncC are Inclusion proteins A, B and C respectively.

Examples of Chlamydia proteins or fragments thereof which are likely to be surface expressed as MHC-Class I antigens and which have a T-cell stimulating effect are disclosed in Table 1 of WO 03/068811. Particularly preferred epitopes are CH-13 (SEQ ID NO: 62: YVFDRILKV), CH-10 (SEQ ID NO: 63: GLTEEIDYV), CH-7 (SEQ ID NO: 64: YMDNNLFYV), CH-8 (SEQ ID NO: 65: FLTLAWWFI) and their Chlamydia trachomatis counterparts which are OmpA (MOMP) (CT681), YscJ (Yop proteins translocation protein J) (CT559), Yop proteins translocation protein T (CT564), Yop proteins translocation protein T (CT564) respectively. CH-13 is a novel HLA-A2 restricted epitope in the outer membrane protein A (OmpA) which appears to mediate specific lysis of peptide-loaded target cells. CH-7, CH-8 and CH-10 are included in protein T and protein J of the Yersinia outer protein (Yop) translocation system. All of these proteins, being either closely related to the Type III secretion system or with a possible membrane localization, may actually be exposed to proteasomal processing and class-I epitopes may be generated. Table 1(a) in the Examples demonstrates that Ysc J is a FACS positive protein. According to the Yersinia model of the Type Three Secretion (TTS) structure, this protein would be expected to be located in the periplasmic space. It is, however, possible that this part of the protein protrudes through the outer membrane or, perhaps in the Chlamydial TTS structure, this protein plays a different role (see Montigiani et al (2002) Infection and Immunity 70(1); 386-379).

By way of background information, the TTSS is a complex protein secretion and delivery machine or apparatus, which may be located on the Elementary Body (EB) and which allows an organism, such as Chlamydia, to maintain its intracellular niche by injecting proteins, such as bacterial effector proteins (which may act as anti-host virulence determinants) into the cytosol of a eukaryotic cell in order to establish the bacterial infection and to modulate the host cellular functions. These injected proteins (the TTSS effector proteins) can have various effects on the host cell which include but are not limited to manipulating actin and other structural proteins and modification of host cell signal transduction systems. The injected (or translocated) proteins or substrates of the TTTS system may also be processed and presented by MHC-class I molecules.

Not all the proteins secreted by a Type III secretion system are delivered into the host cell or have effector function. For example, several Type III proteins are involved in the secretion process itself, its regulation or the translocation of effector proteins through the host cell membrane. By way of example, CT089, also known as Low Calcium Response Element (LcrE) or CoPn—which is the Chlamydia outer protein homologue of YopN) is thought to act as a peripherally associated regulator that prevents secretion (of possible pre-synthesised proteins) in the absence of proper signals from the host cell. In effect, it “plugs” the terminal end of the secretion system until an inductive signal is received. Although the Elementary Body (EB) is regarded as “metabolically inert”, it has been postulated that the Chlamydial TTS system located on the (EB) is triggered by membrane contact and is capable of releasing pre-formed “payload” proteins. The current hypothesis is that Type Three Secretion System (TTSS) becomes active during the intracellular phase of the chlamydial replicative cycle for the secretion of proteins into the host cell cytoplasm and for the insertion of chlamydial proteins (like the Inc set) into the inclusion membrane that separates the growing chlamydial microcolony from the host cell cytoplasm (see Montigiani et al (2002) Infection and Immunity 70(1); 386-379). By way of example, the LcrE (CT089) protein is thought to act as a kind of “molecular syringe”, injecting effector proteins directly across bacterial and eukaryotic cell membranes into the target cell cytoplasm. As Chlamydia bacteria reside in membrane bound vacuoles termed inclusions, it has also been postulated that the Chlamydia TTSS probably translocates proteins into, across or through the vacuolar membrane (i.e. the inclusion membrane) as well as the plasma membrane (see for example Stephens et al 1998 Science (282); 754-759). Immunoblot analysis indicates that both CopN (CT089) and Scc1 (CT088) are present in both chlamydial developmental forms and whole-culture lysates 20 h after infection. Analysis of infected monolayers by indirect immunofluorescence reveals that CopN localised to both bacteria and the inclusion membrane whereas Scc1 was detected only in chlamydiae. Based upon these observations, it appears that, although CoPn is directly associated with chlamydiae, it can also be secreted and associate with the inclusion membrane (see Fields and Hackstadt (2000) Molecular Microbiology 38(5); 1048-1060).

By way of further example, WO 02/48185 teaches that proteins secreted by Chlamydia, especially Inc proteins, are secreted by Type III “machinery”. At least three TTSS effector proteins, IncA, IncB and IncC have been localised to the inclusion membrane. Although their function is unknown, the Inc proteins (or Inclusion Membrane proteins) are thought to be early effectors in the Chlamydia infection process. Inc proteins have been grouped into a family on two criteria: (i) they have a large (larger than 40 residues) hydrophobic domain and (ii) they localise to the membrane of the inclusion in the host cell. Whereas some Incs are expressed and secreted by 2 hours (early cycle) after infection, the expression of other early and mid cycle Type III specific genes (see below) are not detectable until 6-12 hours (mid cycle). After 16-20 hours, the RBs begin to differentiate into EBs, and by 48-72 hours, the EBs predominate within the inclusion. Host cell lysis results in the release of the EBs to the extracellular space where they can infect more cells. The possible expression of early cycle, mid cycle and late cycle Chlamydia genes has been outlined as follows:

Early cycle: (1.5 h-8 h): yscC, yscS, yscL, yscJ and lcrH-2;

Mid cycle: (12 to 18 h post infection): lcrD, yscN and yscR;

Late cycle: (24 h); lcrE, sycE, lcrH-1 and yscT

Thus, Component B of the present invention may comprise TTSS effector proteins. The TTSS effector proteins as described are associated with the RB form of C. trachomatis and may be identified, for example, using immunofluorescence microscopy (see Bannantine et al, Infection and Immunity 66(12); 6017-6021). Effector antibodies to putative Chlamydial TTSS effector proteins secreted by the TTSS machinery may be micro-injected into host cells at specified time points during C. trachomatis infection (e.g., early, mid or late cycle). Host cell reaction to C. trachomatis (e.g., actin remodeling, inhibition of endosomal maturation, host lipid acquisition, and MHC Class I and Class II molecule downregulation) associated with C. trachomatis entry into host cells is then observed. Based on these temporal observations, TTSS effector proteins (RB-associated C. trachomatis proteins) may be detected.

In one embodiment of the present invention, Component B of the composition of the invention comprises a combination of Chlamydia trachomatis antigens, said combination selected from the group consisting of: (1) CT577, (2) ssb (CT044), (3) lpxD (CT243), (4) CT263, (5) accA (CT265), (6) clpC (CT286), (7) dut (CT292), (8) dksA (CT407), (9) euo (CT446), (10) CT460, (11) CT579, (12) CT610, (13) recA (CT650), (14) kdsA (CT655), (15) CT668, (16) CT691, (17) CT734, (18) CT783, (19) CT858, (20) CT875, (21) ORF5, (22) SEQ ID NO: 83 (DT8), (23) yscN, (24) YscL, (25) sycE, (26) CT560, (27) CT149, (28) CT500, and (29) CT841. These antigens are referred to herein as the “seventh antigen group” and represent RB-specific Chlamydia trachomatis antigens as set forth in WO 02/082091, incorporated herein in its entirety. In addition, component B of the present invention includes peptide epitopes that are likely to be surface presented as MHC Class I antigens and have a T-cell stimulating effect.

Even more preferably, Component B of the composition of the present invention comprises a Chlamydia trachomatis proteins selected from the group consisting of: CT016, CT083, CT079, CT056, CT053, CT671, CT666, CT665, CT567, CT566, CT858, CT863 and CT529. WO 02/48185 teaches that these proteins are Type III Secretion system secreted proteins.

Each of the Chlamydia trachomatis antigens of the seventh antigen group is described in more detail below.

(1) CT577 One example of ‘CT577’ protein is disclosed as GenBank accession number: AAC68179, GI:3329019; SEQ ID NO: 66 in the attached sequence listing).

(2) lpxD (CT243) One example of ‘lpxD’ protein is disclosed as GenBank accession number: AAC67836.1, GI:3328653; ‘CT243’; SEQ ID NO: 67 in the attached sequence listing.

(3) CT263 One example of CT263 protein is disclosed as GenBank accession number: AAC67856.1, GI:3328675; ‘CT263’; SEQ ID NO: 68 in the attached sequence listing. Predicted T-cell epitopes of CT263 are identified in WO 02/082091 as SEQ ID NO: 69 KLAEAIFPI, SEQ ID NO: 70 FLKNNKVKL, SEQ ID NO: 71 ALSPPPSGY, SEQ ID NO: 72 FIAKQASLV, SEQ ID NO: 73 TLSLFPFSL, SEQ ID NO: 74 SLVACPCSM, SEQ ID NO: 75 LIFADPAEA, SEQ ID NO: 76 LLLIFADPA, SEQ ID NO: 77 RLEEVSQKL, SEQ ID NO: 78 LTTDTPPVL, SEQ ID NO: 79 KLLDMEGYA, SEQ ID NO: 80 VLSEDPPYI, SEQ ID NO: 81 ALQSYCQAY, SEQ ID NO: 82 KLTQTLVEL, SEQ ID NO: 83 FVGACSPEI, SEQ ID NO: 84 NLTTDTPPV, and SEQ ID NO: 85 LMERAIPPK.

(4) accA (CT265) One example of ‘accA’ protein is disclosed as SEQ ID NOs: 129 & 130 in WO 03/049762 (GenBank accession number: AAC67858.1, GI:3328677; ‘CT265’; SEQ ID NO: 86 in the attached sequence listing.

(5) clpC (CT286) One example of ‘clpC’ protein is disclosed as GenBank accession number: AAC67879.1, GI:3328700; ‘CT286’; SEQ ID NO: 87 in the attached sequence listing.

(6) dut (CT292) One example of ‘dut’ protein is disclosed as GenBank accession number: AAC67885.1, GI:3328706; ‘CT292’; SEQ ID NO: 88 in the attached sequence listing.

(7) dksA (CT407) One example of ‘dksA’ protein is disclosed as GenBank accession number: AAC68004.1, GI:3328835; ‘CT407’; SEQ ID NO: 89 in the attached sequence listing.

(8) euo (CT446) One example of ‘euo’ protein is disclosed as GenBank accession number: AAC68045.1, GI:3328878; ‘CT446’; SEQ ID NO: 90 in the attached sequence listing.

(9) CT460 One example of CT460 protein is disclosed as GenBank accession number: AAC68060, GI:3328894; ‘CT460’; SEQ ID NO: 91 in the attached sequence listing.

(10) CT579 One example of ‘CT579’ protein is disclosed as GenBank accession number: AAC68181, GI:3329021; SEQ ID NO: 92 in the attached sequence listing.

(11) CT610 One example of CT610 protein is disclosed as GenBank accession number: AAC68213, GI:3329055; ‘CT610’; SEQ ID NO: 93 in the attached sequence listing.

(12) recA (CT650) One example of ‘recA’ protein is disclosed as GenBank accession number: AAC68827, GI:3329099; ‘CT650’; SEQ ID NO: 94 in the attached sequence listing.

(13) kdsA (CT655) One example of ‘kdsA’ protein is disclosed as GenBank accession number: AAC68250, GI:3329105; ‘CT655’; SEQ ID NO: 95 in the attached sequence listing.

(14) CT668 One example of CT668 protein is disclosed as GenBank accession number: AAC68263, GI:3329119; ‘CT668’; SEQ ID NO: 96 in the attached sequence listing.

(15) CT691 One example of CT691 protein is disclosed as GenBank accession number: AAC68286, GI:3329144; ‘CT691’; SEQ ID NO: 97 in the attached sequence listing. Predicted T-cell epitopes of CT691 are identified in WO 02/082091 as SEQ ID NO: 98 LLQRELMKV, SEQ ID NO: 99 STINVLFPL, SEQ ID NO: 100 PLQAHLELV, SEQ ID NO: 101 SLFGQSPFA, SEQ ID NO: 102 KLAYRVSMT, SEQ ID NO: 103 VLWMQIIKG, SEQ ID NO: 104 VLFPLFSAL, SEQ ID NO: 105 FLQKTVQSF, SEQ ID NO: 106 FGQSPFAPL, SEQ ID NO: 107 YMLPIFTAL, SEQ ID NO: 108 LLHEFNQLL, SEQ ID NO: 109 VLQRELMQI, SEQ ID NO: 110 PLQAHLEMV, SEQ ID NO: 111 RLFGQSPFA, SEQ ID NO: 112 GLFMPISRA, SEQ ID NO: 113 KLAHRINMT, SEQ ID NO: 114 YLWLQVIRR, SEQ ID NO: 115 TLLHEFNQL and SEQ ID NO: 116 FGQSPFAPL.

(16) CT734 One example of CT734 protein is disclosed as GenBank accession number: AAC68329, GI:3329192; ‘CT734’; SEQ ID NO: 117 in the attached sequence listing.

(17) CT783 One example of CT783 protein is disclosed as GenBank accession number: AAC68378, GI:3329248; ‘CT783’; SEQ ID NO: 118 in the attached sequence listing.

(18) CT858 One example of CT858 protein is disclosed as GenBank accession number: AAC68456, GI:6578188; ‘CT858’; SEQ ID NO: 119 in the attached sequence listing. Predicted T-cell epitopes of CT858 are identified in WO 02/082091 as SEQ ID NO: 120 VLADFIGGL, SEQ ID NO: 121 RMASLGHKV, SEQ ID NO: 122 GLNDFHAGV, SEQ ID NO: 123 FSCADFFPV, SEQ ID NO: 124 MLTDRPLEL, SEQ ID NO: 125 LLENVDTNV, SEQ ID NO: 126 RMILTQDEV, SEQ ID NO: 127 SCADFFPVV, SEQ ID NO: 128 FVFNVQFPN, SEQ ID NO: 129 YLYALLSML, SEQ ID NO: 130 SLAVREHGA, SEQ ID NO: 131 YLPYTVQKS, SEQ ID NO: 132 ATIAPSIRA, SEQ ID NO: 133 LLEVDGAPV, SEQ ID NO: 134 RTAGAGGFV, and SEQ ID NO: 135 SLFYSPMVP.

(19) CT875 One example of CT875 protein is disclosed as GenBank accession number: AAC68473, GI:3329351; ‘CT875’; SEQ ID NO: 136 in the attached sequence listing.

(20) ORF5 One example of ‘ORF5’ protein is disclosed as GenBank accession number: AAB02589, GI:1124829; SEQ ID NO: 137 in the attached sequence listing.

(21) SEQ ID NO: 83 (DT8) One example of the Chlamydia trachomatis secreted protein is disclosed as SEQ ID NO: 1 in WO 02/082091 (SEQ ID NO: 138 in the attached sequence listing).

(22) YscN One example of ‘yscN’ protein is disclosed as GenBank accession number: AAC68264, GI:3329120; SEQ ID NO: 139 in the attached sequence listing.

(23) yscL One example of ‘yscL’ protein is disclosed as GenBank accession number: AAC68163, GI:3329002; SEQ ID NO: 140 in the attached sequence listing.

(24) SycE One example of ‘SycE’ protein is disclosed as GenBank accession number: AAC67679, GI:3328484; SEQ ID NO: 141 in the attached sequence listing.

(25) CT560 One example of ‘CT560’ protein is disclosed as GenBank accession number: AAC68162, GI:3329001; SEQ ID NO: 142 in the attached sequence listing.

(26) CT149 One example of ‘CT149’ protein is disclosed as GenBank accession number: AAC67740, GI:3328551; SEQ ID NO: 143 in the attached sequence listing. Predicted T-cell epitopes of CT149 are identified in WO 02/082091 as SEQ ID NO: 144 FLGAAPAQM, SEQ ID NO: 145 FLGIQDHIL, SEQ ID NO: 146 LLTANGIAV, SEQ ID NO: 147 SLPRRIPVL, SEQ ID NO: 148 GLQEHCRGV, SEQ ID NO: 149 SLGCHTTIH, SEQ ID NO: 150 ILTHFQSNL, SEQ ID NO: 151 VLSCGYNLV, SEQ ID NO: 152 LLKEICATI, SEQ ID NO: 153 RLFLGAAPA, SEQ ID NO: 154 ATVAKYPEV, SEQ ID NO: 155 LLSGSGFAA, SEQ ID NO: 156 LTANGIAVA, and SEQ ID NO: 157 SGFAAPVEV.

(27) CT500 One example of ‘CT500’ protein is disclosed as GenBank accession number: AAC68101, GI:3328937; SEQ ID NO: 158 in the attached sequence listing. Predicted T-cell epitopes of CT500 are identified in WO 02/082091 as SEQ ID NO: 159 FMISGPVVV, SEQ ID NO: 160 ALFGESIGV, SEQ ID NO: 161 SLENAAIEV, SEQ ID NO: 162 LMGATNPKE, and SEQ ID NO: 163 RIAAMKMVH.

(28) CT841 One example of ‘CT841’ protein is disclosed as GenBank accession number: AAC68438, GI:3329313; SEQ ID NO: 164 in the attached sequence listing. Predicted T-cell epitopes of CT841 are identified in WO 02/082091 as SEQ ID NO: 165 LLFGVIFGV, SEQ ID NO: 166 LLAKGQNKV, SEQ ID NO: 167 FTFMPIILV, SEQ ID NO: 168 FLGDVSSGA, SEQ ID NO: 169 LLDAAYQRA, SEQ ID NO: 170 GMSDHLGTV, SEQ ID NO: 171 SLGATHFLP, SEQ ID NO: 172 NLAALENRV, SEQ ID NO: 173 YLFTFMPII, SEQ ID NO: 174 FPTAFFFLL, SEQ ID NO: 175 ILMAATNRP, SEQ ID NO: 176 KTALNDNLV, SEQ ID NO: 177 LLNEAALLA, SEQ ID NO: 178 ELYDQLAVL, SEQ ID NO: 179 ALEKQDPEV, SEQ ID NO: 180 SLGGRIPKG, SEQ ID NO: 181 FMPIILVLL, SEQ ID NO: 182 LLAARKDRT, SEQ ID NO: 183 VTFADVAGI, SEQ ID NO: 184 YTISPRTDV and SEQ ID NO: 185 LIGAPGTGK.

(29) CT044 One example of CT044 protein is disclosed as AAC67635.1, GI:3328436; ‘CT044’; SEQ ID NO: 186 in the attached sequence listing.

Preferred proteins for each of the above C. trachomatis proteins include an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to each of the respective proteins or SEQ ID NOs; and/or (b) which is a fragment of at least n consecutive amino acids of the respective protein and corresponding SEQ ID NO, wherein n is 7 or more (e.g. 8, 9, 10, 12, 14, 16, 18, 20, 25, 30, 35, 40, 50 or more). These preferred proteins may include variants (e.g. allelic variants, homologs, orthologs, paralogs, mutants, etc.) of the respective proteins or SEQ ID NOs. Preferred fragments of (b) comprise an epitope from the respective protein or SEQ ID NO. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of the respective protein or SEQ ID NO. Other fragments omit one or more domains of the protein (e.g. omission of a signal peptide, of a cytoplasmic domain, of a transmembrane domain, or of an extracellular domain).

Preferably, the composition of the invention comprises at least one Chlamydia trachomatis antigen of component B in combination with at least one Chlamydia trachomatis antigen of component A.

Even more preferably, the composition of the invention comprises at least one Chlamydia trachomatis antigen of component B in combination with any combination of Chlamydia trachomatis antigens of component A set forth above.

Still more preferably, the composition of the present invention comprises any Chlamydia trachomatis antigen or combination of Chlamydia trachomatis antigens of component B in combination with any Chlamydia trachomatis antigen or combination of Chlamydia trachomatis antigens of component A (e.g., as set forth above) in combination with an immunoregulatory agent which is selected from the group consisting of CFA, Alum, CpG, AlOH, Alum and CpG, AlOH and CpG, LTK63 and LTK63 and CpG.

Still more preferably the immunogenic compositions of the present invention comprise any Chlamydia trachomatis antigen or combination of Chlamydia trachomatis antigens of component B in combination with any Chlamydia trachomatis antigen or combination of Chlamydia trachomatis antigens of component A (e.g., as set forth above) in combination with Alum and CpG or AlOH and CpG.

### Fusion Proteins

The Chlamydia trachomatis antigens used in the invention may be present in the composition as individual separate polypeptides. Generally, the recombinant fusion proteins of the present invention are prepared as a GST-fusion protein and/or a His-tagged fusion protein.

However, preferably, at least two (i.e. 2, 3, 4, 5, 6, 7, 8, 9, 10, 11, 12, 13, 14, 15, 16, 17, 18, 19 or 20) of the antigens are expressed as a single polypeptide chain (a ‘hybrid’ polypeptide). Hybrid polypeptides offer two principal advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful.

The hybrid polypeptide may comprise two or more polypeptide sequences from the first antigen group. Accordingly, the invention includes a composition comprising a first amino acid sequence and a second amino acid sequence, wherein said first and second amino acid sequences are selected from a Chlamydia trachomatis antigen or a fragment thereof of the first antigen group. Preferably, the first and second amino acid sequences in the hybrid polypeptide comprise different epitopes.

The hybrid polypeptide may comprise two or more polypeptide sequences from the second antigen group. Accordingly, the invention includes a composition comprising a first amino acid sequence and a second amino acid sequence, wherein said first and second amino acid sequences are selected from a Chlamydia trachomatis antigen or a fragment thereof of the second antigen group. Preferably, the first and second amino acid sequences in the hybrid polypeptide comprise difference epitopes.

The hybrid polypeptide may comprise one or more polypeptide sequences from the first antigen group and one or more polypeptide sequences from the second antigen group. Accordingly, the invention includes a composition comprising a first amino acid sequence and a second amino acid sequence, said first amino acid sequence selected from a Chlamydia trachomatis antigen or a fragment thereof from the first antigen group and said second amino acid sequence selected from a Chlamydia trachomatis antigen or a fragment thereof from the second antigen group. Preferably, the first and second amino acid sequences in the hybrid polypeptide comprise difference epitopes.

The hybrid polypeptide may comprise one or more polypeptide sequences from the first antigen group and one or more polypeptide sequences from the third antigen group. Accordingly, the invention includes a composition comprising a first amino acid sequence and a second amino acid sequence, said first amino acid sequence selected from a Chlamydia trachomatis antigen or a fragment thereof from the first antigen group and said second amino acid sequence selected from a Chlamydia trachomatis antigen or a fragment thereof from the third antigen group. Preferably, the first and second amino acid sequences in the hybrid polypeptide comprise difference epitopes.

The hybrid polypeptide may comprise one or more polypeptide sequences from the second antigen group and one or more polypeptide sequences from the third antigen group. Accordingly, the invention includes a composition comprising a first amino acid sequence and a second amino acid sequence, said first amino acid sequence selected from a Chlamydia trachomatis antigen or a fragment thereof from the second antigen group and said second amino acid sequence selected from a Chlamydia trachomatis antigen or a fragment thereof from the third antigen group. Preferably, the first and second amino acid sequences in the hybrid polypeptide comprise difference epitopes.

Hybrids consisting of amino acid sequences from two, three, four, five, six, seven, eight, nine, or ten Chlamydia trachomatis antigens are preferred. In particular, hybrids consisting of amino acid sequences from two, three, four, or five Chlamydia trachomatis antigens are preferred.

Different hybrid polypeptides may be mixed together in a single formulation. Within such combinations, a Chlamydia trachomatis antigen may be present in more than one hybrid polypeptide and/or as a non-hybrid polypeptide. It is preferred, however, that an antigen is present either as a hybrid or as a non-hybrid, but not as both.

Two-antigen hybrids for use in the invention may comprise: (1) PepA & LcrE; (2) PepA & OmpH-like; (3) PepA & L7/L12; (4) PepA & ArtJ; (5) PepA & DnaK; (6) PepA & CT398; (7) PepA & OmcA; (8) PepA & AtoS; (9) PepA & CT547; (10) PepA & Eno; (11) PepA & HrtA; (12) PepA & MurG; (13) LcrE & OmpH-like; (14) LcrE & L7/L12; (15) LcrE & ArtJ; (16) LcrE & DnaK; (17) LcrE & CT398; (18) LcrE & OmcA; (19) LcrE & AtoS; (20) LcrE & CT547; (21) LcrE & Eno; (22) LcrE & HrtA; (23) LcrE & MurG; (24) OmpH-like & L7/L12; (25) OmpH-like & ArtJ; (26) OmpH-like & DnaK; (27) OmpH-like & CT398; (28) OmpH-like & OmcA; (29) OmpH-like & AtoS; (30) OmpH-like & CT547; (31) OmpH-like & Eno; (32) OmpH-like & HrtA; (33) OmpH-like & MurG; (34) L7/L12 & ArtJ; (35) L7/L12 & DnaK; (36) L7/L12 & CT398; (37) L7/L12 & OmcA; (38) L7/L12 & AtoS; (39) L7/L12 & CT547; (40) L7/L12 & Eno; (41) L7/L12 & HrtA; (42) L7/L12 & MurG; (43) ArtJ & DnaK; (44) ArtJ & CT398; (45) ArtJ & OmcA; (46) ArtJ & AtoS; (47) ArtJ & CT547; (48) ArtJ & Eno; (49) ArtJ & HrtA; (50) ArtJ & MurG; (51) DnaK & CT398; (52) DnaK & OmcA; (53) DnaK & AtoS; (54) DnaK & CT547; (55) DnaK & Eno; (56) DnaK & HrtA; (57) DnaK & MurG; (58) CT398 & OmcA; (59) CT398 & AtoS; (60) CT398 & CT547; (61) CT398 & Eno; (62) CT398 & HrtA; (63) CT398 & MurG; (64) OmcA & AtoS; (65) OmcA & CT547; (66) OmcA & Eno; (67) OmcA & HrtA; (68) OmcA & MurG; (69) AtoS & CT547; (70) AtoS & Eno; (71) AtoS & HrtA; (72) AtoS & MurG; (73) CT547 & Eno; (74) CT547 & HrtA; (75) CT547 & MurG; (76) Eno & HrtA; (77) Eno & MurG; (78) HrtA & MurG or (79) PmpD (CT812) and Hypothetical (CT082).

Two antigen hybrids for use in the present invention may also comprise combinations of antigens selected from the third, fourth, fifth and sixth antigen groups.

Hybrid polypeptides can be represented by the formula NH₂-A-{-X-L-}ₙ-B—COOH, wherein: X is an amino acid sequence of a Chlamydia trachomatis antigen or a fragment thereof from the first antigen group, the second antigen group or the third antigen group; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13, 14 or 15.

If a -X- moiety has a leader peptide sequence in its wild-type form, this may be included or omitted in the hybrid protein. In some embodiments, the leader peptides will be deleted except for that of the -X- moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {-X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG (SEQ ID 1), with the Gly-Ser dipeptide being formed from a BamHI restriction site, thus aiding cloning and manipulation, and the (Gly)₄ tetrapeptide being a typical poly-glycine linker.

-A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

-B- is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art. Most preferably, n is 2 or 3.

The invention also provides nucleic acid encoding hybrid polypeptides of the invention. Furthermore, the invention provides nucleic acid which can hybridise to this nucleic acid, preferably under “high stringency” conditions (e.g. 65° C. in a 0.1×SSC, 0.5% SDS solution).

Polypeptides of the invention can be prepared by various means (e.g. recombinant expression, purification from cell culture, chemical synthesis, etc.) and in various forms (e.g. native, fusions, non-glycosylated, lipidated, etc.). They are preferably prepared in substantially pure form (i.e. substantially free from other chlamydial or host cell proteins).

Nucleic acid according to the invention can be prepared in many ways (e.g. by chemical synthesis, from genomic or cDNA libraries, from the organism itself, etc.) and can take various forms (e.g. single stranded, double stranded, vectors, probes, etc.). They are preferably prepared in substantially pure form (i.e. substantially free from other chlamydial or host cell nucleic acids).

The term “nucleic acid” includes DNA and RNA, and also their analogues, such as those containing modified backbones (e.g. phosphorothioates, etc.), and also peptide nucleic acids (PNA), etc. The invention includes nucleic acid comprising sequences complementary to those described above (e.g. for antisense or probing purposes).

The invention also provides a process for producing a polypeptide of the invention, comprising the step of culturing a host cell transformed with nucleic acid of the invention under conditions which induce polypeptide expression.

The invention provides a process for producing a polypeptide of the invention, comprising the step of synthesising at least part of the polypeptide by chemical means.

The invention provides a process for producing nucleic acid of the invention, comprising the step of amplifying nucleic acid using a primer-based amplification method (e.g. PCR).

The invention provides a process for producing nucleic acid of the invention, comprising the step of synthesising at least part of the nucleic acid by chemical means.

### Strains

Preferred polypeptides of the invention comprise an amino acid sequence found in C. trachomatis serovar D, or in one or more of an epidemiologically prevalent serotype In relation to a genital tract Chlamydia infection, the prevalent serotypes are typically D, E, F, H I and K, more typically, the serovars D, E, I and F, even more typically D, E, F.

Where hybrid polypeptides are used, the individual antigens within the hybrid (i.e. individual -X- moieties) may be from one or more strains. Where n=2, for instance, X₂ may be from the same strain as X₁ or from a different strain. Where n=3, the strains might be (i) X₁=X₂=X₃ (ii) X₁=X₂≠X₃ (iii) X₁≠X₂=X₃ (iv) X₁≠X₂≠X₃ or (v) X₁=X₃≠X₂, etc.

### Heterologous Host

Whilst expression of the polypeptides of the invention may take place in Chlamydia, the invention preferably utilises a heterologous host. The heterologous host may be prokaryotic (e.g. a bacterium) or eukaryotic. It is preferably E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

### Immunogenic Compositions and Medicaments

Compositions of the invention are preferably immunogenic compositions, and are more preferably vaccine compositions. The pH of the composition is preferably between 6 and 8, preferably about 7. The pH may be maintained by the use of a buffer. The composition may be sterile and/or pyrogen-free. The composition may be isotonic with respect to humans.

Vaccines according to the invention may either be prophylactic (i.e. to prevent infection) or therapeutic (i.e. to treat infection), but will typically be prophylactic. Accordingly, the invention includes a method for the therapeutic or prophylactic treatment of Chlamydia trachomatis infection in an animal susceptible to chlamydial infection comprising administering to said animal a therapeutic or prophylactic amount of the immunogenic compositions of the invention. Preferably, the immunogenic composition comprises a combination of Chlamydia trachomatis antigens, said combination selected from the group consisting of two, three, four, or all five Chlamydia trachomatis antigens of the first antigen group and at least one Chlamydia trachomatis antigen of the seventh antigen group. Still more preferably, the combination consists of all five Chlamydia trachomatis antigens of the first antigen group and at least one Chlamydia trachomatis antigen of the seventh antigen group.

Alternatively, the immunogenic composition comprises a combination of Chlamydia trachomatis antigens, said combination selected from the group consisting of two, three, four, five, six, seven, eight, nine, ten, eleven, twelve, or thirteen Chlamydia trachomatis antigens selected from the second antigen group and at least one Chlamydia trachomatis antigen of the seventh antigen group. Preferably, the combination is selected from the group consisting of three, four, or five Chlamydia trachomatis antigens selected from the second antigen group and at least one Chlamydia trachomatis antigen of the seventh antigen group. Still more preferably, the combination consists of five Chlamydia trachomatis antigens selected from the second antigen group and at least one Chlamydia trachomatis antigen of the seventh antigen group.

Alternatively, the immunogenic composition comprises a combination of Chlamydia trachomatis antigens, said combination consisting of two, three, four, or five Chlamydia trachomatis antigens of the first antigen group and one, two, three, four, five or six Chlamydia trachomatis antigens of the third antigen group and at least one Chlamydia trachomatis antigen of the seventh antigen group. Preferably, the combination consists of three, four or five Chlamydia trachomatis antigens of the first antigen group and one, two, three, four, five or six Chlamydia trachomatis antigens of the third antigen group and at least one Chlamydia trachomatis antigen of the seventh antigen group.

Alternatively, the immunogenic composition comprises a combination of Chlamydia trachomatis antigens, said combination consisting of two, three, four, five, six, seven, eight, nine, ten, eleven, twelve or thirteen Chlamydia trachomatis antigens of the second antigen group and one, two, three, four, five or six Chlamydia trachomatis antigens of the third antigen group and at least one Chlamydia trachomatis antigen of the seventh antigen group. Preferably, the combination is selected from the group consisting of three, four, or five Chlamydia trachomatis antigens from the second antigen group and three, four or five Chlamydia trachomatis from the third antigen group and at least one Chlamydia trachomatis antigen of the seventh antigen group. Still more preferably, the combination consists of five Chlamydia trachomatis antigens from the second antigen group and three, four or five Chlamydia trachomatis antigens of the third antigen group and at least one Chlamydia trachomatis antigen of the seventh antigen group.

Alternatively, the immunogenic composition comprises a combination of Chlamydia trachomatis antigens, said combination consisting of two, three, four, five, six, seven, eight, nine or ten Chlamydia trachomatis antigens of the fourth antigen group and one, two, three, four or five Chlamydia trachomatis antigens of the fifth antigen group and one, two, three, four, five, six, seven, eight, nine, ten, eleven, twelve, thirteen, fourteen, fifteen, sixteen, seventeen, eighteen, nineteen, twenty or twentyone antigens of the sixth antigen group and at least one Chlamydia trachomatis antigen of the seventh antigen group. Preferably, the combination is selected from the group consisting of three, four, or five Chlamydia trachomatis antigens from the fourth antigen group and three, four or five Chlamydia trachomatis from the fifth antigen group and at least one Chlamydia trachomatis antigen of the seventh antigen group. Still more preferably, the combination consists of five Chlamydia trachomatis antigens from the fourth antigen group and three, four or five Chlamydia trachomatis antigens of the fifth antigen group and at least one Chlamydia trachomatis antigen of the seventh antigen group.

The invention also comprises an immunogenic composition comprising one or more immunoregulatory agents. Preferably, one or more of the immunoregulatory agents include an adjuvant. The adjuvant may be selected from one or more of the group consisting of a TH1 adjuvant and TH2 adjuvant, further discussed below. The adjuvant may be selected from the group consisting of a mineral salt, such as an aluminium salt and an oligonucleotide containing a CpG motif. Most preferably, the immunogenic composition includes both an aluminium salt and an oligonucleotide containing a CpG motif. Alternatively, the immunogenic composition includes an ADP ribosylating toxin, such as a detoxified ADP ribosylating toxin and an oligonucleotide containing a CpG motif.

The compositions of the invention will preferably elicit both a cell mediated immune response as well as a humoral immune response in order to effectively address a Chlamydia intracellular infection. This immune response will preferably induce long lasting (e.g. neutralising) antibodies and a cell mediated immunity that can quickly respond upon exposure to Chlamydia. Preferably, the immunogenic compositions elicit an antibody response and a cell mediate immune response effective to protect or treat Chlamydia infections associated with Serovars D, E, F and I.

Two types of T cells, CD4 and CD8 cells, are generally thought necessary to initiate and/or enhance cell mediated immunity and humoral immunity CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-gamma, and TNF-beta. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

An enhanced TH1 immune response may include one or more of an increase in CTLs, an increase in one or more of the cytokines associated with a TH1 immune response (such as IL-2, IFN-gamma, and TNF-beta), an increase in activated macrophages, an increase in NK activity, or an increase in the production of IgG2a. Preferably, the enhanced TH1 immune response will include an increase in IgG2a production.

An enhanced TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

As discussed further in the Examples, use of the combination of a mineral salt, such as an aluminium salt, and an oligonucleotide containing a CpG motif provide for an enhanced immune response. This improved immune response is wholly unexpected and could not be predicted from the use of either agent alone. The invention therefore includes an oligonucleotide containing a CpG motif; a mineral salt such as an aluminium salt, and an antigen associated with a sexually transmissible disease, such as a Chlamydia trachomatis antigen. Further examples of antigens associated with a sexually transmissible disease are discussed further below.

The invention also provides a composition of the invention for use as a medicament. The medicament is preferably able to raise an immune response in a mammal (i.e. it is an immunogenic composition) and is more preferably a vaccine. The invention also provides the use of the compositions of the invention in the manufacture of a medicament for raising an immune response in a mammal. The medicament is preferably a vaccine.

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

The invention also provides for a kit comprising a first component comprising a combination of Chlamydia trachomatis antigens. The combination of Chlamydia trachomatis antigens may be one or more of the immunogenic compositions of the invention. The kit may further include a second component comprising one or more of the following: instructions, syringe or other delivery device, adjuvant, or pharmaceutically acceptable formulating solution.

The invention also provides a delivery device pre-filled with the immunogenic compositions of the invention.

The invention also provides a method for raising an immune response in a mammal comprising the step of administering an effective amount of a composition of the invention. The immune response is preferably protective and preferably involves antibodies and/or cell-mediated immunity. Preferably, the immune response includes one or both of a TH1 immune response and a TH2 immune response. The method may raise a booster response.

The mammal is preferably a human. Where the vaccine is for prophylactic use, the human is preferably a child (e.g. a toddler or infant) or a teenager or an adult; where the vaccine is for therapeutic use, the human is preferably a teenager or an adult. A vaccine intended for children may also be administered to adults e.g. to assess safety, dosage, immunogenicity, etc. Preferably, the human is a teenager. More preferably, the human is a pre-adolescent teenager. Even more preferably, the human is a pre-adolescent female or male Preferably the pre-adolescent male or female is around 9-12 years of age.

One way of assessing the immunogenicity of the component proteins of the immunogenic compositions of the present invention is to express the proteins recombinantly and to screen patient sera or mucosal secretions by immunoblot or by protein or DNA microarray. A positive reaction between the protein and the patient serum indicates that the patient has previously mounted an immune response to the protein in question—that is, the protein is an immunogen. This method may also be used to identify immunodominant proteins.

One way of checking efficacy of therapeutic treatment involves monitoring C. trachomatis infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against the Chlamydia trachomatis antigens in the compositions of the invention after administration of the composition. Typically, serum Chlamydia specific antibody responses are determined post-immunization but pre-challenge whereas mucosal Chlamydia specific antibody body responses are determined post-immunization and post-challenge.

These uses and methods are preferably for the prevention and/or treatment of a disease caused by a Chlamydia (e.g. trachoma, pelvic inflammatory disease, epididymitis, infant pneumonia, etc.). The compositions may also be effective against C. pneumoniae.

The vaccine compositions of the present invention can be evaluated in in vitro and in vivo animal models prior to host, e.g., human, administration. For example, in vitro neutralization by Peterson et al (1988) is suitable for testing vaccine compositions directed toward Chlamydia trachomatis.

One example of such an in vitro test is described as follows. Hyper-immune antisera is diluted in PBS containing 5% guinea pig serum, as a complement source. Chlamydia trachomatis (10⁴ IFU; inclusion forming units) are added to the antisera dilutions. The antigen-antibody mixtures are incubated at 37° C. for 45 minutes and inoculated into duplicate confluent Hep-2 or HeLa cell monolayers contained in glass vials (e.g., 15 by 45 mm), which have been washed twice with PBS prior to inoculation. The monolayer cells are infected by centrifugation at 1000×g for 1 hour followed by stationary incubation at 37° C. for 1 hour. Infected monolayers are incubated for 48 or 72 hours, fixed and stained with Chlamydia specific antibody, such as anti-MOMP. Inclusion-bearing cells are counted in ten fields at a magnification of 200×. Neutralization titer is assigned on the dilution that gives 50% inhibition as compared to control monolayers/IFU.

The efficacy of immunogenic compositions can also be determined in vivo by challenging animal models of Chlamydia trachomatis infection, e.g., guinea pigs or mice, with the immunogenic compositions. The immunogenic compositions may or may not be derived from the same serovars as the challenge serovars. Preferably the immunogenic compositions are derivable from the same serovars as the challenge serovars. More preferably, the immunogenic composition and/or the challenge serovar are derivable from the group of genital tract serovars consisting of D, E, F, H, I and K and/or combinations thereof. Even more preferably, the immunogenic composition and/or the challenge serovar are derivable from the group of genital tract serovars consisting of D, E, F and I. Even more preferably, the immunogenic composition and/or the challenge serovar are derivable from the group of genital tract serovars consisting of D, E and F. In women, the serotypes D and F have been associated with asymptomatic infection while serotype E has been associated with both symptomatic and asymptomatic infection. Other possible serovar generally associated with male infections include LGV serovars. The serovars of the present invention are obtainable from clinical isolates or from culture collections such as the American Tissue Culture Collection (ATCC).

In vivo efficacy models include but are not limited to: (i) A murine infection model using human Chlamydia trachomatis serotypes, such as serotypes D, E, F, H, I and K; (ii) a murine disease model which is a murine model using a mouse-adapted Chlamydia trachomatis strain, such as the Chlamydia trachomatis mouse pneumonitis (MoPn) strain also known as Chlamydia muridarum; and (iii) a primate model using human Chlamydia trachomatis isolates. The MoPn strain is a mouse pathogen while human Chlamydia trachomatis serotypes, such as serotypes D, E, F, H, I and K are human pathogens (see for example, Brunham et al (2000) J Infect Dis 181 (Suppl 3) S538-S543; Murdin et al (2000) J Infect Dis 181 (Suppl 3) S544-S551 and Read et al (2000) NAR 28(6); 1397-1406). As the Examples demonstrate, human Chlamydia trachomatis serotypes, such as serovar D can be used in mouse models although they normally require high inocula or pretreatment with progesterone. Progesterone is generally used because it seems to render the genital epithelium more susceptible to chlamydial infection (see Pal et al 2003 Vaccine 21: 1455-1465). One the other hand, MoPn, which was originally isolated from mouse tissues, is thought to be a natural murine pathogen and thus offers an evolutionarily adapted pathogen for analysis of host-pathogen interactions. Although the MoPn serovar is thought to have a high degree of DNA homology to the human Chlamydia serovars, it may also have some unique properties (see for example, Pal et al (2002) Infection and Immunity 70(9); 4812-4817.

By way of example, in vivo vaccine compositions challenge studies can be performed in the murine model of Chlamydia trachomatis (Morrison et al 1995). A description of one example of this type of approach is as follows. Female mice 7 to 12 weeks of age receive 2.5 mg of depoprovera subcutaneously at 10 and 3 days before vaginal infection. Post-vaccination, mice are infected in the genital tract with 1,500 inclusion-forming units of Chlamydia trachomatis contained in 5 ml of sucrose-phosphate-glutamate buffer, pH 7.4. The course of infection is monitored by determining the percentage of inclusion-bearing cells by indirect immunofluorescence with Chlamydia trachomatis specific antisera, or by a Giemsa-stained smear from a scraping from the genital tract of an infected mouse. The presence of antibody titers in the serum of a mouse is determined by an enzyme-linked immunosorbent assay. The immunogenic compositions of the present invention can be administered using a number of different immunization routes such as but not limited to intra-muscularly (i.m.), intra-peritoneal (i.p.), intra-nasal (i.n.), sub-cutaneous (s.c) or transcutaneous (t.c) routes. Generally, any route of administration can be used provided that the desired immune response at the required mucosal surface or surfaces is achieved. Likewise, the challenge serovars may be administered by a number of different routes. Typically, the challenge serovars are administered mucosally, such as but not limited to genital challenge or intra-nasal (i.n) challenge.

Alternative in-vivo efficacy models include guinea pig models. For example, in vivo vaccine composition challenge studies in the guinea pig model of Chlamydia trachomatis infection can be performed. A description of one example of this type of approach follows. Female guinea pigs weighing 450-500 g are housed in an environmentally controlled room with a 12 hour light-dark cycle and immunized with vaccine compositions via a variety of immunization routes. Post-vaccination, guinea pigs are infected in the genital tract with the agent of guinea pig inclusion conjunctivitis (GPIC), which has been grown in HeLa or McCoy cells (Rank et al. (1988)). Each animal receives approximately 1.4×10⁷ inclusion forming units (IFU) contained in 0.05 ml of sucrose-phosphate-glutamate buffer, pH 7.4 (Schacter, 1980). The course of infection monitored by determining the percentage of inclusion-bearing cells by indirect immunofluorescence with GPIC specific antisera, or by Giemsa-stained smear from a scraping from the genital tract (Rank et al 1988). Antibody titers in the serum is determined by an enzyme-linked immunosorbent assay.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal (See e.g. WO99/27961) or transcutaneous (See e.g. WO02/074244 and WO02/064162), intranasal (See e.g. WO03/028760), ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage treatment can be a single dose schedule or a multiple dose schedule. Multiple doses may be used in a primary immunization schedule and/or in a booster immunization schedule. In a multiple dose schedule the various doses may be given by the same or different routes e.g. a parenteral prime and mucosal boost, a mucosal prime and parenteral boost, etc.

Chlamydial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. The immunogenic compositions of the present invention can be used to prevent or alleviate or to treat acute signs of lower genital tract disease, such as cervicitis or urethritis. The immunogenic compositions may also be used to prevent or to alleviate or to treat chronic forms of the disease involving other parts of the genital tract (such as the upper genital tract) or body. In the female, the chronic manifestations of chlamydial infection include Infertility, such as Tubal Factor Infertility (TFI), Pelvic Inflammatory Disease (PID), Fallopian Tube Damage, Chronic Pelvic Pain, Perihepatitis, and Ectopic Pregnancy (EP). In the male, urethritis may be followed by prostatitis. In both sexes arthritis (“Sexually Acquired Reactive Arthritis” or SARA) may occur as a sequel to the primary infection.

### Further Components of the Composition

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in Gennaro (2000) Remington: The Science and Practice of Pharmacy. 20th ed., ISBN: 0683306472.

### ImmunoRegulatory Agents

Vaccines of the present invention may be administered in conjunction with other immunoregulatory agents. In particular, compositions will usually include an adjuvant. Adjuvants for use with the invention include, but are not limited to, one or more of the following set forth below:

### A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO00/23105).

Aluminum salts may be included in immunogenic compositions and/or vaccines of the invention such that the dose of Al³⁺ is between 0.2 and 1.0 mg per dose.

Preferably the adjuvant is alum, preferably an aluminium salt such as aluminium hydroxide (AlOH) or aluminium phosphate or aluminium sulfate. Still more preferably the adjuvant is aluminium hydroxide (AlOH).

Preferably a mineral salt, such as an aluminium salt, is combined with and another adjuvant, such as an oligonucleotide containing a CpG motif or an ADP ribosylating toxin. Still more preferably, the mineral salt is combined with an oligonucleotide containing a CpG motif.

### B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See WO90/14837. See also, Frey et al., “Comparison of the safety, tolerability, and immunogenicity of a MF59-adjuvanted influenza vaccine and a non-adjuvanted influenza vaccine in non-elderly adults”, Vaccine (2003) 21:4234-4237. MF59 is used as the adjuvant in the FLUAD™ influenza virus trivalent subunit vaccine.

Particularly preferred adjuvants for use in the compositions are submicron oil-inwater emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isoglutaminyl-L-alanine-2-(1′-2′-dipalmitoyl-sn-glycero-3-huydroxyphosphosphoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325, incorporated herein by reference in their entireties. Complete Freund's adjuvant (CFA) and incomplete Freund's adjuvant (IFA) may also be used as adjuvants in the invention.

### C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of additional detergent. See WO00/07621.

A review of the development of saponin based adjuvants can be found at Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

### D. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO03/024480, WO03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavirus Virus-Like Particles Are Efficient Oral Immunogens when Coadministered with Escherichia coli Heat-Labile Entertoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16. Immunopotentiating reconstituted influenza virosomes (IRIV) are used as the subunit antigen delivery system in the intranasal trivalent INFLEXAL™ product {Mischler & Metcalfe (2002) Vaccine 20 Suppl 5:B17-23} and the INFLUVAC PLUS™ product.

### E. Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

(2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WO01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658; Bhagat et al., “CpG penta- and hexadeoxyribonucleotides as potent immunomodulatory agents” BBRC (2003) 300:853-861 and WO03/035836.

Preferably the adjuvant is CpG. Even more preferably, the adjuvant is Alum and an oligonucleotide containing a CpG motif or AlOH and an oligonucleotide containing a CpG motif.

(4) ADP-Ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references, each of which is specifically incorporated by reference herein in their entirety: Beignon, et al., “The LTR72 Mutant of Heat-Labile Enterotoxin of Escherichia coli Enhances the Ability of Peptide Antigens to Elicit CD4+ T Cells and Secrete Gamma Interferon after Coapplication onto Bare Skin”, Infection and Immunity (2002) 70(6):3012-3019; Pizza, et al., “Mucosal vaccines: non toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol (2000) 290(4-5):455-461; Scharton-Kersten et al., “Transcutaneous Immunization with Bacterial ADP-Ribosylating Exotoxins, Subunits and Unrelated Adjuvants”, Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol (1995) 15(6):1165-1167, specifically incorporated herein by reference in its entirety.

Preferably the adjuvant is an ADP-ribosylating toxin and an oligonucleotide containing a CpG motif (see for example, WO 01/34185)

Preferably the adjuvant is a detoxified ADP-ribosylating toxin and an oligonucleotide containing a CpG motif.

Preferably the detoxified ADP-ribosylating toxin is LTK63 or LTK72.

Preferably the adjuvant is LTK63. Preferably the adjuvant is LTK72.

Preferably the adjuvant is LTK63 and an oligonucleotide containing a CpG motif.

Preferably the adjuvant is LTK72 and an oligonucleotide containing a CpG motif.

### F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g. WO99/27960.

### G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### H. Liposomes

Examples of liposome formulations suitable for use as adjuvants are described in U.S. Pat. No. 6,090,406, U.S. Pat. No. 5,916,588, and EP 0 626 169.

### L Polyoxyethylene Ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152).

Preferred polyoxyethylene ethers are selected from the following group: polyoxyethylene-9-lauryl ether (laureth 9), polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether.

### J. Polyphosphazene (PCPP)

PCPP formulations are described, for example, in Andrianov et al., “Preparation of hydrogel microspheres by coacervation of aqueous polyphosphazene solutions”, Biomaterials (1998) 19(1-3):109-115 and Payne et al., “Protein Release from Polyphosphazene Matrices”, Adv. Drug. Delivery Review (1998) 31(3):185-196.

### K. Muramyl Peptides

Examples of muramyl peptides suitable for use as adjuvants in the invention include N-acetyl-muramyl-L-threonyl-D-isoglutamine(thr-MDP), N-acetyl-normuramyl-1-alanyl-d-isoglutamine(nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

### L. Imidazoquinolone Compounds

Examples of imidazoquinolone compounds suitable for use adjuvants in the invention include Imiquamod and its homologues, described further in Stanley, “Imiquimod and the imidazoquinolones: mechanism of action and therapeutic potential” Clin Exp Dermatol (2002) 27(7):571-577 and Jones, “Resiquimod 3M”, Curr Opin Investig Drugs (2003) 4(2):214-218.

The invention may also comprise combinations of aspects of one or more of the adjuvants identified above. For example, the following adjuvant compositions may be used in the invention:

(1) a saponin and an oil-in-water emulsion (WO99/11241);
(2) a saponin (e.g., QS21)+a non-toxic LPS derivative (e.g. 3dMPL) (see WO94/00153);
(3) a saponin (e.g., QS21)+a non-toxic LPS derivative (e.g. 3dMPL)+a cholesterol;
(4) a saponin (e.g. QS21)+3dMPL+IL-12 (optionally+a sterol) (WO98/57659);
(5) combinations of 3dMPL with, for example, QS21 and/or oil-in-water emulsions (See European patent applications 0835318, 0735898 and 0761231);
(6) SAF, containing 10% Squalane, 0.4% Tween 80, 5% pluronic-block polymer L121, and thr-MDP, either microfluidized into a submicron emulsion or vortexed to generate a larger particle size emulsion.
(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);
(8) one or more mineral salts (such as an aluminum salt)+a non-toxic derivative of LPS (such as 3dPML); and
(9) one or more mineral salts (such as an aluminum salt)+an immunostimulatory oligonucleotide (such as a nucleotide sequence including a CpG motif).

Aluminum salts and MF59 are preferred adjuvants for use with injectable influenza vaccines. Bacterial toxins and bioadhesives are preferred adjuvants for use with mucosally-delivered vaccines, such as nasal vaccines.

### M. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

### Further Antigens

The compositions of the invention may further comprise antigen derived from one or more sexually transmitted diseases in addition to Chlamydia trachomatis. Preferably the antigen is derived from one or more of the following sexually transmitted diseases: N. gonorrhoeae (See e.g. WO99/24578, WO99/36544, WO99/57280, WO02/079243); human papilloma virus; Treponema pallidum; herpes simplex virus (HSV-1 or HSV-2); HIV (HIV-1 or HIV-2); and Haemophilus ducreyi.

A preferred composition comprises: (1) at least t of the Chlamydia trachomatis antigens from either the first antigen group or the second antigen group, where t is 2, 3, 4, 5, 6, 7, 8, 9, 10, 11, 12 or 13, preferably t is five; (2) one or more antigens from another sexually transmitted disease. Preferably, the sexually transmitted disease is selected from the group consisting of herpes simplex virus, preferably HSV-1 and/or HSV-2; human papillomavirus; N. gonorrhoeae; Treponema pallidum; and Haemophilus ducreyi. These compositions can thus provide protection against the following sexually-transmitted diseases: chlamydia, genital herpes, genital warts, gonorrhoea, syphilis and chancroid (See, WO00/15255).

Antigens associated with or derived from N. gonorrhoeae may include, for example, a Por (or porin) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al., J Infectious Disease (2000) 182:848-855).

Antigens associated with or derived from human papillomavirus (HPV) may include, for example, one or more of E1-E7, L1, L2, and fusions thereof. Preferably, the compositions of the invention may include a virus-like particle (VLP) comprising the L1 major capsid protein. Preferably the HPV antigens are protective against one or more of HPV serotypes 6, 11, 16 and 18.

Where a saccharide or carbohydrate antigen is used, it is preferably conjugated to a carrier protein in order to enhance immunogenicity (See e.g. Ramsay et al. (2001) Lancet 357(9251):195-196; Lindberg (1999) Vaccine 17 Suppl 2:S28-36; Buttery & Moxon (2000) J R Coll Physicians Lond 34:163-168; Ahmad & Chapnick (1999) Infect Dis Clin North Am 13:113-133, viiGoldblatt (1998) J. Med. Microbiol. 47:563-567; European patent 0 477 508; U.S. Pat. No. 5,306,492 International patent application WO98/42721 Conjugate Vaccines (eds. Cruse et al.) ISBN 3805549326, particularly vol. 10:48-114 Hermanson (1996) Bioconjugate Techniques ISBN: 0123423368 or 012342335X). Preferred carrier proteins are bacterial toxins or toxoids, such as diphtheria or tetanus toxoids. The CRM₁₉₇ diphtheria toxoid is particularly preferred (See Research Disclosure, 453077 (January 2002). Other carrier polypeptides include the N. meningitidis outer membrane protein (See EP-A-0372501), synthetic peptides (See EP-A-0378881 and EP-A-0427347), heat shock proteins (See WO93/17712 and WO94/03208), pertussis proteins (See WO98/58668 and EP-A-0471177), protein D from H. influenzae (See WO00/56360), cytokines (See WO91/01146), lymphokines, hormones, growth factors, toxin A or B from C. difficile (See WO00/61761), iron-uptake proteins (See WO01/72337), etc. Where a mixture comprises capsular saccharides from both serogroups A and C, it may be preferred that the ratio (w/w) of MenA saccharide:MenC saccharide is greater than 1 (e.g. 2:1, 3:1, 4:1, 5:1, 10:1 or higher). Different saccharides can be conjugated to the same or different type of carrier protein. Any suitable conjugation reaction can be used, with any suitable linker where necessary.

Toxic protein antigens may be detoxified where necessary e.g. detoxification of pertussis toxin by chemical and/or genetic means.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the composition will typically be present at a concentration of at least 1 μg/ml each.

In general, the concentration of any given antigen will be sufficient to elicit an immune response against that antigen.

As an alternative to using protein antigens in the composition of the invention, nucleic acid encoding the antigen may be used (See e.g. Robinson & Torres (1997) Seminars in Immunology 9:271-283; Donnelly et al. (1997) Annu Rev Immunol 15:617-648 Scott-Taylor & Dalgleish (2000) Expert Opin Investig Drugs 9:471-480 Apostolopoulos & Plebanski (2000) Curr Opin Mol Ther 2:441-447 Ilan (1999) Curr Opin Mol Ther 1:116-120 Dubensky et al. (2000) Mol Med 6:723-732; Robinson & Pertmer (2000) Adv Virus Res 55:1-74 Donnelly et al. (2000) Am J Respir Crit Care Med 162(4 Pt 2):S190-193 Davis (1999) Mt. Sinai J. Med. 66:84-90). Protein components of the compositions of the invention may thus be replaced by nucleic acid (preferably DNA e.g. in the form of a plasmid) that encodes the protein.

The present invention also provides a method of neutralizing C. trachomatis infectivity in a patient in which an effective amount of an immunogenic composition of the present invention is administered to the patient.

In another embodiment of the present invention, a method is provided for immunizing a patient against Chlamydia trachomatis in which an effective amount of an immunogenic composition of the present invention is administered to the patient.

In another embodiment of the present invention, a method is provided for raising antibodies specific for Chlamydia trachomatis elementary bodies comprising administering to a patient an immunogenic composition of the present invention.

In another embodiment of the present invention, a method is provided for raising antibodies which recognize at least one Chlamydia trachomatis antigen of an immunogenic composition of the present invention.

In another embodiment of the present invention, a method is provided for detecting a Chlamydia trachomatis elementary body in a biological sample, comprising contacting the sample with an antibody which recognizes a Chlamydia trachomatis antigen of an immunogenic composition of the present invention.

In another embodiment, the present invention provides a use of an immunogenic composition of the present invention in the manufacture of a medicament for the prevention or treatment of a Chlamydia trachomatis infection.

In still another embodiment, the present invention provides a use of an immunogenic composition of the present invention in the manufacture of a medicament for neutralizing a Chlamydia trachomatis specific infection.

One example of an immunogenic composition of the present invention is a combination of Chlamydia trachomatis antigens comprising at least one Chlamydia trachomatis antigen associated with elementary bodies of Chlamydia trachomatis and at least one Chlamydia trachomatis antigen associated with reticulate bodies of Chlamydia trachomatis. In another example, the immunogenic composition may be a combination of Chlamydia trachomatis antigens comprising at least one Chlamydia trachomatis antigen of a first antigen group and at least one Chlamydia trachomatis antigen of a second antigen group, the first antigen group comprising a Type III secretion system (TTSS) protein and the second antigen group comprising a Type III secretion system (TTSS) effector protein. In yet another example, the immunogenic composition may be a combination of Chlamydia trachomatis antigens comprising at least one Chlamydia trachomatis antigen that is conserved over at least two serovars. In still another example, the immunogenic composition may be a combination of Chlamydia trachomatis antigens eliciting a Chlamydia trachomatis specific TH1 immune response and a Chlamydia trachomatis specific TH2 immune response.

### DEFINITIONS

The term “comprising” means “including” as well as “consisting” e.g. a composition “comprising” X may consist exclusively of X or may include something additional e.g. X+Y.

The term “about” in relation to a numerical value x means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489

### EXAMPLES

The present invention will be defined only by way of example. It will be understood that the invention has been described by way of example only and modifications may be made whilst remaining within the scope and spirit of the invention. Tables 1(a) and 1(b), below, summarize characterization data of the CT antigens of the invention. These tables also include data which will be further explained in the examples which follow.

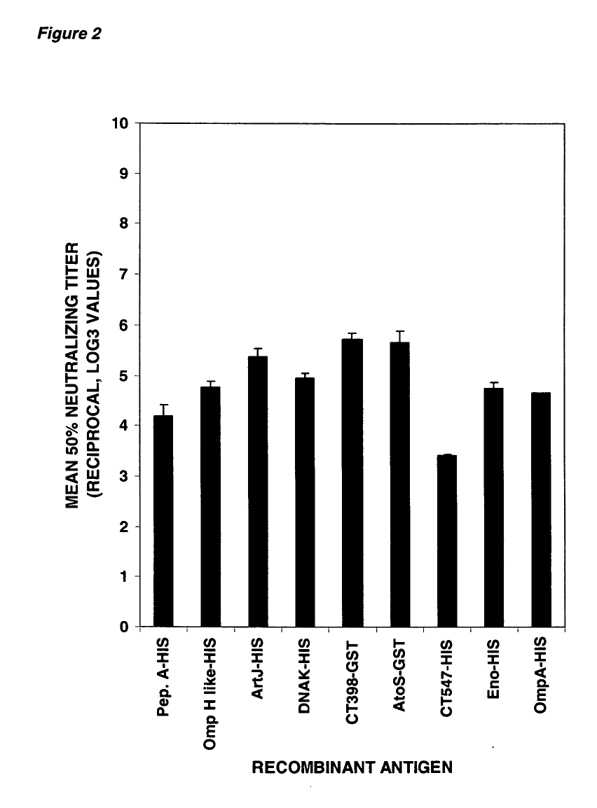

The following columns are set forth in Table 1(a): Gene identification number (Gene ID), Protein ID and the corresponding Current Annotation were retrieved from the D/UW-3/CX genome filed in GenBank (accession number AE001273). Fusion Type: Indicates whether the data was generated from a His or GST fusion peptide (or both). Theoretical Molecular Weight represents the molecular mass (in kilodaltons) which were calculated for predicted mature forms of the referenced protein. Antiserum: Western blot Analysis (WB profile) summarizes the western blot results obtained by probing total EB proteins with antisera against the respective recombinant CT proteins. The number in brackets refers to panel number in FIG. 2. WB results are classified as follows: C indicates Consistent (i.e., the predominant band observed is consistent with the expected molecular weight; additional minor bands may also be present); PC indicates Partially Consistent (i.e., a band of expected molecular weight is present together with additional bands of higher molecular weight or greater intensity); NC represents Nonconsistent (i.e., the detected bands do not correspond to the expected molecular weight); N represents Negative (i.e., no profile obtained). Antiserum: FACS Assay (KS score) includes the results of FACS analysis, expressed as K-S scores. The serum titers giving 50% neutralization of infectivity for the 9 C. trachomatis recombinant antigens described in the text (PepA, ArtJ, DnaK, CT398, CT547, Enolase, MOMP, OmpH-like, Atos). Each titer was assessed in 3 separate experiments (SEM values shown). Antiserum: Neutralizing Titre (reciprocal) represents neutralizing antibody titers for the respective CT antigens. The results are as follows: PepA (CT045) 1:100; ArtJ (CT381) 1:370; DnaK (CT396) 1:230; Hypothetical (CT398) 1:540; Hypothetical (CT547) 1:40; Enolase (CT587) 1:180; MOMP (CT681) 1:160; OmpH-like (CT242) 1:190; AtoS (CT467) 1:500. All of the proteins that showed a K-S score higher than 8.0 have been listed as FACS-positive. Antigen: Reported 2DE/MALDI-TOF detection are depicted as yes/no/? (=not determined) results in the last column of the Table.

TABLE 1(a)
Characterisation of Chlamydia trachomatis (CT) expressed proteins

| | | | | | | Antiserum: | Antiserum: |
|---|---|---|---|---|---|---|---|
| | | | | Theoretical | Antiserum: | FACS | Neutralizing |
| Gene | | | | MWt | WB | assay (K | titre |
| ID | Protein ID | Current annotation | Fusion type | (kDa) | analysis | S score) | (reciprocal) |
| CT045 | PepA | pep A (Leucyl Aminopeptidase A) | HIS | 54.0 | C | 16.81 | 100 |
| CT381 | ArtJ | art3 (Arginine Binding Protein) | HIS | 26.0 | C | 32.54 | 370 |
| CT396 | DnaK | dnaK (HSP-70 heat shock protein) | HIS | 70.6 | C | 34.50 | 230 |
| CT398 | CT398 | Hypothetical protein | His&GST | 29.4 | C | 31.24 | 540 |
| CT547 | CT547 | Hypothetical protein | HIS | 32.6 | PC | 28.21 | 40 |
| CT587 | Enolase | eno (Enolase) | HIS | 45.3 | c | 20.85 | 180 |
| CT681 | MOMP | ompA (Major Outer Membrane Protein) | HIS | 40.1 | c | 34.66 | 160 |
| CT242 | OmpH | ompH-Like Outer Membrane Protein) | HIS | 15.8 | c | <8 | 190 |
| CT467 | AtoS | atoS (2-component sensor histidine kinase) | GST | 39.8 | N | <8 | 500 |
| CT043 | CT043 | hypothetical <<Cpn0387 | GST | 18.4 | ND | 27.53 | |
| CT050 | CT050 | Hypothetical protein | GST | 56.6 | C (1) | 20.68 | |
| CT082 | CT082 | Hypothetical protein | GST | 59.4 | C (2) | 25.63 | |
| CT089 | LcrE | lcrE (Low Calcium response E) | HIS | 43.0 | C (3) | 12.59 | |
| CT128 | Adk | adk (adenylate kinase) | GST | 27.6 | C (4) | 16.00 | |
| CT153 | CT153 | hypothetical >Cpn0176 (6445) | GST | 90.8 | ND | 13.33 | |
| | | <<MAC/perforin domain | | | | | |
| CT157 | CT157 | Phospholipase D Superfamily | GST | 45.2 | C (5) | 19.77 | |
| CT165 | CT165 | Hypothetical protein | GST | 16.8 | C (6) | 10.46 | |
| CT262 | CT262 | hypothetical > Cpn0411 | His-ib | 28.7 | ND | 19.31 | |
| CT266 | CT266 | Hypothetical protein >Cpn0415 (6696) | HIS | 43.9 | PC (7) | 21.29 | |
| CT276 | CT276 | hypothetical (acidic) > Cpn0425 (6706) | GST | 213 | ND | 19.85 | |
| CT296 | dcrA | hypothetical divalent cation dependent | CST | 17.9 | ND | 17.70 | |
| | | regulator (Raulston) | | | | | |
| CT316 | L7/LI2 | rI7 (Ribosomal protein L7/L12) | HIS | 13.4 | C (8) | 9.68 | |
| CT372 | CT372 | hypothetical (basic) | His | 49.3 | ND | 24.77 | |
| CT443 | OmcB | omcB (60 kDa Cysteine-Rich OMP) | HIS | 56.2 | C (9) | 21.28 | |
| CT444 | OmcA | omcA (9 kDa Cysteine-Rich OMP) | GST | 9.0 | PC (10) | 15.00 | |
| CT456 | CT456 | Hypothetical protein | GST | 97.6 | N (11) | 10.90 | |
| CT480 | oppA | oligopeptide binding protein (1 of 5 genes) | pHis&pGST | 58.8 | ND | 27.45/9.48 | |
| CT541 | Mip-like | mip (FKBP-type cis-trans isomerase) | GST | 24.5 | C (12) | 9.94 | |
| CT548 | CT548 | hypothetical | GST | ND | ND | 14.78 | |
| CT559 | YscJ | yscJ (Yop proteins translocation lipoprotein | HIS | 33.3 | C (13) | 23.21 | |
| | | J) | | | | | |
| CT600 | Pal | pal (Peptidoglycan-Associated Lipoprotein) | HIS | 19.1 | C (14) | 10.46 | |
| CT623 | CT623 | CHLPN 76 kDa Homolog | GST | 45.6 | C (15) | 15.89 | |
| CT635 | CT635 | hypothetical | His&GST | ND | ND | 11.62/11.52 | |
| CT671 | CT671 | hypothetical | his | ND | ND | 9.29 | |
| CT713 | PorB | porB (Outer Membrane Protein Analog) | HIS | 34.4 | C (16) | 25.82 | |
| CT823 | HtrA | htrA (DO serine protease) | HIS | 51.4 | PC (17) | 26.62 | |
| CT859 | CT859 | metalloprotease | his&GST | ND | ND | 10.91/9.46 | |
| CT412 | pmpA | OM protein A | His | 105.6 | ND | 10.92 | |
| CT414 | PmpC | pmpC (Putative outer membrane protein C) | GST | 184.9 | C (18) | 9.03 | |
| CT812 | PmpD | pmpD (Putative Outer Membrane Protein D) | GST | 157.6 | N (19) | 10.43 | |
| CT869 | PmpE | pmpE (Putative Outer Membrane Protein E) | HIS | 102.7 | N (20) | 15.28 | |

Similar columns are represented in Table 1(b). In this table, the In-vitro Neutralizing Activity column, indicates either neg (negative) or ND (not determined).

TABLE 1(b)
Characterization of Expressed Chlamydia trachomatis (CT) Proteins cont

| | | | Molecular | K—S |
|---|---|---|---|---|
| Gene ID | Gene Annotation | Fusion Type | Mass (kDa) | Score |
| CT016 | Hypothetical | HIS | 26.63 | 17.94 |
| CT017 | Hypothetical | HIS | 47.79 | 12.18 |
| CT043 | Hypothetical | HIS | 18.38 | 27.53 |
| CT082 | Hypothetical | HIS | 59 | 15.89 |
| CT548 | Hypothetical | GST | 21.9 | 14.78 |
| CT153 | Hypothetical | GST | 90.86 | 13.33 |
| CT262 | Hypothetical | HIS | 28.81 | 19.31 |
| CT276 | Hypothetical | GST | 21.37 | 19.85 |
| CT296 | Hypothetical | GST | 17.98 | 17.70 |
| CT372 | Hypothetical | HIS | 49.00 | 24.77 |
| CT398 | Hypothetical | GST | | 27.03 |
| CT398 | Hypothetical | HIS | | 22.96 |
| CT548 | Hypothetical | GST | | 14.78 |
| CT043 | Hypothetical | HIS | | 27.53 |
| CT635 | Hypothetical | GST | 16.77 | 11.52 |
| CT635 | Hypothetical | HIS | 16.77 | 11.62 |
| CT671 | Hypothetical | HIS | 31 | 20.91 |
| CT671 | Hypothetical | GST | 31 | 18.07 |
| CT089 | Low Calcium | GST | 44 | 11.9 |
| | Response Element | | | |
| | (LcrE) | | | |
| CT812 | PmpD | GST | 168 | 23.48 |
| CT412 | Putative Outer | HIS | 107 | 10.92 |
| | Membrane Protein A | | | |
| CT480 | Oligopeptide | GST | 79.89 | 9.48 |
| | Binding | | | |
| | Lipoprotein | | | |
| CT480 | Oligopeptide | HIS | 79.89 | 27.45 |
| | Binding | | | |
| | Lipoprotein | | | |
| CT859 | Metalloprotease | GST | 34.21 | 9.46 |
| CT859 | Metalloprotease | HIS | 34.21 | 10.91 |
| CT869 | PmpE | GST | 106 | 30.67 |
| CT053 | | | | |

TABLE 1(c)
Characterization of Chlamydia trachomatis Proteins cont

| | | | | | Antiserum: | | | Antiserum: |
|---|---|---|---|---|---|---|---|---|
| | | Identity to | | Theoretical | ELISA titre | Antiserum: | Antiserum: Dot [symbol] | FACS assay |
| Gene ID | Protein ID | Cpn (%) | Current annotation | MWt (kDa) | (reciprocal) | WB analysis | Blot analysis | (K-S score) |
| CT045 | PepA | 53 | pepA (Leucyl Aminopeptidase A) | 54.0 | 320000 | C | P | 16.81 |
| CT381 | ArtJ | 61 | artJ (Arginine Binding Protein) | 26.0 | 320000 | C | P | 32.54 |
| CT396 | DnaK | 84 | dnaK (HSP-70 heat shock protein) | 70.6 | 160000 | PC | P | 34.50 |
| CT398 | CT398 | 70 | Hypothetical protein | 29.4 | 320000 | PC | P | 31.24 |
| CT587 | Enolase | 73 | eno (Enolase) | 45.3 | 40000 | PC | P | 20.85 |
| CT681 | MOMP | 62 | ompA (Major Outer Membrane | 40.1 | 320000 | PC* | P | 34.66 |
| | | | Protein) | | | | | |
| CT242 | OmpH | 60 | ompH-Like Outer Membrane | 15.8 | 80000 | C | P | <9 |
| | | | Protein | | | | | |
| CT043 | CT043 | 85 | hypothetical <<Cpn0387 | 18.4 | 640000 | C (1) | P | 25.29 |
| CT082 | CT082 | 35 | Hypothetical protein. | 59.4 | 160000 | C (2) | P | 25.63 |
| CT089 | LcrE | 42 | lcrE (Low Calcium response E) | 43.0 | 320000 | C (3) | P | 12.59 |
| CT128 | Adk | 42 | adk (adenylate kinase) | 27.6 | 320000 | C (4) | WP | 16.00 |
| CT153 | CT153 | 48 (393) | hypothetical >Cpn0176 (6445) | 90.8 | 640000 | C (5) | P | 13.33 |
| | | | <<MAC/perforin domain | | | | | |
| CT266 | CT266 | 48 | Hypothetical protein | 43.9 | 25000 | C (6) | P | 21.29 |
| | | | >Cpn0415(6696) | | | | | |
| CT276 | CT276 | 57 | hypothetical (acidic) > Cpn0425 | 21.3 | 640000 | C (7) | P | 19.85 |
| | | | (6706) | | | | | |
| CT316 | L7/L12 | 66 | rI7 (Ribosomal protein L7/L12) | 13.4 | 160000 | C (8) | P | 9.68 |
| CT443 | OmcB | 71 | omcB (60 kDa Cysteine-Rich OMP) | 56.2 | 25000 | PC* (9) | P | 21.28 |
| CT444 | OmcA | 50 | omcA (9 kDa Cysteine-Rich OMP) | 9.0 | 600000 | PC (10) | P | 15.00 |
| CT480 | DppA | 67 | ABC transporter solute binding | 79.8 | 640000 | C (11) | P | 27.45 |
| | | | protein | | | | | |
| CT541 | Mip-like | 62 | mip (FKBP-type cis trans | 24.5 | 100000 | C (12) | P | 43.36 |
| | | | isomerase) | | | | | |
| CT548 | CT548 | 56 | hypothetical | 21.09 | 640000 | C (13) | P | 14.78 |
| CT559 | YscJ | 72 | Yop proteins translocation | 33.3 | 50000 | PC (14) | P | 23.21 |
| | | | lipoprotein J | | | | | |
| CT623 | CT623 | 60 | CHLPN 76 kDa Homolog | 45.6 | 10000 | PC (15) | P | 15.89 |
| CT713 | PorB | 59 | porB (Outer Membrane Protein | 34.4 | 160000 | PC (16) | P | 25.82 |
| | | | Analog) | | | | | |
| CT823 | HtrA | 69 | htrA (DO serine protease) | 51.4 | 320000 | PC (17)* | P | 26.62 |
| CT859 | CT859 | 70 | metalloprotease? - lytB | 34.21 | 640000 | C (18) | P | 10.91 |
| CT414 | PmpC | 45 (420) | pmpC (N-term domain) | 184.9 | 400000 | C (19) | N | 27.25 |
| CT812 | PmpD | 32 | pmpD (N-term domain) | 157.6 | 400000 | N (20) | P | 23.10 |
| CT869 | PmpE | 31 | pmpE | 102.7 | 640000 | N (21) | WP | 30.67 |
| CT871 | PmpG | 31 | pmpG | 107.2 | 320000 | C (22) | P | 43.06 |

[symbol]

indicates data missing or illegible when filed

TABLE 1(d)
Characterization of Chlamydia trachomatis Proteins cont

| | | | Antiserum: | | | | | Antigen: |
|---|---|---|---|---|---|---|---|---|
| | | | ELISA titre | | | | | Reported |
| | | | against | | | Antiserum: | Antiserum: | 2DE/ |
| | | | Recombinant | | | FACS | Neutralizing | MALDI- |
| | | | Antigen | Antiserum: | Antiserum:D [symbol] | assay (K [symbol] | titre | TOF |
| Gene ID | Protein ID | Current annotation | (reciprocal) | WB analysis | Blot analysis | score) | (reciproc [symbol] | detection |
| CT045 | PepA | pepA (Leucyl Aminopeptidase A) | 320000 | C | P | 16.81 | 100 | Yes |
| CT381 | ArtJ | artJ (Arginine Binding Protein) | 320000 | C | P | 32.54 | 370 | No |
| CT396 | DnaK | dnaK (HSP-70 heat shock protein) | 160000 | PC | P | 34.50 | 230 | Yes |
| CT398 | CT398 | Hypothetical protein | 320000 | PC | P | 31.24 | 540 | Yes |
| CT587 | Enolase | eno (Enolase) | 40000 | PC | P | 20.85 | 180 | Yes |
| CT681 | MOMP | ompA (Major Outer Membr [symbol] | 320000 | PC* | P | 34.66 | 160 | Yes |
| | | Protein) | | | | | | |
| CT242 | OmpH | ompH-Like Outer Membra [symbol] | 80000 | C | P | <9 | 190 | Yes |
| | | Protein | | | | | | |
| CT043 | CT043 | hypothetical <<Cpn0387 | 640000 | C (1) | P | 25.29 | NA | No |
| CT082 | CT082 | Hypothetical protein. | 160000 | C (2) | P | 25.63 | NA | Yes |
| CT089 | LcrE | lcrE (Low Calcium response E) | 320000 | C (3) | P | 12.59 | NA | No |
| CT128 | Adk | adk (adenylate kinase) | 320000 | C (4) | WP | 16.00 | NA | No |
| CT153 | CT153 | hypothetical >Cpn0176 (644 | 640000 | C (5) | P | 13.33 | NA | No |
| | | <<MAC/perforin domain | | | | | | |
| CT266 | CT266 | Hypothetical protein | 25000 | C (6) | P | 21.29 | NA | No |
| | | >Cpn0415 (6696) | | | | | | |
| CT276 | CT276 | hypothetical (acidic) > Cpn0 [symbol] | 640000 | C (7) | P | 19.85 | NA | No |
| | | (6706) | | | | | | |
| CT316 | L7/L12 | rI7 (Ribosomal protein L7/L12) | 160000 | C (8) | P | 9.68 | NA | Yes |
| CT443 | OmcB | omcB (60 kDa Cysteine-Rich OMP) | 25000 | PC* (9) | P | 21.28 | NA | Yes |
| CT444 | OmcA | omcA (9 kDa Cysteine-Rich OMP) | 600000 | PC (10) | P | 15.00 | NA | No |
| CT480 | DppA | ABC transporter solute bind [symbol] | 640000 | C (11) | P | 27.45 | NA | No |
| | | protein | | | | | | |
| CT541 | Mip-like | mip (FKBP-type cis-trans | 100000 | C (12) | P | 43.36 | NA | Yes |
| | | isomerase) | | | | | | |
| CT548 | CT548 | hypothetical | 640000 | C (13) | P | 14.78 | NA | No |
| CT559 | YscJ | Yop proteins translocation | 50000 | PC (14) | P | 23.21 | NA | No |
| | | lipoprotein J | | | | | | |
| CT623 | CT623 | CHLPN 76 kDa Homolog | 10000 | PC (15) | P | 15.89 | NA | Yes |
| CT713 | PorB | porB (Outer Membrane Pro [symbol] | 160000 | PC (16) | P | 25.82 | NA | Yes |
| | | Analog) | | | | | | |
| CT823 | HtrA | htrA (DO serine protease) | 320000 | PC (17) * | P | 26.62 | NA | Yes |
| CT859 | CT859 | metalloprotease? - lytB | 640000 | C (18) | P | 10.91 | NA | No |
| CT414 | PmpC | pmpC (N-term domain) | 400000 | C (19) | N | 27.25 | NA | No |
| CT812 | PmpD | pmpD (N-term domain) | 400000 | N (20) | P | 23.10 | NA | Yes |
| CT869 | PmpE | pmpE | 640000 | N (21) | WP | 30.67 | NA | No |
| CT871 | PmpG | pmpG | 320000 | C (22) | P | 43.06 | NA | Yes |

[symbol]

indicates data missing or illegible when filed

### Example 1

### Western Blot, FACS and In Vitro Neutralization Assay and Analysis of CT Antigens, as Shown in Table 1(a)

The Western Blot, FACS and In Vitro Neutralization assays and analysis of Tables 1(a) and 1(b) are further discussed in this Example. Preparation of the materials and details of these assays are set forth below.

Preparation of C. trachomatis EBs and chromosomal DNA: C. trachomatis GO/96, a clinical isolate of C. trachomatis serotype D from a patient with non-gonococcal urethritis at the Sant'Orsola Polyclinic, Bologna, Italy, was grown in LLC-MK2 cell cultures (ATCC CCL-7). EBs were harvested 48 h after infection and purified by gradient centrifugation as described previously (See Schachter, J., and P. B. Wyrick. 1994. Methods Enzymol. 236:377-390). Purified chlamydiae were resuspended in sucrose-phosphate transport buffer and stored at −80° C. until use. When required, prior to storage EB infectivity was heat inactivated by 3 h of incubation at 56° C. Chromosomal DNA was prepared from gradient-purified EBs by lysing the cells overnight at 37° C. with 10 mM Tris-HCl, 150 mM NaCl, 3 mM EDTA, 0.6% SDS, 100 μg of proteinase K/ml, sequential extraction with phenol, phenol-chloroform, and chloroform, alcohol precipitation and resuspension in TE buffer, pH 8.

In silico analyses: All the 894 protein coding genes and the corresponding peptide sequences encoded by the C. trachomatis genome UW-3/Cx (Stephens et al., 1998. Science 282: 754-9) were retrieved from the National Center for Biotechnology Information web site (http://www.ncbi.nlm.nih.gov/). Putative surface exposed proteins were selected primarily on the basis of GenBank annotation and sequence similarity to proteins known to be secreted or surface-associated. Sequences annotated as hypothetical, which typically lack significant homologies to well characterized proteins, were analyzed for the presence of leader peptide and/or transmembrane regions with PSORT algorithm (Gardy et al., Nucleic Acids Res. 2003 Jul. 1; 31(13):3613-7). Following these criteria, a set of 158 peptides were selected for expression and in vitro screening.

Cloning and expression of recombinant proteins: Selected ORFs from the C. trachomatis UW-3/Cx genome (Stephens et al., supra) were cloned into plasmid expression vectors so as to obtain two kinds of recombinant proteins: (i) proteins with a hexa-histidine tag at the C terminus (ct-His), and (ii) proteins fused with both glutathione S-transferase (GST) at their N terminus and a hexa-histidine tag at their C terminus (Gst-ct) as described in (Montigiani, et al., 2002. Infect Immun 70:368-79). Escherichia coli BL21 and BL21(DE3) (Novagen) were the recipient of pET21b-derived recombinant plasmids and pGEX-derived plasmids respectively. PCR primers were designed so as to amplify genes without the signal peptide coding sequence. When a signal peptide or processing site was not clearly predictable, the ORF sequence was cloned in its full-length form. Recombinant clones were grown in Luria-Bertani medium (500 ml) containing 100 ug of ampicillin/ml and grown at 37° C. until an optical density at 600 nm (OD600) of 0.5 was reached. Expression of recombinant proteins was then induced by adding 1 mM isopropyl-D-thiogalactopyranoside (IPTG). Three hours after IPTG induction, cells were collected by centrifugation at 6000×g for 20 min. at 4° C. Before protein purification, aliquots of the cell pellets (corresponding to an OD600 of 0.1) were resuspended in sample loading buffer (60 mMTris-HCl [pH 6.8], 5% [wt/vol] SDS, 10% [vol/vol] glycerol, 0.1% [wt/vol] bromophenol blue, 100 mM dithiothreitol [DTT]), boiled for 5 min, and analyzed bySDS-polyacrylamide gel electrophoresis (SDS-PAGE).

Purification of recombinant proteins. The cell pellets obtained from centrifugation of 500 ml induced recombinant E. coli cultures were suspended with 10 ml B-PER™ (Bacterial-Protein Extraction Reagent, Pierce), 1 mM MgCl2, 100 Kunits units DNAse I (Sigma), and 1 mg/ml lysozime (Sigma). After 30 min at room temperature under gentle shaking the lysate was clarified by centrifugation at 30.000 g for 30 min at 4° C. and the supernatant (soluble proteins) was separated from the pellet (debris, insoluble proteins and inclusion bodies).

Soluble His-tagged proteins were purified by an immobilized metal affinity chromatography (IMAC) using 1 ml mini-columns of Ni-activated Chelating Sepharose Fast Flow (Amersham). After loading the column was washed with 20 mM Imidazole and the remaining proteins were eluted by one step elution using 250 mM Imidazole buffer, 50 mM phosphate, 300 mM NaCl, pH 8.0.

Insoluble His-tagged proteins were purified by suspending the pellet, coming from centrifugation of B-PER lysate, in 50 mM TRIS-HCl, 1 mM TCEP (Tris(2-carboxyethyl)-phosphine hydrochloride, Pierce) and 6M guanidine hydrochloride, pH 8.5, and performing an IMAC in denaturing conditions of the clarified solubilized proteins. Briefly: the resuspended material was centrifuged at 30.000 g for 30 min and the supernatant was loaded on 1 ml minicolumns of Ni-activated Chelating Sepharose Fast Flow (Pharmacia) equilibrated with 50 mM TRIS-HCl, 1 mM TCEP, 6M guanidine hydrochloride, pH 8.5. The column was washed with 50 mM TRIS-HCl buffer, 1 mM TCEP, 6M urea, 20 mM imidazole, pH 8.5. Recombinant proteins were then eluted with the same buffer containing 250 mM imidazole.

The soluble GST-fusion proteins were purified by subjecting the B-PER soluble lysate to glutathione affinity purification using 0.5 ml mini-columns of Glutathione-Sepharose 4B resin (Amersham) equilibrated with 10 ml PBS, pH 7.4. After column washing with equilibrium buffer the proteins were eluted with 50 mM TRIS buffer, 10 mM reduced glutathione, pH 8.0.

Protein concentration was determined using the Bradford method.

In some embodiments, a HIS tagged protein is used whereas in other embodiments a GST tagged protein was used. In other instances, combinations of HIS tagged or GST tagged proteins were used. Preferably the immunogenic compositions comprise one or more HIS tagged proteins.

Eluted protein fractions were analyzed by SDS-Page and purified proteins were stored at −20° C. after addition of 2 mM Dithiothreitol (Sigma) and 40% glycerol.

Preparation of mouse antisera: Groups of four 5- to 6-week-old CD1 female mice (Charles River, Como, Italy) were immunized intraperitoneally at days 1, 15, and 28 with 20 ug of purified recombinant protein in Freund's adjuvant. Pre-immune and immune sera were prepared from blood samples collected on days 0 and 43 respectively and pooled before use. In order to reduce the amount of antibodies possibly elicited by contaminating E. coli antigens, the immune sera were incubated overnight at 4° C. with nitrocellulose strips adsorbed with an E. coli BL21 total protein extract.

Immunological assays: For Western blot analysis, total proteins from purified C. trachomatis GO/96 serotype D EBs (2 ug per lane) were separated by SDS-PAGE and electroblotted onto nitrocellulose membranes. After 30 min. of saturation with PBS-dried skimmed milk (5% w/v) membranes were incubated overnight with preimmune and immune sera (standard dilution 1:400) and then washed 3× with phosphate-buffered saline (PBS)-Tween 20 (0.1% v/v). Following a 1 hour incubation with a peroxidase-conjugated anti-mouse antibody (final dilution 1:5,000 Amersham;) and washing with PBS-Tween, blots were developed using an Opti-4CN Substrate Kit (Bio-Rad).

Flow cytometry assays: Analyses were performed essentially as previously described (See Montigiani et al., supra). Gradient purified, heat-inactivated GO/96 serotype D EBs (2×105 cells) from C. trachomatis resuspended in phosphate-saline buffer (PBS), 0.1% bovine serum albumin (BSA), were incubated for 30 min. at 4° C. with the specific mouse antisera (standard dilution 1:400). After centrifugation and washing with 200 μl of PBS-0.1% BSA, the samples were incubated for 30 minutes at 4° C. with Goat Anti-Mouse IgG, F(ab)′2-specific, conjugated with R-Phycoerythrin (Jackson Immunoresearch Laboratories Inc.). The samples were washed with PBS-0.1% BSA, resuspended in 150 μl of PBS-0.1% BSA and analyzed by Flow Cytometry using a FACSCalibur apparatus (Becton Dickinson, Mountain View, Calif.). Control samples were similarly prepared. Positive control antibodies were: i), a commercial anti-C. pneumoniae specific monoclonal antibody (Argene Biosoft, Varilhes, France) and, ii), a mouse polyclonal serum prepared by immunizing mice with gradient purified C. trachomatis EBs.

Background control sera were obtained from mice immunized with the purified GST or HIS peptide used in the fusion constructs (GST control, HIS control). FACS data were analyzed using the Cell Quest Software (Becton Dickinson, Mountain View, Calif.). The significance of the FACS assay data has been elaborated by calculating the Kolmogorov-Smirnov statistic (K-S score.) (See Young, I. T. 1977. J Histochem Cytochem 25:935-41). The K-S statistic allows determining the significance of the difference between two overlaid histograms representing the FACS profiles of a testing protein antiserum and its relative control. All the proteins that showed a K-S score higher than 8.0 have been listed as FACS positive, being the difference between the two histograms statistically significant (p<0.05). The D/s(n) values (an index of dissimilarity between the two curves) are reported as “K-S score” in Tables 1(a) and 1(b).

In vitro neutralization assays: In vitro neutralization assays were performed on LLC-MK2 (Rhesus monkey kidney) epithelial cell cultures. Serial four-fold dilutions of mouse immune and corresponding preimmune sera were prepared in sucrose-phosphate-glutamic acid buffer (SPG). Mouse polyclonal sera to whole EBs were used as positive control of neutralization, whereas SPG buffer alone was used as negative control of neutralization (control of infection). Purified infectious EBs from C. trachomatis GO/96 serotype D were diluted in SPG buffer to contain 3×10⁵ IFU/ml, and 10 ul of EBs suspension were added to each serum dilution in a final volume of 100 μl. Antibody-EB interaction was allowed to proceed for 30 min at 37° C. on a slowly rocking platform. The 100 ul of reaction mix from each sample was used to inoculate PBS-washed LLC-MK2 confluent monolayers (in triplicate for each serum dilution), in a 96-well tissue culture plate, and centrifuged at 805×g for 1 hour at 37° C. After centrifugation Eagle's minimal essential medium containing Earle's salts, 20% fetal bovine serum and 1 ug/ml cycloheximide was added. Infected cultures were incubated at 37° C. in 5% CO₂ for 72 hours. The monolayers were fixed with methanol and the chlamydial inclusions were detected by staining with a mouse anti-Chlamydia fluorescein-conjugated monoclonal antibody (Merifluor Chlamydia, Meridian Diagnostics, Inc.) and quantified by counting 5 fields per well at a magnification of 40×. The inhibition of infectivity due to EBs interaction with the immune sera was calculated as percentage reduction in mean IFU number as compared to the SPG (buffer only)/EBs control. In this calculation the IFU counts obtained with immune sera were corrected for background inhibition of infection due to the corresponding pre-immune mouse serum. According to common practice, the sera were considered as “neutralizing” if they could cause a 50% or greater reduction in infectivity. The corresponding neutralizing titer was defined as the serum dilution at which a 50% reduction of infectivity was observed. Experimental variability was evaluated by calculating the standard error of measurement (SEM), from three titration experiments for each recombinant antigen, as shown in FIG. 2.

Preparation of Dot Blots

A dot-blot assay to assess surface reactivity of the antisera, was performed essentially as described by Zhang et al ((1987) J Immunol 138(2) 575-81) and Kawa and Stephens ((2002) J Immunol 168(10) 5184-91). The dot blot results provided in Tables 1(c) and 1(d) are listed as either Positive (P) or Weakly Positive (WP). A nitrocellulose membrane pre-soaked in PBS was assembled in a dot-blot apparatus (Bio-Rad) and a 30 ul suspension of CT EBs (equivalent to 5 ug of protein) in PBS was added to the wells. A light vacuum was applied to remove all liquid. The membrane was removed and treated with 2% dried milk in PBS for 1 hr at room temperature and washed three times in PBS-Tween. After reassembling the filtration apparatus, the antisera (50 fold dilution) were added to the wells and incubated for 1 hr at room temperature. After three washings with 200 ul of PBS-Tween, the membrane was removed and again washed. A 1/5000 dilution of goat anti-mouse peroxidase-conjugated antibody (Amersham Biosciences) was added as a secondary antibody for 1 hr at room temperature. After three washes in PBS-Tween, the presence of the secondary antibody on the membrane was detected using the Opti-4CN Substrate Kit (Biorad). Positive control antibodies were an antiserum raised against whole Cpn EBs (200-fold dilution) and an antiserum (50-fold dilution) to the OmcB (Omp2) outer membrane protein. Negative controls included pre-immune mouse sera and a polyclonal antiserum to the GST recombinant-fusion moiety alone.

Results of the Western Blot, FACS, Dot Blot and In Vitro Neutralization assays and analysis are depicted in Tables 1(a)-(d) are further discussed below.

In silico selection: The genomic ORFs to be expressed and submitted to functional screenings were selected on the basis of in silico analyses and literature searches, using bioinformatics tools and criteria similar to those described in a previous similar study on C. pneumoniae (Montigiani, et al., 2002). Essentially, we searched the genome of C. trachomatis serovar D for ORF's encoding proteins likely to be located on the surface of EBs. In order to maximize the chances of identifying bacterial surface proteins we initially selected C. trachomatis proteins having a significant sequence similarity to proteins found to be surface exposed in C. pneumoniae as previously reported (Montigiani, et al., 2002). A second step search was based essentially on the presence of a recognizable leader peptide (mostly as detected by the PSORT software), predicted transmembrane regions, and/or remote sequence similarities to surface proteins of other gram-negative bacteria detected with PSI-Blast runs against the non-redundant GenBank protein database. A third criterion was the addition to the panel of proteins described as immunogenic in animal models and humans. Using this procedure we selected a total of 158 ORFs, 114 of which had at least 40% of identity to proteins of C. pneumoniae, while 44 remained below such threshold and were considered as C. trachomatis specific.

Antigen cloning and expression: The 158 ORFs were amplified by PCRs and cloned in two different E. coli expression vectors in order to obtain each antigen as GST and/or His-tag fusion protein. Considering that the presence of an N-terminal signal peptide could have induced a possible targeting of the recombinant protein toward the E. coli cytoplasmic membrane, the N-terminal signal peptide nucleotide sequence was excluded from the expression construct. By the analysis of the ORFs expression we found that 94% of the selected genes could be expressed and 87% of them (corresponding to 137 different ORFs) could also be purified to recombinant fusion proteins that could be used as antigens for mice immunization. In total, 259 recombinant C. trachomatis fusion proteins, deriving from the 137 different genes cloned, were obtained and analyzed for their quality in order to be used as antigens for mice immunization. Mice were immunized with 201 recombinant C. trachomatis fusion proteins to produce mouse sera that have been analyzed for their capability to recognize surface exposed proteins on C. trachomatis EBs and their capability of interfering with the process of in vitro infection of epithelial cell culture.

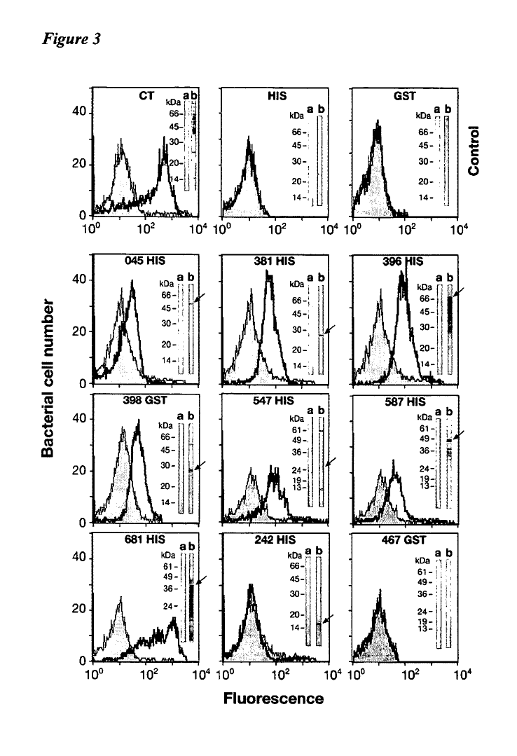

Identification of surface exposed proteins by flow cytometry: Mice were immunized with 201 recombinant C. trachomatis fusion proteins to produce mouse sera that have been analyzed both for their capability to recognize surface exposed proteins on C. trachomatis EBs and their capability of interfering with the process of in vitro infection of epithelial cell culture. Immunofluorescent staining of C. trachomatis EBs and flow cytometric analysis have been used to investigate the capability of mouse sera, obtained by immunization with a panel of 137 different C. trachomatis recombinant antigens, to recognize possibly surface exposed proteins. We had previously shown that flow cytometry can be a very useful tool to detect antibody binding to the surface of chlamydial EBs, by identifying a new panel of C. pneumoniae surface exposed proteins. Although C. trachomatis serovar L and E had already been analyzed by flow cytometry (See Waldman, et al., (1987) Cytometry 8, 55-59; and Taraktchoglou, et al., (2001). Infect Immun 69, 968-76), we first verified if this method could also be applied to C. trachomatis serovar D EBs analysis, by setting up a series of positive and negative controls. As shown in FIG. 3, Panel A, a mouse polyclonal serum obtained by immunizing mice with purified whole C. trachomatis serovar D EBs, can significantly shift the flow cytometric profile of the bacterial cell population, as compared to a negative, pre-immune serum. As a positive control we also used a commercial anti-MOMP C. trachomatis specific monoclonal antibody (Argene), which gave a similar result as the polyclonal serum (data not shown). We also set up a series of negative controls, to exclude possible cross-reactions between mouse sera and the chlamydial cell surface. In particular sera obtained by immunizing mice with the protein fraction eluted from the Ni columns loaded with a BL21(pET21b+) protein extract (His control, FIG. 3, Panel 2) and with GST protein (GST control, FIG. 3, Panel 3) were compared to the respective pre-immune sera. Negative controls never showed a shift of the histogram as compared to pre-immune sera. The control results indicated the specificity and reliability of the flow cytometric assay we set up.

We then analyzed all sera raised against recombinant C. trachomatis antigens for their capability to recognize surface exposed proteins on purified EBs, as determined by FACS binding assay. All the proteins that showed a K-S score higher than 8.0 have been listed as FACS positive, being the difference between the testing and the control histograms statistically significant (p<0.05). Of 137 different gene products analyzed, 28 showed to be able to induce antibodies capable of binding to the surface of purified EBs. Proteins that showed a positive result have been listed in Tables 1(a) and 1(d). The protein list in Table 1(a) is divided into two sections: (i) proteins that gave a positive result in the FACS assay and/or in the neutralization assay, therefore considered to be possibly surface exposed and with a neutralizing effect; (ii) proteins that showed to be able to induce antibodies directed versus surface exposed proteins of the EBs but did not show a detectable neutralizing effect. A comparative analysis of the proteins that resulted to be surface exposed in the C. trachomatis genomic screening shows that 21 out of 28 FACS positive antigens have a degree of homology higher than 40% to C. pneumoniae proteins that, as published in our previous work (Montigiani, et al., 2002), are likely surface exposed.

Analysis of the antisera to the recombinant antigens by Western blotting: The panel of sera was also screened by Western blot analysis on whole protein extracts of purified chlamydial EBs, in order to visualize their capability to recognize a band of the expected molecular weight. The results of this analysis are reported in Tables 1(a) and 1(d), while the Western blot profiles are shown in FIG. 1. In total, 22 out of the 30 sera described in Table 1(a) resulted to be “consistent”, that is they appeared to recognize a band of the expected molecular weight on EBs protein extracts. Four sera, (anti-CT547, anti-CT266, anti-CT444, anti-CT823) were classified as “partially consistent”, due to the presence of a band at the expected molecular mass plus few different bands of weaker intensity. Finally, four sera gave a negative Western blot pattern (anti-CT467, anti-CT456, anti-CT812, anti-CT823). Three out of the four Western blot negative sera (anti-CT456, anti-CT812, anti-CT823) gave a positive result in the FACS binding assay, even if with not very high K-S scores (K-S<15). It is worth noting that two of the Western blot negative sera were raised against antigens (CT812, CT823) belonging to the Pmp family (PmpD and PmpG), a Chlamydia specific family of complex proteins many of which have already been localized on the chlamydial cell surface at least in C. pneumoniae (See, e.g., Knudsen et al., (1999) Infect Immun 67, 375-83; Christiansen et al., (1999) Am Heart J 138, S491-5; Mygind, et al., (2000) FEMS Microbiol Lett 186, 163-9; and Vandahl, et al., (2002) BMC Microbiol 2, 36). The Western blot negative serum obtained by immunization with CT467 (AtoS) was scored as negative also in the FACS assay, but surprisingly it showed a high neutralizing titer (FIG. 2).

Evaluation of the antisera for in vitro neutralizing properties: An in vitro neutralization assay on purified C. trachomatis EBs allowed us to identify neutralizing antigens. Infectious EBs were pre-incubated with the mouse antisera obtained with C. trachomatis recombinant antigens and then tested for their capability to infect a monolayer of epithelial cells. By using this assay, as summarized in Table 1 (a) (section 1) 9 sera have proved to be effectively neutralizing at a dilution higher than 1:30. These 9 sera were obtained by immunizing mice with recombinant proteins encoded by the following C. trachomatis genes: pepA (CT045), encoding a leucyl aminopeptidase; artJ (CT381), encoding a putative extracellular solute (possibly Arginine) binding protein of an aminoacid transport system; dnaK (CT396), encoding a well described chaperonin of the hsp70 family; two “hypothetical” genes CT398 and CT547; eno (CT587), encoding a protein homologous to bacterial enolases, glycolytic enzymes that can be found also on bacterial surfaces; ompA (CT681), encoding the major outer membrane protein; CT242 (OmpH-like), encoding a protein homologue to of the OmpH family of bacterial proteins, some members of which have been reported to be chaperones involved in outer membrane byosinthesis; atoS (CT467), encoding a putative sensor member of a transport system. As shown in FIG. 2, and summarized in Table 1(a), three of the recombinant antigens (ArtJ (CT381), CT398 and AtoS (CT467)) were able to induce antibodies with high neutralizing activity (neutralizing serum titers above 1:300); four of them (DnaK (CT396), Enolase (CT587), OmpA (and OmpH-like (CT242)) induced sera with intermediate neutralizing titers (between 1:180 and 1:300), finally sera raised against two proteins (PepA (CT045) and CT547) had titers equal or less than 100. FIG. 3, on Panels 4 to 12, shows the FACS profiles of the 9 proteins that resulted to be neutralizing, demonstrating that 7 of them are able to induce antibodies directed versus the surface of EBs, while two of them (OmpH-like and AtoS) did not show this capability. The Western blot profiles, against whole-EBs protein extracts, of the sera raised against the FACS-positive neutralizing antigens (FIG. 3) resulted to be either fully consistent, i.e. with a single band of the expected molecular weight (CT045-PepA, CT381-ArtJ) or partially consistent, i.e. showing a major band of the expected molecular weight besides other bands (CT396-DnaK, CT398, CT547, CT587-Enolase, CT681-MOMP). However, in the case of CT396 (DnaK) and CT681 (MOMP), it should be noted that previous work using 2D electrophoretic mapping and either immunoblotting with a specific monoclonal (Bini, et al., (1996) Electrophoresis 17, 185-90) or spot identification by mass spectrometry (Shaw, et al., (2002) Proteomics 2, 164-86) shows that these proteins do appear in EB extracts as multiple electrophoretic species of different Mw, probably due to processing and/or post-translational modifications. Of the 3 remaining ‘partially consistent’ profiles, those obtained with the antisera to recombinant CT398 and CT547-Enolase show that the antibodies recognize predominantly a band of the expected size, whereas in the case of the hypothetical CT547 there is in fact a doubt about the specificity of the antiserum. The two FACS negative and neutralizing antigens showed a different behavior. While the Western blot profile of CT242 (OmpH-like) is fully consistent showing a single band of the expected molecular weight (FIG. 3, Panel 8), the blot of CT467 (AtoS) resulted to be completely negative (FIG. 3, Panel 9).

In the case of the anti-OmpH (CT242) serum, the apparent contradiction between FACS and Western blot profiles could be explained assuming a different sensitivity between the two assays. A positive result was obtained with CT242 using the dot-blot assay suggesting that CT242 is immunoaccessible on the Chlamydial surface. However, the AtoS (CT467) results remain contradictory. Considering that the above findings could be partially explained by the fact that for safety reasons the FACS analyses were performed on heath-inactivated preparations of EB and that the inactivation procedure could have totally (anti-AtoS) or partially (anti-OmpH) destroyed conformational epitopes essential for antibody binding, we also tested these antisera in a dot-blot assay (REF) using infectious EBs spotted on a nitrocellulose membrane, as described by Kawa and Stephens (Kawa and Stephens, 2002). However, the dot-blot assay results for AtoS (CT467) only confirmed the results obtained with the FACS assay.

Further discussion and analysis of the results of the Western Blot, FACS and In Vitro Neutralization assays and analysis as shown in Tables 1(a)-(d) follows below.

Tables 1(a)-(d) present the results of FACS and the ‘in vitro neutralization’ assays obtained from sera raised against a set of C. trachomatis recombinant fusion proteins, of which, so far, 9 “neutralizing” antigens were identified. With the exception of MOMP, none of these antigens has been previously reported as neutralizing. Previous literature also describes PorB (CT713) as a second neutralizing protein (See Kawa, D. E. and Stephens, R. S. (2002)). Antigenic topology of chlamydial PorB protein and identification of targets for immune neutralization of infectivity. (J Immunol 168, 5184-91). However, as shown in Table 1(a), the serum against our recombinant form of PorB failed to neutralize Chlamydia infection in vitro. This discrepancy may be explained considering that our recombinant antigen was water-insoluble and therefore it might have lost the correct conformation required to induce neutralizing antibodies. The possibility of a similar situation should be kept in mind also in the interpretation of data relative to the other ‘insoluble’ antigens. It is interesting to note that, besides MOMP, other proteins in this selection, including PepA, DnaK, HtrA and PorB, have been reported as proteins which are immunogenic in the course of genital tract infection in humans.

### Example 2

Western Blot, FACS and In Vitro Neutralization Assay and Analysis of CT Antigens, as Shown in Table 1(b)

Table 1(b) also provides the FACS results obtained from sera raised against a set of 17 Chlamydia trachomatis recombinant fusion proteins, these being: CT016, CT017, CT043, CT082, CT153, CT262, CT276, CT296, CT372, CT398, CT548, CT043, CT635, CT671 (all Hypothetical Proteins). CT412 (Putative Outer Membrane Protein), CT 480 (Oligopeptide Binding Protein), CT859 (Metalloprotease), CT089 (Low Calcium Response Element—LcrE), CT812 (PmpD) and CT869 (PmpE). FACS analysis was carried out on either the HIS fusion and/or the GST fusion. All of these CT recombinant fusion proteins showed a K-S score higher than 8.0 and were deemed FACS positive. With the exception of CT398, CT372 and CT548 at least none of these Hypothetical proteins has been previously reported as FACS positive. In addition, the following proteins: CT050 (Hypothetical), CT165 (Hypothetical), CT711 (Hypothetical) and CT552 (Hypothetical) also showed a K-S score higher than 8.0 and were deemed FACS positive. None of these four proteins has been previously reported as FACS positive. All of these Hypothetical CT antigens are generally regarded are CT specific antigens and do not have a C. pneumoniae counterpart.

### Example 3

### Immunizations with Combinations of the First Antigen Group

The following example illustrates immunization with various combinations of CT antigens. Mixtures of 5 CT antigens were prepared as described above. The antigens are expressed and purified. Compositions of antigen combinations are then prepared comprising five antigens per composition (and containing 15 μg of each antigen per composition).

Immunization Schedule

| | | Route of |
|---|---|---|
| Group | Immunizing Composition | Delivery |
| 1 | Mixture of 5 antigens | Intra-peritoneal or |
| | (15 μg/each) + CFA | intra-nasal |
| 2 | Mixture of 5 antigens | Intra-peritoneal or |
| | (15 μg/each) + AlOH (200 μg) | intra-nasal |
| 3 | Mixture of 5 antigens | Intra-peritoneal or |
| | (15 μg/each) + CpG (10 ug) | intra-nasal |
| 4 | Mixture of 5 antigens | Intra-peritoneal or |
| | (15 μg/each) + AlOH (200 μg) + CpG | intra-nasal |
| | (10 μg) | |
| 5 | Complete Freunds Adjuvant (CFA) | Intra-peritoneal or |
| | | intra-nasal |
| 6 | Mixture of 5 antigens | Intra-peritoneal or |
| | (5 μg/each) + LTK63 (5 μg) | Intranasal |
| 7 | AlOH (200 μg) + CpG (10 μg) | Intra-peritoneal or |
| | | intra-nasal |
| 8 | CpG (10 μg) | Intra-peritoneal or |
| | | intra-nasal |
| 9 | LTK63 (5 μg) | Intra-peritoneal or |
| | | intra-nasal |

Mice are immunized at two week intervals. Two weeks after the last immunization, all mice are challenged by intravaginal infection with Chlamydia trachomatis serovar D. When mucosal immunization (e.g. intra-nasal(in)) is used, the animal model is also challenged mucosally to test the protective effect of the mucosal immunogen.

Test Challenges: The mice were challenged intravaginally with 10⁵ IFU of purified EBs (Serovar D), 2 weeks after the last immunization dose. A read out of vaginal swabs every 7 days up to 28 days after challenge. The following assays were also carried out on pre-challenge sera: Serological analysis: FACS, WB, Neutralization assay and ELISA. The ELISA were performed by coating plates with each recombinant antigen and testing the reaction of pre-challenge immune sera from single mice immunized with the combination of five CT antigens. The data is expressed as the mean value calculated for each group expressed as mean ELISA units. The Chlamydia specific antibody type (IgG, IgA etc) and isotype was checked in serum post immunization but pre-challenge. The purpose of the serum studies was to determine how the mice responded to immunization with the CT antigen combinations. The purpose of the vaginal washes was to determine how the mice responded to the Chlamydia bacterial challenge. Chlamydia specific antibody analyses in terms of antibody type (IgG and IgA) and antibody subtype were also carried out on the vaginal washes.

Negative Controls: The negative control used was the immunoregulatory agent alone (e.g. CFA or AlOH and/or CpG).

Positive “live” EB controls: The positive control used was an extract from live Chlamydia Elementary Bodies (EBs). Here the mice were infected with live Chlamydia EB at the same time that the test CT combination antigens are being administered. The “live” EB positive control animals were infected for about 1.5 months (i.e. 6 weeks) (because 3 doses of CT antigenic combinations were administered every 2 weeks (i.e. over a total of 6 weeks). The animals (mice) infected with “live” EB developed a natural immunity and resolved the infection (because Chlamydia infection in mice is a transient infection). When the mice were vaccinated with the CT antigenic combinations were then challenged with “live” EB, the positive control “live” EB mice were also re-challenged (i.e. they were given a second dose of “live” EB). As the “live” EB positive control group developed a natural immunity, they cleared the second re-challenge quickly.

Infection Control: In this group, the mice were only challenged with “live” EB at the same time that the “Positive Live EB controls were re-challenged and the test CT group was challenged. The purpose of this control group was to check for a possible protective effect from the negative control group (i.e. the group immunized with immunoregulatory agent alone)

### OVERALL DISCUSSION

According to a genomic strategy aiming at the identification of new vaccine candidates, which gave promising results for other bacterial pathogens, we expressed in E. coli, as recombinant fusion proteins, 158 ORFs selected in silico from the C. trachomatis genome, and likely to encode peripherally located proteins. Polyclonal antibodies to these proteins were raised in mice and assessed, in parallel screenings, (i), for their capacity to bind purified Chlamydiae in a flow cytometry assay (identifying FACS-positive sera and corresponding antigens), and, (ii), for their capacity to induce a >50% inhibition of Chlamydial infectivity for in vitro cell cultures (neutralizing sera and antigens). The specificity of the antisera, which were partially purified by adsorption on E. coli protein extracts, was assessed by Western Blot analysis of the sera diluted 1:400 (the same dilution found optimal for the FACS assay screening) which were tested against protein extracts of gradient-purified elementary bodies of C. trachomatis. The Western Blot results showed that the majority of the 30 FACS positive and/or neutralizing antisera recognized either a single protein band of expected molecular size, or that a band consistent with the expected chlamydial antigen was anyway predominant in the WB profile, with only minor bands of different size. In fact only for 5 antigens a doubt remained as to the true specificity of the antiserum, namely in the case of the CT547 protein, for which the expected band was present but not predominant, and the 4 cases for which the WB obtained was completely blank (CT456, CT476-AtoS, and the two fusion proteins for pmpD (CT812) and pmpE (CT869).

The parallel screenings identified FACS-positive sera and corresponding antigens, and, so far, 9 ‘neutralizing’ antisera and antigens (Table 1(a)). Seven of these (the recombinant forms of PepA (CT045), ArtJ (CT381), DnaK (CT396), Enolase (CT587); the 2 hypothetical products of CT398 and CT547, and the well studied product of ompA better known as the Major Outer Membrane Protein, MOMP (CT681), of C. trachomatis) were both FACS-positive and neutralizing in vitro: the neutralization data therefore seem to confirm that the binding observed in the FACS assay occurred to intact infectious EBs. On the contrary, the two recombinant antigens obtained for the OmpH-like (CT242) and AtoS (CT467) proteins elicited antibodies with in vitro neutralizing properties, but surprisingly failed to show any measurable binding in the FACS assay (FIGS. 2 and 3). The results obtained for CT242 and CT467 are surprising and unexpected as these antigens appear not to be surface-exposed and yet both have high in-vitro neutralizing titers. CT242, however, did have a positive result in the dot-blot assay suggesting that it is immunoaccessible on the Chlamydial surface.

AtoS (CT467): AtoS is a particular case in that the antiserum failed to detect any protein species by Western Blot analysis, and gave negative FACS assay results (with a K-S score below cut-off threshold). Nevertheless this antiserum yielded one of the best neutralization titers, second only to that elicited by the CT398 ‘hypothetical’ protein. Interestingly, in the previous similar screening on Chlamydia pneumoniae (Cpn) antigens (Montigiani et al (2002) Infect Immun 70: 368-379), the antiserum to the homolog Cpn-AtoS proved again to be WB negative, but in this case FACS positive (KS=14.61) and capable of neutralizing (average titer=270) Cpn in vitro infection of the same cell line used in the present study. The apparent inconsistency of these results may be explained by considering that an antigen present in very small amounts in the EB sample could bind too little antibody to be detected in the FACS binding assay, however it could become detectable by the in vitro neutralization assay owing to the possibility of using higher concentration of antibodies and to the amplification provided by the chlamydial replication in this type of assay. The hypothesis that AtoS is somehow lost in purified EBs, e.g. due to a particular instability, is in agreement with the fact that the AtoS protein, shown to be the sensor moiety of a 2-component system composed by AtoS and AtoC was never observed so far by mass spectrometry analysis of 2DE proteomic map nor in any of 3 CT serotypes whereas the expression of the presumably equally abundant AtoC subunit was detected in the 2DE map of serotype-A CT by MALDI-TOF analysis.

CT082 (Hypothetical Protein): CT082 (Hypothetical Protein) is part of an operon annotated as a late transcription unit, and the expression of this ORF has been detected in the EB proteome. It is interesting that our data now indicate the likely exposure of the CT082 protein on the EB surface, supported by a relatively high K-S score (25.62) in the FACS assay. This localization together with its late expression in the replicative cycle suggests an important role of CT082 for some of the multiple EB functions. Surprisingly, we could not detect a sufficient infectivity neutralization mediated by our anti CT082 antiserum. However, as pointed out above, a negative results in a screening study is not to be taken as definitive because many factors (type of recombinant expression, quality of antibody response, the necessarily artificial conditions of the in vitro neutralization assay) may influence the outcome and affect the sensitivity of these assays.

CT398 (Hypothetical Protein): The CT398 antiserum yielded the best neutralization titre in this study. The biological function of this hypothetical protein is unknown. However its presence in the EB proteome has been confirmed by mass spectrometry analysis. Our data now indicate its surface localization and neutralizing properties, and in silico analysis, although an N-terminal signal peptide is not detected by algorithms like PSORT, indicates the presence of a predicted coiled-coil structure between amino-acid residues 11 and 170 which is often present in bacterial surface proteins. Homology searches indicate some homology to a human muscle protein (MYST_HUMAN) and a structural similarly hit with gi|230767|pdb|2TMA|A Chain A, Tropomyosin.

The negative results obtained in these studies are to be considered only negative in relation to the specific procedures and conditions adopted in the screening tests. That is, a negative result may simply be a function of the assay sensitivity. A typical example of such situation is represented by the recombinant porB protein (a conserved dicarboxylate-specific porin which may feed the Chlamydial TCA cycle) which in our hands proved to be surface exposed, in agreement with published data but unable to induce neutralizing antibodies. However, as shown by other workers in the field, porB is in fact also a neutralizing antigen. The discrepancy can be explained considering that the recombinant porB used in these studies. In order to display its neutralizing activity, the initially insoluble recombinant porB had to be refolded by extraction with 1% octlyglucoside and a dialysis step against PBS. Therefore, the neutralizing activity of porB clearly depends on its folding and in our screening work we may have obtained a recombinant porB with a folding which allowed the detection of surface exposure in the FACS assay but lost the neutralizing epitope(s). A similar situation could have been envisaged, from literature data, for the other known porin of Chlamydia, that is for the ompA gene product MOMP (CT681), the best studied vaccine candidate so far, which was also described as possessing folding dependent neutralization properties. Accordingly, one could have expected that in absence of specific refolding steps, our screening results could have failed to detect recombinant MOMP as neutralizing. This however was not the case, and in fact the presence of MOMP within the short list of neutralizing antigens acquires in a way the value of an internal positive control.

The project described herein took advantage from previous work by selecting as a first option a number of C. trachomatis genes considered orthologous (up to 40% identity in the encoded polypeptide) to ‘FACS-positive’ genes of C. pneumoniae, i.e. to genes which when expressed as GST or (6) His fusion proteins elicited antibodies binding to purified C. pneumoniae cells. In Table 1(a) the names of CT proteins which had a corresponding positive screening results in C. pneumoniae are shaded, and it can be noted that 70% of the CT FACS-positive antigens we report have a Cpn ortholog previously described as FACS-positive (See also Tables 1(c) and 1(d)). For general comments on the types of proteins so detected as potential constituents of the chlamydial EB surface, and degree of expected agreement of these experimental finding with the current in silico annotations, we therefore refer the reader to the discussion of the previous results (Montigiani et al (2002) ibid). As far as the neutralization assay is concerned, the published Cpn work did not included this type of assay, however subsequent work from our laboratory identified in the FACS-positive set, at least 10 Cpn neutralizing antigens (Finco et al, (Vaccine 2004) entitled “Identification of new potential vaccine candidates against Chlamydia pneumoniae by multiple screening” available online at www.sciencedirect.com). It is noteworthy that the AtoS, ArtJ, Enolase and OmpH-like antigens (4 of the 9 neutralizing antigens identified in this study) when expressed as Cpn specific allelic variants have neutralizing properties for Cpn in vitro infectivity as well. In contrast with the precedent C. pneumoniae study, when the majority of the Cpn Pmp's yielded soluble and ‘FACS-positive’ fusion proteins, in the present study we obtained only 4 FACS-positive Pmp fusions proteins out of 9 Pmps identified in the CT genome.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be covered by the present invention.

1. An immunogenic composition comprising a combination of Chlamydia trachomatis antigens CT381 and CT456.

2. The immunogenic composition of claim 1 further comprising a TH1 adjuvant and a TH2 adjuvant.

3. The immunogenic composition of claim 2 wherein the TH1 adjuvant elicits an enhanced cell-mediated immune response.

4. The immunogenic composition of claim 2 wherein the TH2 adjuvant elicits an enhanced antibody response.

5. The immunogenic composition of claim 2 wherein the TH1 adjuvant is selected from the group consisting of saponin formulations, virosomes, virus like particles, non-toxic derivatives of enterobacterial lipopolysaccharide (LPS), and immunostimulatory oligonucleotides.

6. The immunogenic composition of claim 2 wherein the TH2 adjuvant is selected from the group consisting of mineral containing compositions, oil-emulsions, ADP-ribosylating toxins, and detoxified derivatives of ADP-ribosylating toxins.

7. The immunogenic composition of claim 2 wherein the TH1 adjuvant is an immunostimulatory oligonucleotide containing a CpG motif.

8. The immunogenic composition of claim 2 wherein the TH2 adjuvant is an aluminum salt.


SEQUENCE LISTING (186 sequences)

SEQ ID NO: 1 - protein, 499 aa, Chlamydia trachomatis
MVLLYSQASWDKRSKADALVLPFWMKNSKAQEAAVVDEDYKLVYQNALSNFSGKKGETAFLFGNDHTKEQKIVLLGLGKSEEVSGTTVLEAYAQATTVLRKAKCKTVNILLPTISQLRFSVEEFLTNLAAGVLSLNYNYPTYHKVDTSLPFLEKVTVMGIVSKVGDKIFRKEESLFEGVYLTRDLVNTNADEVTPEKLAAVAKDLAGEFASLDVKILDRKAILKEKMGLLAAVAKGAAVEPRFIVLDYQGKPKSKDRTVLIGKGVTFDSGGLDLKPGKAMITMKEDMAGAATVLGIFSALASLELPINVTGIIPATENAIGSAAYKMGDVYVGMTGLSVEIGSTDAEGRLILADAISYALKYCNPTRIIDFATLTGAMVVSLGESVAGFFANNDVLARDLAEASSETGEALWRMPLVEKYDQALHSDIADMKNIGSNRAGSITAALFLQRFLEDNPVAWAHLDIAGTAYHEKEELPYPKYATGFGVRCLIHYMEKFLSK

SEQ ID NO: 2 - protein, 421 aa, Chlamydia trachomatis
MTASGGAGGLGSTQTVDVARAQAAAATQDAQEVIGSQEASEASMLKGCEDLINPAAATRIKKKGEKFESLEARRKPTADKAEKKSESTEEKGDTPLEDRFTEDLSEVSGEDFRGLKNSFDDDSSPDEILDALTSKFSDPTIKDLALDYLIQTAPSDGKLKSTLIQAKHQLMSQNPQAIVGGRNVLLASETFASRANTSPSSLRSLYFQVTSSPSNCANLHQMLASYLPSEKTAVMEFLVNGMVADLKSEGPSIPPAKLQVYMTELSNLQALHSVNSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 3 - protein, 257 aa, Chlamydia trachomatis
MCIKRKKTWIAFLAVVCSFCLTGCLKEGGDSNSEKFIVGTNATYPPFEFVDKRGEVVGFDIDLAREISNKLGKTLDVREFSFDALILNLKQHRIDAVITGMSITPSRLKEILMIPYYGEEIKHLVLVFKGENKHPLPLTQYRSVAVQTGTYQEAYLQSLSEVHIRSFDSTLEVLMEVMHGKSPVAVLEPSIAQVVLKDFPALSTATIDLPEDQWVLGYGIGVASDRPALALKIEAAVQEIRKEGVLAELEQKWGLNN

SEQ ID NO: 4 - protein, 660 aa, Chlamydia trachomatis
MSEKRKSNKIIGIDLGTTNSCVSVMEGGQPKVIASSEGTRTTPSIVAFKGGETLVGIPAKRQAVTNPEKTLASTKRFIGRKFSEVESEIKTVPYKVAPNSKGDAVFDVEQKLYTPEEIGAQILMKMKETAEAYLGETVTEAVITVPAYFNDSQRASTKDAGRIAGLDVKRIIPEPTAAALAYGIDKEGDKKIAVFDLGGGTFDISILEIGDGVFEVLSTNGDTHLGGDDFDGVIINWMLDEFKKQEGIDLSKDNMALQRLKDAAEKAKIELSGVSSTEINQPFITIDANGPKHLALTLTRAQFEHLASSLIERTKQPCAQALKDAKLSASDIDDVLLVGGMSRMPAVQAVVKEIFGKEPNKGVNPDEVVAIGAAIQGGVLGGEVKDVLLLDVIPLSLGIETLGGVMTPLVERNTTIPTQKKQIFSTAADNQPAVTIVVLQGERPMAKDNKEIGRFDLTDIPPAPRGHPQIEVTFDIDANGILHVSAKDAASGREQKIRIEASSGLKEDEIQQMIRDAELHKEEDKQRKEASDVKNEADGMIFRAEKAVKDYHDKIPAELVKEIEEHIEKVRQAIKEDASTTAIKAASDELSTHMQKIGEAMQAQSASAAASSAANAQGGPNINSEDLKKHSFSTRPPAGGSASSTDNIEDADVEIVDKPE

SEQ ID NO: 5 - protein, 254 aa, Chlamydia trachomatis
MHDALQSILAIQELDIKMIRLMRVKKEHQNELAKIQALKTDIRRKVEEKEQEMEKLKDQIKGGEKRIQEISDQINKLENQQAAVKKMDEFNALTQEMTAANKERRTLEHQLSDLMDKQAGSEDLLISLKESLSSTENSSSAIEEEIRENIRKINEEGRSLLSQRTQLKETTDPELFSIYERLLNNKKDRVVVPIENRVCSGCHIALTPQHENLVRKQDHLVFCEHCSRILYWQELQSPSAEGATTKRRRRRTAV

SEQ ID NO: 6 - protein, 173 aa, Chlamydia trachomatis
MKKFLLLSLMSLSSLPTFAANSTGTIGIVNLRRCLEESALGKKESAEFEKMKNQFSNSMGKMEEELSSIYSKLQDDDYMEGLSETAAAELRKKFEDLSAEYNTAQGQYYQILNQSNLKRMQKIMEEVKKASETVRIQEGLSVLLNEDIVLSIDSSADKTDAVIKVLDDSFQNN

SEQ ID NO: 7 - protein, 130 aa, Chlamydia trachomatis
MTTESLETLVEQLSGLTVLELSQLKKLLEEKWDVTAAAPVVAVAGAAAAGDAPASAEPTEFAVILEDVPSDKKIGVLKVVREVTGLALKEAKEMTEGLPKTVKEKTSKSDAEDTVKKLQEAGAKAVAKGL

SEQ ID NO: 8 - protein, 88 aa, Chlamydia trachomatis
MKKTALLAALCSVVSLSSCCRIVDCCFEDPCAPIQCSPCESKKKDVDGGCNSCNGYVPACKPCGGDTHQDAKHGPQARGIPVDGKCRQ

SEQ ID NO: 9 - protein, 352 aa, Chlamydia trachomatis
MPKIDTCDSCVSNTELLAIRTRVTQSYNEAQTILSSIPDGIFLLSESGEILICNPQARAILGIPEDIQLVTRMFHDFFPDTFFGFSVQEALEKEVPPKTIRLTLSQELSQKEVEVFVRKNISHDFLFLLIRDRSDYRQLEQAIEKYRSISELGKIAATLAHEIRNPLTSISGFATLLKEELSSERHQRMLNVIIEGTRSLNSLVSSMLEYTKIQPLNLRSIDLQDFFSSLIPELSLTFPSCTFRRTILSPIQRSIDPDRLRCVIWNLVKNAVEASDEEIFLELHEKGFSVINTGTLPPNIQEKLFIPFFTTKPQGNGLGLAEAHKIMRLHGGDLVVSTQDNRTTFTILWTPA

SEQ ID NO: 10 - protein, 318 aa, Chlamydia trachomatis
MKVILRALCLFLVLPCGCYARVPSFEPFRGAIAPNRYTPKHSPELYFEMGDKYFQAKKFKQALLCFGMITHHFPEHALHPKAQFLVGLCYLEMGHPDLADKALTQYQELADTEYSEQLFAIKYSIAQSFANGKRKNIVPLEGFPKLLKADTDALRIFEEIVTASSDADLKASALYAKGALLFDRKEYSEAIKTLKKVSLQFPSHSLSPESFTLIAKIHCLQALQEPYNEQYLQDARMNAAALRKQHPNHPSNTEVENYIHHMCEAYASCLYSTGRFYEKKRKASSAKIYYSIALENFPDTSYVAKCNKRLERLSKQMS

SEQ ID NO: 11 - protein, 424 aa, Chlamydia trachomatis [sequence omitted]

SEQ ID NO: 12 - protein, 497 aa, Chlamydia trachomatis
MMKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSHREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVISAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVVLKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 13 - protein, 352 aa, Chlamydia trachomatis
MKKINKIVLAVGGTGGHIIPALAARETFIHEDIEVLLLGKGLAHFLGDDSEVAYCDIPSGSPFSLRVNRMFSGAKQLYKGYVAALQKIRDFTPDLAIGFGSYHSLPAMLASIRSRIPLFLHEQNIVPGKVNKLFSRFAKGVGMSFAAAGEHFHCRAEEVFLPIRKLSEQIVFPGASPVICVVGGSQGAKILNDVVPKALARIRESYSNLYVHHIVGPKGDLQAVSQVYQDAGINHTVTAFDHNMLGVLQASDLVISRSGATMLNELLWVQVPAILIPYPGAYGHQEVNAKFFTHTVGGGTMILQKYLTEESLSKQVLLALDPATSENRRKAMLSAQQKKSFKSLYQFICESL

SEQ ID NO: 14 - protein, 264 aa, Chlamydia trachomatis
MGNSGFYLYNTQNCVFADNIKVGQMTEPLKDQQIILGTTSTPVAAKMTASDGISLTVSNNPSTNASITIGLDAEKAYQLILEKLGDQILGGIADTIVDSTVQDILDKITTDPSLGLLKAFNNFPITNKIQCNGLFTPRNIETLLGGTEIGKFTVTPKSSGSMFLVSADIIASRMEGGVVLALVREGDSKPYAISYGYSSGVPNLCSLRTRIINTGLTPTTYSLRVGGLESGVVWVNALSNGNDILGITNTSNVSFLEVIPQTNA

SEQ ID NO: 15 - protein, 975 aa, Chlamydia trachomatis
MNRVIEIHAHYDQRQLSQSPNTNFLVHHPYLTLIPKFLLGALIVYAPYSFAEMELAISGHKQGKDRDTFTMISSCPEGTNYIINRKLILSDFSLLNKVSSGGAFRNLAGKISFLGKNSSASIHFKHININGFGAGVFSESSIEFTDLRKLVAFGSESTGGIFTAKEDISFKNNHHIAFRNNITKGNGGVIQLQGDMKGSVSFVDQRGAIIFTNNQAVTSSSMKHSGRGGAISGDFAGSRILFLNNQQITFEGNSAVHGGAIYNKNGLVEFLGNAGPLAFKENTTIANGGAIYTSNFKANQQTSPILFSQNHANKKGGAIYAQYVNLEQNQDTIRFEKNTAKEGGGAITSSQCSITAHNTIIFSDNAAGDLGGGAILLEGKKPSLTLIAHSGNIAFSGNTMLHITKKASLDRHNSILIKEAPYKIQLAANKNHSIHFFDPVMALSASSSPIQINAPEYETPFFSPKGMIVFSGANLLDDAREDVANRTSIFNQPVHLYNGTLSIENGAHLIVQSFKQTGGRISLSPGSSLALYTMNSFFHGNISSKEPLEINGLSFGVDISPSNLQAEIRAGNAPLRLSGSPSIHDPEGLFYENRDTAASPYQMEILLTSDKIVDISKFTTDSLVTNKQSGFQGAWHFSWQPNTINNTKQKILRASWLPTGEYVLESNRVGRAVPNSLWSTFLLLQTASHNLGDHLCNNRSLIPTSYFGVLIGGTGAEMSTHSSEEESFISRLGATGTSIIRLTPSLTLSGGGSHMFGDSFVADLPEHITSEGIVQNVGLTHVWGPLTVNSTLCAALDHNAMVRICSKKDHTYGKWDTFGMRGTLGASYTFLEYDQTMRVFSFANIEATNILQRAFTETGYNPRSFSKTKLLNIAIPIGIGYEFCLGNSSFALLGKGSIGYSRDIKRENPSTLAHLAMNDFAWTTNGCSVPTSAHTLANQLILRYKACSLYITAYTINREGKNLSNSLSCGGYVGF

SEQ ID NO: 16 - protein, 1751 aa, Chlamydia trachomatis
MKWLSATAVFAAVLPSVSGFCFPEPKELNFSRVGTSSSTTFTETVGEAGAEYIVSGNASFTKFTNIPTTDTTTPTNSNSSSSNGETASVSEDSDSTTTTPDPKGGGAFYNAHSGVLSFMTRSGTEGSLTLSEIKITGEGGAIFSQGELLFTDLTGLTIQNNLSQLSGGAIFGESTISLSGITKATFSSNSAEVPAPVKKPTEPKAQTASETSGSSSSSGNDSVSSPSSSRAEPAAANLQSHFICATATPAAQTDTETSTPSHKPGSGGAIYAKGDLTIADSQEVLFSINKATKDGGAIFAEKDVSFENITSLKVQTNGAEEKGGAIYAKGDLSIQSSKQSLFNSNYSKQGGGALYVEGDINFQDLEEIRIKYNKAGTFETKKITLPKAQASAGNADAWASSSPQSGSGATTVSNSGDSSSGSDSDTSETVPATAKGGGLYTDKNLSITNITGIIEIANNKATDVGGGAYVKGTLTCENSHRLQFLKNSSDKQGGGIYGEDNITLSNLTGKTLFQENTAKEEGGGLFIKGTDKALTMTGLDSFCLINNTSEKHGGGAFVTKEISQTYTSDVETIPGITPVHGETVITGNKSTGGNGGGVCTKRLALSNLQSISISGNSAAENGGGAHTCPDSFPTADTAEQPAAASAATSTPESAPVVSTALSTPSSSTVSSLTLLAASSQASPATSNKETQDPNADTDLLIDYVVDTTISKNTAKKGGGIYAKKAKMSRIDQLNISENSATEIGGGICCKESLELDALVSLSVTENLVGKEGGGLHAKTVNISNLKSGFSFSNNKANSSSTGVATTASAPAAAAASLQAAAAAVPSSPATPTYSGVVGGAIYGEKVTFSQCSGTCQFSGNQAIDNNPSQSSLNVQGGAIYAKTSLSIGSSDAGTSYIFSGNSVSTGKSQTTGQIAGGAIYSPTVTLNCPATFSNNTASMATPKTSSEDGSSGNSIKDTIGGAIAGTAITLSGVSRFSGNTADLGAAIGTLANANTPSATSGSQNSITEKITLENGSFIFERNQANKRGAIYSPSVSIKGNNITFNQNTSTHDGSAIYFTKDATIESLGSVLFTGNNVTATQASSATSGQNTNTANYGAAIFGDPGTTQSSQTDAILTLLASSGNITFSNNSLQNNQGDTPASKFCSIAGYVKLSLQAAKGKTISFFDCVHTSTKKIGSTQNVYETLDINKEENSNPYTGTIVFSSELHENKSYIPQNAILHNGTLVLKEKTELHVVSFEQKEGSKLIMKPGAVLSNQNIANGALVINGLTIDLSSMGTPQAGEIFSPPELRIVATTSSASGGSGVSSSIPTNPKRISAAAPSGSAATTPTMSENKVFLTGDLTLIDPNGNFYQNPMLGSDLDVPLIKLPTNTSDVQVYDLTLSGDLFPQKGYMGTWTLDSNPQTGKLQARWTFDTYRRWVYIPRDNHFYANSILGSQNSMIVVKQGLINNMLNNARFDDIAYNNFWVSGVGTFLAQQGTPLSEEFSYYSRGTSVAIDAKPRQDFILGAAFSKMVGKTKAIKKMHNYFHKGSEYSYQASVYGGKFLYFLLNKQHGWALPFLIQGVVSYGHIKHDTTTLYPSIHERNKGDWEDLGWLADLRISMDLKEPSKDSSKRITVYGELEYSSIRQKQFTEIDYDPRHFDDCAYRNLSLPVGCAVEGAIMNCNILMYNKLALAYMPSIYRNNPVCKYRVLSSNEAGQVICGVPTRTSARAEYSTQLYLGPFWTLYGNYTIDVGMYTLSQMTSCGARMIF

SEQ ID NO: 17 - protein, 1770 aa, Chlamydia trachomatis
MKFMSATAVFAAALSSVTEASSIQDQIKNTDCNVSKLGYSTSQAFTDMMLADNTEYRAADSVSFYDFSTSSRLPRKHLSSSSEASPTTEGVSSSSSGETDEKTEEELDNGGIIYAREKLTISESQDSLSNQSIELHDNSIFFGEGEVIFDHRVALKNGGAIYGEKEVVFENIKSLLVEVNIAVEKGGSVYAKERVSLENVTEATFSSNGGEQGGGGIYSEQDMLISDCNNVHFQGNAAGATAVKQCLDEEMIVLLAECVDSLSEDTLDSTPETEQTESNGNQDGSSETEDTQVSESPESTPSPDDVLGKGGGIYTEKSLTITGITGTIDFVSNIATDSGAGVFTKENLSCTNTNSLQFLKNSAGQHGGGAYVTQTMSVTNTTSESITTPPLIGEVIFSENTAKGHGGGICTNKLSLSNLKTVTLTKNSAKESGGAIFTDLASIPITDTPESSTPSSSSPASTPEVVASAKINRFFASTAKPAAPSLTEAESDQTDQTETSDTNSDIDVSIENILNVAINQNTSAKKGGAIYGKKAKLSRINNLELSGNSSQDVGGGLCLTESVEFDAIGSLLSHYNSAAKEGGAIHSKTVTLSNLKSTFTFADNTVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGQGIFSGNKAIDNTTEGSSSKSDVLGGAVYAKTLFNLDSGSSRRTVTFSGNTVSSQSTTGQVAGGAIYSPTVTIATPVVFSKNSATNNANNTTDTQRKDTFGGAIGATSAVSLSGGAHFLENVADLGSAIGLVPGTQNTETVKLESGSYYFEKNKALKRATIYAPVVSIKAYTATFNQNRSLEEGSAIYFTKEASIESLGSVLFTGNLVTLTLSTTTEGTPATTSGDVTKYGAAIFGQIASSNGSQTDNLPLKLIASGGNICFRNNEYRPTSSDTGTSTFCSIAGDVKLTMQAAKGKTISFFDAIRTSTKKTGTQATAYDTLDINKSEDSETVNSAFTGTILFSSELHENKSYIPQNVVLHSGSLVLKPNTELHVISFEQKEGSSLVMTPGSVLSNQTVADGALVINNMTIDLSSVEKNGIAEGNIFTPPELRIIDTTTGGSGGTPSTDSESNQNSDDTEEQNNNDASNQGESANGSSSPAVAAAHTSRTRNFAAAATATPTTTPTATTTTSNQVILGGEIKLIDPNGTFFQNPALRSDQQISLLVLPTDSSKMQAQKIVLTGDIAPQKGYTGTLTLDPDQLQNGTISVLWKFDSYRQWAYVPRDNHFYANSILGSQMLMVTVKQGLLNDKMNLARFEEVSYNNLWISGLGTMLSQVGTPTSEEFTYYSRGASVALDAKPAHDVIVGAAFSKMIGKTKSLKRENNYTHKGSEYSYQASVYGGKPFHFVINKKTEKSLPLLLQGVISYGYIKHDTVTHYPTIRERNKGEWEDLGWLTALRVSSVLRTPAQGDTKRITVYGELEYSSIRQKQFTETEYDPRYFDNCTYRNLAIPMGLAFEGELSGNDILMYNRFSVAYMLSIYRNSPTCKYQVLSSGEGGEIICGVPTRNSARGEYSTQLYLGPLWTLYGSYTIEADAHTLAHMMNCGARMTF

SEQ ID NO: 18 - protein, 1531 aa, Chlamydia trachomatis
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 19 - protein, 964 aa, Chlamydia trachomatis
MKKAFFFFLIGNSLSGLAREVPSRIFLMPNSVPDPTKESLSNKISLTGDTHNLTNCYLDNLRYILAILQKTPNEGAAVTITDYLSFFDTQKEGIYFAKNLTPESGGAIGYASPNSPTVEIRDTIGPVIFENNTCCRLFTWRNPYAADKIREGGAIHAQNLYINHNHDVVGFMKNFSYVQGGAISTANTFVVSENQSCFLFMDNICIQTNTAGKGGAIYAGTSNSFESNNCDLFFINNACCAGGAIFSPICSLTGNRGNIVFYNNRCFKNVETASSEASDGGAIKVTTRLDVTGNRGRIFFSDNITKNYGGAIYAPVVTLVDNGPTYFINNIANNKGGAIYIDGTSNSKISADRHAIIFNENIVTNVTNANGTSTSANPPRRNAITVASSSGEILLGAGSSQNLIFYDPIEVSNAGVSVSFNKEADQTGSVVFSGATVNSADFHQRNLQTKTPAPLTLSNGFLCIEDHAQLTVNRFTQTGGVVSLGNGAVLSCYKNGTGDSASNASITLKHIGLNLSSILKSGAEIPLLWVEPTNNSNNYTADTAATFSLSDVKLSLIDDYGNSPYESTDLTHALSSQPMLSISEASDNQLQSENIDFSGLNVPHYGWQGLWTWGWAKTQDPEPASSATITDPQKANRFHRTLLLTWLPAGYVPSPKHRSPLIANTLWGNMLLATESLKNSAELTPSGHPFWGITGGGLGMMVYQDPRENHPGFHMRSSGYSAGMIAGQTHTFSLKFSQTYTKLNERYAKNNVSSKNYSCQGEMLFSLQEGFLLTKLVGLYSYGDHNCHHFYTQGENLTSQGTFRSQTMGGAVFFDLPMKPFGSTHILTAPFLGALGIYSSLSHFTEVGAYPRSFSTKTPLINVLVPIGVKGSFMNATHRPQAWTVELAYQPVLYRQEPGIAAQLLASKGIWFGSGSPSSRHAMSYKISQQTQPLSWLTLHFQYHGFYSSSTFCNYLNGEIALRF

SEQ ID NO: 20 - protein, 1034 aa, Chlamydia trachomatis
MIKRTSLSFACLSFFYLSTISILQANETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGRLSIVDENYESVYDSMDLSRGKAEQLILSIETTNDGQLDSNWQSSLNTSLLSPPHYGYQGLWTPNWITTTYTITLNNNSSAPTSATSIAEQKKTSETFTPSNTTTASIPNIKASAGSGSGSASNSGEVTITKHTLVVNWAPVGYIVDPIRRGDLIANSLVHSGRNMTMGLRSLLPDNSWFALQGAATTLFTKQQKRLSYHGYSSASKGYTVSSQASGAHGHKFLLSFSQSSDKMKEKETNNRLSSRYYLSALCFEHPMFDRIALIGAAACNYGTHNMRSFYGTKKSSKGKFHSTTLGASLRCELRDSMPLRSIMLTPFAQALFSRTEPASIRESGDLARLFTLEQAHTAVVSPIGIKGAYSSDTWPTLSWEMELAYQPTLYWKRPLLNTLLIQNNGSWVTTNTPLAKHSFYGRGSHSLKFSHLKLFANYQAEVATSTVSHYINAGGALVF

SEQ ID NO: 21 - protein, 1013 aa, Chlamydia trachomatis
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 22 - protein, 1016 aa, Chlamydia trachomatis
MPFSLRSTSFCFLACLCSYSYGFASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVSRDVGFVASLHALGDYILNYTQDDRDGFLARYGGFQATAASHYENGSIFGVAFGQLYGQTKSRMYYSKDAGNMTMLSCFGRSYVDIKGTETVMYWETAYGYSVHRMHTQYFNDKTQKFDHSKCHWHNNNYYAFVGAEHNFLEYCIPTRQFARDYELTGFMRFEMAGGWSSSTRETGSLTRYFARGSGHNMSLPIGIVAHAVSHVRRSPPSKLTLNMGYRPDIWRVTPHCNMEIIANGVKTPIQGSPLARHAFFLEVHDTLYIHHFGRAYMNYSLDARRRQTAHFVSMGLNRIF

SEQ ID NO: 23 - protein, 878 aa, Chlamydia trachomatis
MRPDHMNFCCLCAAILSSTAVLFGQDPLGETALLTKNPNHVVCTFFEDCTMESLFPALCAHASQDDPLYVLGNSYCWFVSKLHITDPKEALFKEKGDLSIQNFRFLSFTDCSSKESSPSIIHQKNGQLSLRNNGSMSFCRNHAEGSGGAISADAFSLQHNYLFTAFEENSSKGNGGAIQAQTFSLSRNVSPISFARNRADLNGGAICCSNLICSGNVNPLFFTGNSATNGGAICCISDLNTSEKGSLSLACNQETLFASNSAKEKGGAIYAKHMVLRYNGPVSFINNSAKIGGAIAIQSGGSLSILAGEGSVLFQNNSQRTSDQGLVRNAIYLEKDAILSSLEARNGDILFFDPIVQESSSKESPLPSSLQASVTSPTPATASPLVIQTSANRSVIFSSERLSEEEKTPDNLTSQLQQPIELKSGRLVLKDRAVLSAPSLSQDPQALLIMEAGTSLKTSSDLKLATLSIPLHSLDTEKSVTIHAPNLSIQKIFLSNSGDENFYENVELLSKEQNNIPLLTLSKEQSHLHLPDGNLSSHFGYQGDWTFSWKDSDEGHSLIANWTPKNYVPHPERQSTLVANTLWNTYSDMQAVQSMINTIAHGGAYLFGTWGSAVSNLFYAHDSSGKPIDNWHHRSLGYLFGISTHSLDDHSFCLAAGQLLGKSSDSFITSTETTSYIATVQAQLATPLMKISAQACYNESIHELKTKYRSFSKEGFGSWHSVAVSGEVCASIPIVSNGSGLFSSFSIFSKLQGFSGTQDGFEESSGEIRSFSASSFRNISLPMGITFEKKSQKTRNYYYFLGAYIQDLKRDVESGPVVLLKNAVSWDAPMANLDSRAYMFRLTNQRALHRLQTLLNVSYVLRGQSHSYSLDLGTTYRF

SEQ ID NO: 24 - protein, 393 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 25 - protein, 10 aa, Chlamydia trachomatis
CSFIGGITYL

SEQ ID NO: 26 - protein, 9 aa, Chlamydia trachomatis
SFIGGITYL

SEQ ID NO: 27 - protein, 9 aa, Chlamydia trachomatis
SIIGGITYL

SEQ ID NO: 28 - protein, 298 aa, Chlamydia trachomatis
MASICGRLGSGTGNALKAFFTQPNNKMARVVNKTKGMDKTIKVAKSAAELTANILEQAGGAGSSAHITASQVSKGLGDARTVVALGNAFNGALPGTVQSAQSFFSHMKAASQKTQEGDEGLTADLCVSHKRRAAAAVCSIIGGITYLATFGAIRPILFVNKMLAKPFLSSQTKANMGSSVSYIMAANHAASVVGAGLAISAERADCEARCARIAREESLLEVPGEENACEKKVAGEKAKTFTRIKYALLTMLEKFLECVADVFKLVPLPITMGIRAIVAAGCTFTSAIIGLCTFCARA

SEQ ID NO: 29 - protein, 544 aa, Chlamydia trachomatis
MVAKNIKYNEEARKKIQKGVKTLAEAVKVTLGPKGRHVVIDKSFGSPQVTKDGVTVAKEVELADKHENMGAQMVKEVASKTADKAGDGTTTATVLAEAIYTEGLRNVTAGANPMDLKRGIDKAVKVVVDQIRKISKPVQHHKEIAQVATISANNDAEIGNLIAEAMEKVGKNGSITVEEAKGFETVLDIVEGMNFNRGYLSSYFATNPETQECVLEDALVLIYDKKISGIKDFLPVLQQVAESGRPLLIIAEDIEGEALATLVVNRIRGGFRVCAVKAPGFGDRRKAMLEDIAILTGGQLISEELGMKLENANLAMLGKAKKVIVSKEDTTIVEGMGEKEALEARCESIKKQIEDSSSDYDKEKLQERLAKLSGGVAVIRVGAATEIEMKEKKDRVDDAQHATIAAVEEGILPGGGTALIRCIPTLEAFLPMLTNEDEQIGARIVLKALSAPLKQIAANAGKEGAIIFQQVMSRSANEGYDALRDAYTDMLEAGILDPAKVTRSALESAASVAGLLLTTEALIAEIPEEKPAAAPAMPGAGMDY

SEQ ID NO: 30 - protein, 553 aa, Chlamydia trachomatis
MRIGDPMNKLIRRAVTIFAVTSVASLFASGVLETSMAESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 31 - protein, 326 aa, Chlamydia trachomatis
MFRYTLSRSLFFILALFFCSACDSRSMITHGLSGRDANEIVVLLVSKGVAAQKVPQAASSTGGSGEQLWDISVPAAQITEALAILNQAGLPRMKGTSLLDLFAKQGLVPSEMQEKIRYQEGLSEQMATTIRKMDGIVDASVQISFSPEEEDQRPLTASVYIKHRGVLDNPNSIMVSKIKRLVASAVPGLCPENVSVVSDRASYSDITINGPWGLSDEMNYVSVWGIILAKHSLTKFRLVFYFLILLLFILSCGLLWVIWKTHTLISALGGTKGFFDPAPYSQLSFTQNKPAPKETPGAAEGAEAQTASEQPSKENAEKQEENNEDA

SEQ ID NO: 32 - protein, 188 aa, Chlamydia trachomatis
MRKTIFKAFNLLFSLLFLSSCSYPCRDWECHGCDSARPRKSSFGFVPFYSDEEIQQAFVEDFDSKEEQLYKTSAQSTSFRNITFATDSYSIKGEDNLTILASLVRHLHKSPKATLYIEGHTDERGAAAYNLALGARRANAVKQYLIKQGIAADRLFTISYGKEHPVHPGHNELAWQQNRRTEFKIHAR

SEQ ID NO: 33 - protein, 243 aa, Chlamydia trachomatis
MKNILSWMLMFAVALPIVGCDNGGGSQTSATEKSMVEDSALTDNQKLSRTFGHLLSRQLSRTEDFSLDLVEVIKGMQSEIDGQSAPLTDTEYEKQMAEVQKASFEAKCSENLASAEKFLKENKEKAGVIELEPNKLQYRVVKEGTGRVLSGKPTALLHYTGSFIDGKVFDSSEKNKEPILLPLTKVIPGFSQGMQGMKEGEVRVLYIHPDLAYGTAGQLPPNSLLIFEVKLIEANDDNVSVTE

SEQ ID NO: 34 - protein, 432 aa, Chlamydia trachomatis
MKKYFYKGFVGALLLACGSTNLAFAQASSMDSQLWSVEDLDSYLSSKGFVETRKRDGVLRLAGDVRARWIYAKEDLETTQTPAKPMLPTNRYRSEFNLYVDYTAANSWMTSKMNWVTIAGGESSAAGLDINRAFLGYRFYKNPETQAEVFAEIGRSGLGDIFDSDVQFNSNFDGIHLYAARRISEKLPFTMIVHGGPFVVNMAEKEYAWVVEAILNKLPGNFVVKTSVVDWNTLTAKTNDPADASAAQPAKPNTKYDYLVWQWLVGKSTAMPWFNGQTKNLYTYGAYLFNPLAEIPENWKQSTTPTTKITNGKENHAWFIGCSLGGVRRAGDWSATVRYEYVEALAIPEIDVAGIGRGNQMKYWFAQAIKQGLDPKESNGFTNYKGVSYQFVMGLTDSVSFRAYAAYSKPANDNLGSDFTYRKYDLGLISSF

SEQ ID NO: 35 - protein, 441 aa, Chlamydia trachomatis
MWLIVASTLLACLAMALVFKAYRHVISFRSYVNQVIRDVRLSVDLKEWAVAEMRLAPILKKRQYRRKYLFEYIRILRELERFEEAEKLLGEAKKLKLAGAHFFLEVAHKAFRHGAYKEAAHAFSLLSAELMGEREVARYTISLVYLGEVDAACRIIEPWIGPLAHQEVFISVGHIYFATKRYADAIDFYRRARSLGSCPIDVLYNLAHSLRICGQYVDAGMLFRELLGDPVYKDEAMFNIGLCEQKLGNSKKALLIYQNSELWVRGDALMMRYAALAAADQQDYQLAEHCWTLAFRCQSYADDWNCCVHYGLALCHLKKYAEAEKVYLRVIQKTPDCLVACKALAWLAGVGHATMISAREGIAYAKRALQIKRSPEVLELLSACEAREGNFDVAYDIQAILAERDTTAKERERRSQILKNLRQKLPIDQQHIVEVSLLLAA

SEQ ID NO: 36 - protein, 393 aa, Chlamydia trachomatis
MLVESQLGLEDVLEAFSERNFDIQSKSFIESFQDKKLRRTVIQRFLHHPLLHIHDIARAAYLLAALEEGVDLGYQFLCMHQTQSGAALLFRRAGFLWGGLPYPGEHAEMAMLLSRIAEFYDTSYEQVQKMIAFQHALFSHERNIFPALWSQEGSRSNQEKTAVSKLLFCQKEARIEDQFTLTDMSLGFWMRRTPSFSAYVSGSGCKSGVGAFLIGDVGVLNYGPCVGDPGECLGFGLCGQVKEFSCQEKDEEVSISFAGALSQPSSRRTGFSYLQDALFSTNSCYCIDITEQKCHVASSLDRENQDAFFAIFCKGSQCQVCNGPKLRTGSPDSYKGPAYDVLIKGEKETVRILSSSPHMEIFSLQGKDRFWGSNFLINLPYTQNSINILFEKA

SEQ ID NO: 37 - protein, 316 aa, Chlamydia trachomatis
MGKFFASYLLILAPFFLQSCSAPSRTTLEGVRMTIPYRIVFGEALSPDAFQQAQKEIDRVFDHIDQTFNNWNPLSEISRINRTTKQTPIPLSPALFAFLCEIDHFHAFSDGRFDPTLGALKSLWLLHLKSHTIPSQELQHLYKHSSGWHLISLDKTQQTLRKLSPLVQLDLCGTVKGFAVDLLGTACAQFCQNYYVEWGGEIKTKGKHPSGRSWAVASSATPEILHLHDHAIATSGSQYQRWHVDNKTYTHILDPLTGTPLEDSSHPILAVSVINESCAFADAMATALTTFSSKQEALDWANKKHLCAYITDKNVS

SEQ ID NO: 38 - protein, 340 aa, Chlamydia trachomatis
MSSKLVNYLRLTFLSFLGIASTSLDAMPAGNPAFPVIPGINIEQKNACSFDLCNSYDVLSALSGNLKLCFCGDYIFSEEAQVKDVPVVTSVTTAGVGPSPDITSTTKTRNFDLVNCNLNTNCVAVAFSLPDRSLSAIPLFDVSFEVKVGGLKQYYRLPMNAYRDFTSEPLNSESEVTDGMIEVQSNYGFVWDVSLKKVIWKDGVSFVGVGADYRHASCPIDYIIANSQANPEVFIADSDGKLNFKEWSVCVGLTTYVNDYVLPYLAFSIGSVSRQAPDDSFKKLEDRFTNLKFKVRKITSSHRGNICIGATNYVADNFFYNVEGRWGSQRAVNVSGGFQF

SEQ ID NO: 39 - protein, 148 aa, Chlamydia trachomatis
MFQPETVPSNRSTETTPQNIEVYNDRNFTNHTTEDVIRIGERLQRQFYNMTEESRVPFTTSPSHHTGNWKTAFLYNLSQVVAHIFPSTVQPIRVKPTRIPPSPTPPPEGTTTAETSTSENKVTTISKEQEVTTKPLLVRERRSLLHSQ

SEQ ID NO: 40 - protein, 560 aa, Chlamydia trachomatis
MSISGSGNVSPATPDFDPSILMGRQAASAHAAKEASGASKATETSAAEQQALISSGTELDYVTDLQQSEGKYKKTLDKTSKSPKTKLKGNFSKVRAGTKGFLTGFGTRASRISARKAENNGEGMSMIPSQMEYVKKKGNRVSPEMQNFYLGASGLWSPTSDVSSITENCLGATALSTTPLLTTMQDPVSIEHLSSGEITALASFNPNVRTASLNEQTINAWTEARLGGEMVSTLLDPNIETSSLLRRAPTVSNEGMVDVSDMGNQTTSLSMEGLVNTVVDDPASAEEEKKTGELSLEEMAAMAKMMAALLSSGQGMAVFIASSTPSSGLTQFPEPKFSGTIPHHFSKKEDNETIWGLDSQIGSIAFDTRRENNASPLPTTSLHEEASYRFPVGEAPLDVNEIPFAVQHSTVFSKETANTEQALIQNESLGEIPVSAEVVGQDTVSSAYQFPSHLGMAVLASVPLSTEDYKTAVEHRKGPGGPPDPLIYQYRNVAVDPAIIFQSPSPFSVSSRFSVQGKPEAVAVYNDDQEEAAGGNRDSDEGKDQEQDKTRETEDAGGDS

SEQ ID NO: 41 - protein, 236 aa, Chlamydia trachomatis
MLSKFCKLSLSAILLINTLAPSETFSEEGTSGFLGRMKSWILKDKTILSTTEESQTSAIEKVSDLLSWKRYDYTQESGFAIQFPESPEHSEQVIEVPQSDLAIRYDTYVAETPSDSTVYVVSIWEYPEKIDISRPELNLQEGFAGMLYALPESQVLYLKATALQGHKALEFWIACDDVYFRGMLVSVNHTLYQVFMVYKGRSPEILDKEYSTFIQSFKVTKVRNSKKMDIRKRVSL

SEQ ID NO: 42 - protein, 536 aa, Chlamydia trachomatis
MNDTKNNISSSFWNPNKVVTKVLLKVSETGIESTPGIVKHNQLITQSENPTDPTDAVTFKYLKENYTKENDPNPGFLPTTGGTMTGDIDMQGNNVTDIVMYTNGQQNPTDDSAVTIGYLNEKADEIKSNDQITTAVAGLSNINSQISTLHQLLGIAEDPDTVTNPDLLKTSGGTVYEDIDMSSNTVSDLGTPTNKDTKSAINVEFVQAKITSPQMAFLKNNDTNLSNITVSEYFNWLQDPTQAPTPEPDPDPEPAPEPEPDTSDSSGSGSENPADPAPTNPSDSNAQNNPTPSSNGATASIRKLAATTTTVPTDTEIAPAAEDPNLPNTTFSEKSPLWEEFFSFSDSSRSEMVIQKTGILTFSMQGTWENPSSSQTPSTDPISLELTVTPPTTDTPPESPPSPPEAPAPEATPSPTNNNLTASITKTFSRKYNLSATPSPTPTTPTEPTTITKTLSLSSGQSCTLQIPVQATRSVLKLKYVNPNNNSSGGSSGSGGSSQPETTPTGITLQSFSWSLVLTPGEITKATSTPSTPSQP

SEQ ID NO: 43 - protein, 404 aa, Chlamydia trachomatis
MSVQGSSSLKYSDLFKPPEPTSSTDSSKEPPKESAWKVVSHSRGRRRARSNPSPHTSQNTPSPKDSSLVARTDKAATDIFNSAKHKAIETTKRSDQQSRSLHILHLLAENPEPIVFHSAHQTNHNDPQRMLCDAILQANRIITMRIFNIGSPEIIRALIRAVRRNIPVVVSAWNFPNLSNWDRESELCVELRGNPQICLHKKTTLIDNQLTIIGTANYTKSSFFKDINLTALIQNPALYSLILSDTRGSVSIGSQTISYYPLPFPQSNTKILPIIQEIQKAQRTIKIAMNIFSHTEIFLALEQARLRGVTITIVINKKESAHTLDILHRISALLLLKSVTTVDSLHAKICLIDNQTLIFGSPNWTYHGMHKNLEDLLIVTPLTPKQIHSIQEIWAFLLKNSSPV

SEQ ID NO: 44 - protein, 245 aa, Chlamydia trachomatis
MDRSPLFLIIMGAPGSGKGTQSKLLASQLSLLHISSGDLLRDAVSKDTPLSQEIKSYLDQGKLLPDTLVWKLVHEKLDEFQQDTLLRRLSFLSRSENSAILDGFPRTVTQAKLLHEFLSSYFPNYKVILLDISDEEVLNRLTSRYICPACQGIYNEQQGFSSCPKCSVELIRRSDDTLEVILDRIQTYKQETQPVLDYYTEKQKLITIDANAPTQQVFQSILDSLSASLVYQERDCCNCDCDDED

SEQ ID NO: 45 - protein, 810 aa, Chlamydia trachomatis
MTKPSFLYVIQPFSVFNPRLGRFSTDSDTYIEEENRLASFIESLPLEIFDIPSFMETAISNSPYILSWETTKDGALFTILEPKLSACAATCLVAPSIQMKSDAELLEEIKQALLRSSHDGVKYRITRESFSPEKKTPKVALVDDDIELIRNVDFLGRAVDIVKLDPINILNTVSEENILDYSFTRETAQLSADGRFGIPPGTKLFPKPSFDVEISTSIFEETTSFTRSFSASVTFSVPDLAATMPLQSPPMVENGQKEICVIQKHLFPSYSPKLVDIVKRYKREAKILINKLAFGMLWRHRAKSQILTEGSVRLDLQGFTESKYNYQIQVGSHTIAAVLIDMDISKIQSKSEQAYAIRKIKSGFQRSLDDYHIYQIERKQTFSFSPKHRSLSSTSHSEDSDLDLSEAAAFSGSLTCEFVKKSTQHAKNTVTCSTAAHSLYTLKEDDSSNPSEKRLDSCFRNWIENKLSANSPDSWSAFIQKFGTHYIASATFGGIGFQVLKLSFEQVEDLHSKKISLETAAANSLLKGSVSSSTESGYSSYSSTSSSHTVFLGGTVLPSVHDERLDFKDWSESVHLEPVPIQVSLQPITNLLVPLHFPNIGAAELSNKRESLQQAIRVYLKEHKVDEQGERTTFTSGIDNPSSWFTLEAAHSPLIVSTPYIASWSTLPYLFPTLRERSSATPIVFYFCVDNNEHASQKILNQSYCFLGSLPIRQKIFGSEFASFPYLSFYGNAKEAYFDNTYYPTRCGWIVEKLNTTQDQFLRDGDEVRLKHVSSGKYLATTPLKDTHGTLTRTTNCEDAIFIIKKSSGY

SEQ ID NO: 46 - protein, 173 aa, Chlamydia trachomatis; identical to SEQ ID NO: 6

SEQ ID NO: 47 - protein, 194 aa, Chlamydia trachomatis
MFKRPAKNFFDEVQTLYEDSGANSTSYSIYPQRTERLENHSNIFEPAKPAETRLLSQEEHSQWTDQQEELATQESSFPEEPETTLGEGVSFKGELTFERLLRIDGTFEGILVSKGKIIVGPQGYVKANIELEEAVIAGVVEGNITVTGRVSLQGRAMVTGDIQAGSLCVDEGVRLCGYVSIQGAPSNEQEEIDS

SEQ ID NO: 48 - protein, 155 aa, Chlamydia trachomatis
MRAVLHLEHKRYFQNHGHILFEGLAPVSDCKQLEAELKLFLKEVAVVKDRHLQRWRENVHRTLPEVQMIVKRVRLDHLAAELTHRSRVALVRDLWVQKQEEIFFDDCDCSVLLCLSGEKAGWGLFFSGEYPQDVFNWGAGDTAIILRFSSAGFPN

SEQ ID NO: 49 - protein, 442 aa, Chlamydia trachomatis
MQAAHHHYHRYTDKLHRQNHKKDLISPKPTEQEACNTSSLSKELIPLSEQRGLLSPICDFISERPCLHGVSVRNLKQALKNSAGTQIALDWSILPQWFNPRVSHAPKLSIRDFGYSAHQTVTEATPPCWQNCFNPSAAVTIYDSSYGKGVFQISYTLVRYWRENAATAGDAMMLAGSINDYPSRQNIFSQFTFSQNFPNERVSLTIGQYSLYAIDGTLYNNDQQLGFISYALSQNPTATYSSGSLGAYLQVAPTASTSLQIGFQDAYNISGSSIKWSNLTKNRYNFHGFASWAPRCCLGSGQYSVLLYVTRQVPEQMEQTMGWSVNASQHISSKLYVFGRYSGVTGHVFPINRTYSFGMASANLFNRNPQDLFGIACAFNNVHLSASPNTKRKYETVIEGFATIGCGPYLSFAPDFQLYLYPALRPNKQSARVYSVRANLAI

SEQ ID NO: 50 - protein, 975 aa, Chlamydia trachomatis; identical to SEQ ID NO: 15

SEQ ID NO: 51 - protein, 696 aa, Chlamydia trachomatis
MIDKIIRTILVLSLFLLYWSSDLLEKDVKSIKRELKALHEDVLELVRISHQQKNWVQSTDFSVSPEISVLKDCGDPAFPNLLCEDPYVEKVVPSLLKEGFVPKGILRTAQVGRPDNLSPFNGFVNIVRFYELCVPNLAVEHVGKYEEFAPSLALKIEEHYVEDGSGDKEFHIYLRPNMFWEPIDPTLFPKNITLADSFLRPHPVTAHDVKFYYDVVMNPYVAEMRAVAMRSYFEDMVSVRVENDLKLIVRWRAHTVRNEQGEEEKKVLYSAFANTLALQPLPCFVYQHFANGEKIVPEDSDPDTYRKDSVWAQNFSSHWAYNYIVSCGAFRFAGMDDEKITLVRNPNYHNPFAALVEKRYIYMKDSTDSLFQDFKAGKVDIAYFPPNHVDNLASFMQTSAYKEQAARGEAILEKNSSDRSYSYIGWNCLSLFFNNRSVRQAMNMLIDRDRIIEQCLDGRGVSVSGPFSLCSPSYNRDVEGWQYSPEEAARKLEEEGWIDADGDGIREKVIDGVVVPFRFRLCYYVKSVTARTIAEYVATVCKEVGIECCLLGLDMADYSQALEEKNFDAILSGWCLGTPPEDPRALWHSEGALEKGSANAVGFCNEEADRIIEQLSYEYDSNKRQALYHRFHEVIHEESPYAFLYSRQYSLVYKEFVKNIFVPTEHQDLIPGAQDETVNLSMLWVDKEEGRCSAIS

SEQ ID NO: 52 - protein, 194 aa, Chlamydia trachomatis
MLKMFWLNSLVFFSLLLSACGYTVLSPHYVEKKFSLSEGIYVCPIEGDSLGDLVSSLSYELEKRGLHTRSQGTSSGYVLKVSLFNETDENIGFAYTPQKPDEKPVKHFIVSNEGRLALSAKVQLIKNRTQEILVEKCLRKSVTFDFQPDLGTANAHQLALGQFEMHNEAIKSASRILYSQLAETIVQQVYYDLF

SEQ ID NO: 53 - protein, 167 aa, Chlamydia trachomatis
MSRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRA

SEQ ID NO: 54 - protein, 144 aa, Chlamydia trachomatis
MKNNSAQKIIDSIKQILSIYKIDFEPSFGATLTDDNDLDYQMLIEKTQEKIQELDKRSQEILQQTGMTREQMEVFANNPDNFSPEEWRALENIRSSCNEYKKETEELIKEVTNDIGHSSHKSPTPKKTKSSSQKKSKKKNWIPL

SEQ ID NO: 55 - protein, 307 aa, Chlamydia trachomatis
MRKIILCSPRGFCAGVIRAIQTVEVALEKWGRPIYVKHEIVHNRHVVDKLREKGAIFIEDLQEVPRNSRVIFSAHGVPPSVREEAEERGLIAIDATCGLVTKVHSAVKMYAKKGYHIILIGKRKHVEIIGIRGEAPDQITVVENIAEVEALPFSAQDPLFYVTQTTLSMDDAADIVAALKARYPRIFTLPSSSICYATQNRQGALRNILPQVDFVYVIGDTQSSNSNRLREVAERRGVTARLVNHPDEVTEEILQYSGNIGITAGASTPEDVVQACLMKLQELIPDLSIEMDLFVEEDTVFQLPKEL

SEQ ID NO: 56 - protein, 283 aa, Chlamydia trachomatis
MELNKTSESLFSAKIDHNHPRTEAHEPRDQREVRVFSLEGRSSTRQEKADRMPGRTSSRQESSKGSEEGAVHESTAGVSSKEEEESKGDGFFTGGNPTSGMALVETPMAVVSEAMVETSTMTVSQVDLQWVEQLVTSTVESLLVADIDGKQLVEIVLDNSNTVPAAFCGANLTLVQTGEEISVSFSNFVDQAQLTEATQLVQQNPKQLVSLVESLKARQLNLTELVVGNVAVSLPTIEKIETPLHMIAATIRHHDQEGDQEGEGRQDQHQGQHQEKKVEEAHI

SEQ ID NO: 57 - protein, 242 aa, Chlamydia trachomatis
MKVKINDQFICISPYISARWNQIAFIESCDGGTEGGITLKLHLIDGETVSIPNLGQAIVDEVFQEHLLYLESTAPQKNKEEEKISSLLGAVQQMAKGCEVQVFSQKGLVSMLLGGAGSINVLLQHSPEHKDHPDLPTDLLERIAQMMRSLSIGPTSILAKPEPHCNCLHCQIGRATVEEEDAGVSDEDLTFRSWDISQSGEKMYTVTDPLNPEEQFNVYLGTPIGCTCGQPYCEHVKAVLYT

SEQ ID NO: 58 - protein, 433 aa, Chlamydia trachomatis
MLIFALSFGADACLCAADLSKAKVEASVGDRAAFSPFTGEIKGNRVRLRLAPHTDSFIIKELSKGDCLAVLGESKDYYVVAAPEGVRGYVFRTFVLDNVIEGEKVNVRLEPSTSAPILARLSKGTVVKTLGAAQGKWIEIALPKQCVFYVAKNFVKNVGALDLYNQKEGQKKLALDLLSSAMDFADAELQKKIEDIDLDAIYKKMNLAQSEEFKDVPGLQSLVQKALERVQEAFLAKSLEKSSVKVPEIRHKVLEEIAVVSPAVEETPVVTKTEEQKVTTVPVPAPAVVTEPAQDLSSVKGSLLSHYIRKKGFVKASPVIEGRESFERSLFAVWLSLQPEEIRHQLTMESFYRDEQKKKRVLTGELEVYPHIVKNNPGDYLLKNGEDVVAFVYATSIDLSKWLGKSVVLECVSRPNNHFAFPAYIVLSVKEGA

SEQ ID NO: 59 - protein, 167 aa, Chlamydia trachomatis; identical to SEQ ID NO: 53

SEQ ID NO: 60 - protein, 194 aa, Chlamydia trachomatis; identical to SEQ ID NO: 52

SEQ ID NO: 61 - protein, 1005 aa, Chlamydia trachomatis
MTNSISGYQPTVTTSTSSTTSASGASGSLGASSVSTTANATVTQTANATNSAATSSIQTTGETVVNYTNSASAPNVTVSTSSSSTQATATSNKTSQAVAGKITSPDTSESSETSSTSSSDHIPSDYDDVGSNSGDISNNYDDVGSNNGDISSNYDDAAADYEPIRTTENIYESIGGSRTSGPENTSGGAAAALNSLRGSSYSNYDDAAADYEPIRTTENIYESIGGSRTSGPENTSGGAAAALNSLRGSSYSNYDDAAADYEPIRTTENIYESIGGSRTSGPENTSDGAAAAALNSLRGSSYTTGPRNEGVFGPGPEGLPDMSLPSYDPTNKTSLLTFLSNPHVKSKMLENSGHFVFIDTDRSSFILVPNGNWDQVCSIKVQNGKTKEDLDIKDLENMCAKFCTGFSKFSGDWDSLVEPMVSAKAGVASGGNLPNTVIINNKFKTCVAYGPWNSQEASSGYTPSAWRRGHRVDFGGIFEKANDFNKINWGTQAGPSSEDDGISFSNETPGAGPAAAPSPTPSSIPIINVNVNVGGTNVNIGDTNVNTTNTTPTTQSTDASTDTSDIDDINTNNQTDDINTTDKDSDGAGGVNGDISETESSSGDDSGSVSSSESDKNASVGNDGPAMKDILSAVRKHLDVVYPGENGGSTEGPLPANQTLGDVISDVENKGSAQDTKLSGNTGAGDDDPTTTAAVGNGAEEITLSDTDSGIGDDVSDTASSSGDESGGVSSPSSESNKNTAVGNDGPSGLDILAAVRKHLDKVYPGDNGGSTEGPLQANQTLGDIVQDMETTGTSQETVVSPWKGSTSSTESAGGSGSVQTLLPSPPPTPSTTTLRTGTGATTTSLMMGGPIKADIITTGGGGRIPGGGTLEKLLPRIRAHLDISFDAQGDLVSTEEPQLGSIVNKFRQETGSRGILAFVESAPGKPGSAQVLTGTGGDKGNLFQAAAAVTQALGNVAGKVNLAIQGQKLSSLVNDDGKGSVGRDLFQAAAQTTQVLSALIDTVG

SEQ ID NO: 62 - protein, 9 aa, Chlamydia trachomatis
YVFDRILKV

SEQ ID NO: 63 - protein, 9 aa, Chlamydia trachomatis
GLTEEIDYV

SEQ ID NO: 64 - protein, 9 aa, Chlamydia trachomatis
YMDNNLFYV

SEQ ID NO: 65 - protein, 9 aa, Chlamydia trachomatis
FLTLAWWFI

SEQ ID NO: 66 - protein, 119 aa, Chlamydia trachomatis
MSKKHKHKQAHTSSKPKVEPAYVSKKESPALQELQNAMISFSQDLPLAQMFSEIQDEKQLAKMMAALSGMLDSLPVETLTKGVFDNPKEEAQLSQEISSIFLGLKHLTETVNKHIADEK

SEQ ID NO: 67 - protein, 354 aa, Chlamydia trachomatis
MSQSTYSLEQLADFLKVEFQGNGATLLSGVEEIEEAKTAHITFLDNEKYAKHLKSSEAGAIIISRTQFQKYRDLNKNFLITSESPSLVFQKCLELFITPVDSGFPGIHPTAVIHPTAIIEDHVCIEPYAVVCQHAHVGSACHIGSGSVIGAYSTVGEHSYIHPRVVIRERVSIGKRVIIQPGAVIGSCGFGYVTSAFGQHKHLKHLGKVIIEDDVEIGANTTIDRGRFKHSVVREGSKIDNLVQIAHQVEVGQHSMIVAQAGIAGSTKIGNHVIIGGQAGITGHICIADHVIMMAQTGVTKSITSPGIYGGAPARPYQEIHRQVAKVRNLPRLEERIAALEKLVQKLEALSEQH

SEQ ID NO: 68 - protein, 196 aa, Chlamydia trachomatis
MFKLLLIFADPAEAARTLSLFPFSLNKENFYTYHTENVLLDVMVLKTWGYRGVVQALSPPPSGYDLWINAGFAGAGNPNIPLLKTYTITSVKELTPTTSVEEELEVTPIPRLPLAQLTSVRSPYRDGFHEHLQLVDMEGFFIAKQASLVACPCSMIKVSSDYTTREGQDFLKNNKVKLSQKLAEAIFPIYSSFIDV

SEQ ID NO: 69 - protein, 9 aa, Chlamydia trachomatis
KLAEAIFPI

SEQ ID NO: 70 - protein, 9 aa, Chlamydia trachomatis
FLKNNKVKL

SEQ ID NO: 71 - protein, 9 aa, Chlamydia trachomatis
ALSPPPSGY

SEQ ID NO: 72 - protein, 9 aa, Chlamydia trachomatis
FIAKQASLV

SEQ ID NO: 73 - protein, 9 aa, Chlamydia trachomatis
TLSLFPFSL

SEQ ID NO: 74 - protein, 9 aa, Chlamydia trachomatis
SLVACPCSM

SEQ ID NO: 75 - protein, 9 aa, Chlamydia trachomatis
LIFADPAEA

SEQ ID NO: 76 - protein, 9 aa, Chlamydia trachomatis
LLLIFADPA

SEQ ID NO: 77 - protein, 9 aa, Chlamydia trachomatis
RLEEVSQKL

SEQ ID NO: 78 - protein, 9 aa, Chlamydia trachomatis
LTTDTPPVL

SEQ ID NO: 79 - protein, 9 aa, Chlamydia trachomatis
KLLDMEGYA

SEQ ID NO: 80 - protein, 9 aa, Chlamydia trachomatis
VLSEDPPYI

SEQ ID NO: 81 - protein, 9 aa, Chlamydia trachomatis
ALQSYCQAY

SEQ ID NO: 82 - protein, 9 aa, Chlamydia trachomatis
KLTQTLVEL

SEQ ID NO: 83 - protein, 9 aa, Chlamydia trachomatis
FVGACSPEI

SEQ ID NO: 84 - protein, 9 aa, Chlamydia trachomatis
NLTTDTPPV

SEQ ID NO: 85 - protein, 9 aa, Chlamydia trachomatis
LMERAIPPK

SEQ ID NO: 86 - protein, 324 aa, Chlamydia trachomatis
MELLPHEKQVVEYEKTIAEFKEKNKENSLLSSSEIQKLDKRLDRLKEKIYSDLTPWERVQICRHPSRPRTVNYIEGMCEEFVELCGDRTFRDDPAVVGGFAKIQGQRFMLIGQEKGCDTKSRMHRNFGMLCPEGFRKALRLAKMAEKFGLPIIFLVDTPGAFPGLTAEERGQGWAIATNLFELARLATPIIVIVIGEGCSGGALGMAIGDVVAMLEHSYYSVISPEGCASILWKDPKKNSDAAAMLKMHGEDLKGFAIVDAVIKEPIGGAHHNPAATYRSVQEYVLQEWLKLKDLPVEELLEKRYQKFRTIGLYETSSESDSEA

SEQ ID NO: 87 - protein, 854 aa, Chlamydia trachomatis
MFEKFTNRAKQVIKLAKKEAQRLNHNYLGTEHILLGLLKLGQGVAVNVLRTLGVDFDTAKHEVERLIGYGPEIQVCGDPALTGRVKKSFESANEEAALLEHNYVGTEHLLLGILNQSDGVALQVLENLHVDPKEIRKEILKELETFNLQLPPSSSITPRNTNSSSSSKSSSPLGGHTLGGDKPEKLSALKAYGYDLTEMFKESRLDPVIGRSAEVERLILILCRRRKNNPVLVGEAGVGKTAIVEGLAQKIVSGEVPEALRKKRLITLDLALMIAGTKYRGQFEERIKAVMDEVRKHGNILLFIDELHTIVGAGAAEGAIDASHILKPALARGEIQCIGATTLDEYRKHIEKDAALERRFQKIVVQPPSVDETVEILRGLKKKYEEHHNVFITDEALVAAAKLSDQYVHGRFLPDKAIDLLDEAGARVRVNTMGQPSDLVRLEAEIEKTKQAKEQAIGTQEYEKAASLRDEEKKLREKLGNMKQQWESNKEEHQVPVDEEAVAQVVSVQTGIPAARLTEAESEKLLTLETTLQKKVIGQSQAVASICRAIRRSRTGIKDPNRPMGSFLFLGPTGVGKTLLAQQIAIEMFGGEDSLIQVDMSEYMEKFAATKMMGSPPGYVGHEEGGHLTEQVRRRPYCVVLFDEIEKAHPDIMDLMLQILEQGRLTDSFGRKIDFRNTIIIMTSNLGADLIRKSGEIGFGLRSHMDYAVIKEKIDAAVKKHLKPEFINRLDESVIFKPLEKEALSEIIHLEINKLGSRLQNYQMDLNIPDSVISFLVTKGHSPEMGARPLRRVVEQYLEDPLAEMLLKESCRQEARKLRARLTEERVVFEREEEAVSALAIEGDGSEPITADES

SEQ ID NO: 88 - protein, 145 aa, Chlamydia trachomatis
MKFFCKLESGSSLPEYATSGASGADVRANINEPIAILPGQRALIPTGISVEIPHGYEIQVRSRSGLASKYGVIVLQSPGTVDADYRGEIRVILANLGEATFIVEPGMRIAQLVVAKVEQVSFVETQEELTATARGTGGFGHTGEC

SEQ ID NO: 89 - protein, 124 aa, Chlamydia trachomatis
MPLTDEEIANFKTRLLEMKAKLSHTLEGNAQEVKKPNEATGYSQHQADQGTDTFDRTISLEVTTKEYKLLRQIDRALEKIEEASYGICDVSGEEIPLARLMAIPYATMTVKSQEKFEKGLLSGN

SEQ ID NO: 90 - protein, 183 aa, Chlamydia trachomatis
MECLQQDTGVEAEQVQVQQQEENAVPVTSQRVSITQAAKLHNVTRQAIYVAIKQKKLKASKTTRWEIDLQDLEDYRRNRYSRAKSTYQGELLFDNEKGFYSVGQVASMLDVPEQKIYYATRIGAMKGERRGSAWVIHVSEVDRYRNDYLKKEAERKGKSLAAMREGFEALGADLLADAENFIS

SEQ ID NO: 91 - protein, 86 aa, Chlamydia trachomatis
MSQNKNSAFMQPVNVSADLAAIVGAGPMPRTEIIKKMWDYIKKNGLQDPTNKRNINPDDKLAKVFGTEKPIDMFQMTKMVSQHIIK

SEQ ID NO: 92 - protein, 439 aa, Chlamydia trachomatis
MTTGVRGDNAPDPSLLAQLTQNANSASAASTGKNGQVAGAKQENVDASFEDLLQDAQGTGGSKKATANQTSKSGKSEKAQASSGTSTTTSVAQASQTATAQAVHGARDSGFNSDGSATLPSPTGTEVNGVVLRKGMGTLALMGLIMTLLAQASAKSWSSSFQQQNQAIQNQVAMAPEIGNAIRTQANHQAQATELQAQQSLISGITNIVGFAVSVGGGILSASKSLGGLKSAAFTNETASATTSATSSLAKTATSALDDVAGTATAVGAKATSGAASAASSAATKLTQNMAESASKTLSQTASKSAGGLFGQALNTPSWSEKVSRGMNVVKTQGTRAAKFAGRALSSAMNISQMVHGLTAGIDGIVGGVIGAQVAQEQRMAGMAEARAEELKSLNSVQAQYASQAQQLQEQSQQSFNSALQTLQSISDSALQTTASMFN

SEQ ID NO: 93 - protein, 231 aa, Chlamydia trachomatis
MMEVFMNFLDQLDLIIQNKHMLEHTFYVKWSKGELTKEQLQAYAKDYYLHIKAFPKYLSAIHSRCDDLEARKLLLDNLMDEENGYPNHIDLWKQFVFALGVTPEELEAHEPSEAAKAKVATFMRWCTGDSLAAGVAALYSYESQIPRIAREKIRGLTEYFGFSNPEDYAYFTEHEEADVRHAREEKALIEMLLKDDADKVLEASQEVTQSLYGFLDSFLDPGTCCSCHQSY

SEQ ID NO: 94 - protein, 352 aa, Chlamydia trachomatis
MSVPDRKRALEAAIAYIEKQFGAGSIMSLGKHSSAHEISTIKTGALSLDLALGIGGVPKGRIVEIFGPESSGKTTLATHIVANAQKMGGVAAYIDAEHALDPNYAALIGANINDLMISQPDCGEDALSIAELLARSGAVDVIVIDSVAALVPKSELEGEIGDVHVGLQARMMSQALRKLTATLARTNTCAIFINQIREKIGVSFGNPETTTGGRALKFYSSIRIDIRRIGSIKGGENFDIGNRIKVKVAKNKLAPPFRTAEFDILFNEGISSAGCIIDLAVEKNIIDKKGSWFNYQDRKLGQGREAVREELKRNKELFHELERRIYESVQASQVPAAACVDAESREVAEAAK

SEQ ID NO: 95 - protein, 269 aa, Chlamydia trachomatis
MFPENKMLLIAGPCVIEDNSVFETARRLKEIVAPYASSVHWIFKSSYDKANRSSVHNYRGPGLKLGLQTLAKIKEELDVEILTDVHSPDEAREAAKVCDIIQVPAFLCRQTDLLVTAGETQAIVNIKKGQFLSPWEMQGPIDKVLSTGNNKIILTERGCSFGYNNLVSDMRSIEVLRRFGFPVVFDGTHSVQLPGALHSQSGGQTEFIPVLTRSAIAAGVQGLFIETHPNPSSALSDAASMLSLKDLERLLPAWVQLFTYIQEMDAVSV

SEQ ID NO: 96 - protein, 223 aa, Chlamydia trachomatis
MIDPLKLFPNFDGDKESAAVNKPSASPMPSELSKNVASFSLGGGGAALDSTVSTEKLSLMAMMQDKNSQLIDPELEEALNSEELQEQIHLLKSRLWDAQTQMQMQDPDKLASEHVDALGVIVDLINGDFQAIAEHTQQTVKQGNGDEEKSVTRKIVDWVSSGEEILNRALLYFSDRNGERETLADFLKVQYAVQRATQRAELFASILGATVSSVKTIMTTQLG

SEQ ID NO: 97 - protein, 224 aa, Chlamydia trachomatis
MQVLASLFGQSPFAPLQAHLELVSSTINVLFPLFSALKEGDYERVGVLAQLVSSKERQADGMKNDVRRHLASGVFLPVSRAALLEIISIQDSLADCAEDIAILLTVKELQFYPEFEELFFEFLQKTVQSFEAVAKTIREMDRLLESSFGGNRADKTRVLVSEVSNLEHECDLLQRELMKVFFSDDFAIGTKGFVLWMQIIKGISGISNNSEKLAYRVSMTLEEK

SEQ ID NO: 98 - protein, 9 aa, Chlamydia trachomatis
LLQRELMKV

SEQ ID NO: 99 - protein, 9 aa, Chlamydia trachomatis
STINVLFPL

SEQ ID NO: 100 - protein, 9 aa, Chlamydia trachomatis
PLQAHLELV

SEQ ID NO: 101 - protein, 9 aa, Chlamydia trachomatis
SLFGQSPFA

SEQ ID NO: 102 - protein, 9 aa, Chlamydia trachomatis
KLAYRVSMT

SEQ ID NO: 103 - protein, 9 aa, Chlamydia trachomatis
VLWMQIIKG

SEQ ID NO: 104 - protein, 9 aa, Chlamydia trachomatis
VLFPLFSAL

SEQ ID NO: 105 - protein, 9 aa, Chlamydia trachomatis
FLQKTVQSF

SEQ ID NO: 106 - protein, 9 aa, Chlamydia trachomatis
FGQSPFAPL

SEQ ID NO: 107 - protein, 9 aa, Chlamydia trachomatis
YMLPIFTAL

SEQ ID NO: 108 - protein, 9 aa, Chlamydia trachomatis
LLHEFNQLL

SEQ ID NO: 109 - protein, 9 aa, Chlamydia trachomatis
VLQRELMQI

SEQ ID NO: 110 - protein, 9 aa, Chlamydia trachomatis
PLQAHLEMV

SEQ ID NO: 111 - protein, 9 aa, Chlamydia trachomatis
RLFGQSPFA

SEQ ID NO: 112 - protein, 9 aa, Chlamydia trachomatis
GLFMPISRA

SEQ ID NO: 113 - protein, 9 aa, Chlamydia trachomatis
KLAHRINMT

SEQ ID NO: 114 - protein, 9 aa, Chlamydia trachomatis
YLWLQVIRR

SEQ ID NO: 115 - protein, 9 aa, Chlamydia trachomatis
TLLHEFNQL

SEQ ID NO: 116 - protein, 9 aa, Chlamydia trachomatis
FGQSPFAPL

SEQ ID NO: 117 - protein, 221 aa, Chlamydia trachomatis
MKKFIYKYSFGALLLLSGLSGLSSCCANSYGSTLAKNTAEIKEESVTLREKPDAGCKKKSSCYLRKFFSRKKPKEKTEPVLPNFKSYADPMTDSERKDLSFVVSAAADKSSIALAMAQGEIKGALSRIREIHPLALLQALAEDPALIAGMKKMQGRDWVWNIFITELSKVFSQAASLGAFSVADVAAFASTLGLDSGTVTSIVDGERWAELIDVVIQNPAI

SEQ ID NO: 118 - protein, 349 aa, Chlamydia trachomatis
MSSLRIARSCSFKQKTLLAALVFFGLGASQVCATPSNSREIAWYVDYQEARDESREKDSPMLLFFSGSDWNGSCMKIRDEVLSSSDFISAVADQFVCVVVDFPRHTELRDPLINEQNEDLKNRLHVNTFPSLVLLSPEERAIYKIESFGNENGSNLGESLCRVIANDQELEQVFPLIPTLSSVELRKYYQLAEELSRKDFMATALEQGVLCDDSFFLSEKFRQLVEAGRMDSEECRAVKNRLLELDPENEQFTHFTVALIEFQELAKRSQGQSHVNVAEVIAPLAEYLTQFGEQDKENQWRLEMIISQYYLDAGLPNSALEHAKIAFESAPQDIQSSISRSLVHIEHQS

SEQ ID NO: 119 - protein, 601 aa, Chlamydia trachomatis
MKMNRIWLLLLTFSSAIHSPVQGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 120 - protein, 9 aa, Chlamydia trachomatis
VLADFIGGL

SEQ ID NO: 121 - protein, 9 aa, Chlamydia trachomatis
RMASLGHKV

SEQ ID NO: 122 - protein, 9 aa, Chlamydia trachomatis
GLNDFHAGV

SEQ ID NO: 123 - protein, 9 aa, Chlamydia trachomatis
FSCADFFPV

SEQ ID NO: 124 - protein, 9 aa, Chlamydia trachomatis
MLTDRPLEL

SEQ ID NO: 125 - protein, 9 aa, Chlamydia trachomatis
LLENVDTNV

SEQ ID NO: 126 - protein, 9 aa, Chlamydia trachomatis
RMILTQDEV

SEQ ID NO: 127 - protein, 9 aa, Chlamydia trachomatis
SCADFFPVV

SEQ ID NO: 128 - protein, 9 aa, Chlamydia trachomatis
FVFNVQFPN

SEQ ID NO: 129 - protein, 9 aa, Chlamydia trachomatis
YLYALLSML

SEQ ID NO: 130 - protein, 9 aa, Chlamydia trachomatis
SLAVREHGA

SEQ ID NO: 131 - protein, 9 aa, Chlamydia trachomatis
YLPYTVQKS

SEQ ID NO: 132 - protein, 9 aa, Chlamydia trachomatis
ATIAPSIRA

SEQ ID NO: 133 - protein, 9 aa, Chlamydia trachomatis
LLEVDGAPV

SEQ ID NO: 134 - protein, 9 aa, Chlamydia trachomatis
RTAGAGGFV

SEQ ID NO: 135 - protein, 9 aa, Chlamydia trachomatis
SLFYSPMVP

SEQ ID NO: 136 - protein, 591 aa, Chlamydia trachomatis
MSIRGVGGNGNSRIPSHNGDGSNRRSQNTKGNNKVEDRVCSLYSSRSNENRESPYAVVDVSSMIESTPTSGETTRASRGVFSRFQRGLVRVADKVRRAVQCAWSSVSTRRSSATRAAESGSSSRTARGASSGYREYSPSAARGLRLMFTDFWRTRVLRQTSPMAGVFGNLDVNEARLMAAYTSECADHLEANKLAGPDGVAAAREIAKRWEQRVRDLQDKGAARKLLNDPLGRRTPNYQSKNPGEYTVGNSMFYDGPQVANLQNVDTGFWLDMSNLSDVVLSREIQTGLRARATLEESMPMLENLEERFRRLQETCDAARTEIEESGWTRESASRMEGDEAQGPSRAQQAFQSFVNECNSIEFSFGSFGEHVRVLCARVSRGLAAAGEAIRRCFSCCKGSTHRYAPRDDLSPEGASLAETLARFADDMGIERGADGTYDIPLVDDWRRGVPSIEGEGSDSIYEIMMPIYEVMDMDLETRRSFAVQQGHYQDPRASDYDLPRASDYDLPRSPYPTPPLPPRYQLQNMDVEAGFREAVYASFVAGMYNYVVTQPQERIPNSQQVEGILRDMLTNGSQTFRDLMRRWNREVDRE

SEQ ID NO: 137 - protein, 218 aa, Chlamydia trachomatis
MTASDGISLTVSNNSSTNASITIGLDAEKAYQLILEKLGNQILDGIADTIVDSTVQDILDKITTDPSLGLLKAFNNFPITNKIQCNGLFTPSNIETLLGGTEIGKFTVTPKSSGSMFLVSADIIASRMEGGVVLALVREGDSNPCAISYGYSSGVPNLCSLRTSITNTGLTPTTYSLRVGGLESGVVWVNALSNGNDILGITNTSNVSFLEVIPQTNA

SEQ ID NO: 138 - protein, 67 aa, Chlamydia trachomatis
MQHTIMLSLENDNDKLASMMDRVVAASSSILSASKDSESNRQFTISKAPDKEAPCRVSYVAASALSE

SEQ ID NO: 139 - protein, 442 aa, Chlamydia trachomatis
MEEITTEFNTLMTELPDVQLTAVVGRIIEVVGMLIKAVVPDVRVGEVCLVKRHGMEPLVTEVVGFTQNFVFLSPLGELTGVSPSSEVMATGLPLHIRAGEGLLGRVLNGLGNPIDTETKGPLENVDAIYPIFKAPPDPLHRAKLRTILSTGVRCIDGMLTVAKGQRIGIFAGAGVGKSSLLGMIARNAEEADINVIALIGERGREVREFIENDLGEEGMKRSVIVVSTSDQSSQLRLNAAYVGTAIAEYFRDQGKTVVLMMDSVTRFARALREVGLAAGEPPARAGYTPSVFSTLPKLLERAGASDKGTITAFYTVLVAGDDMNEPVADEVKSILDGHIVLSNALAQAYHYPAIDVLASISRLLTAIVPEEQRRIIGRAREVLAKYKANEMLIRIGEYRRGSDREVDFAIDHIDKLNRFLKQDIHEKTNYEEAAQQLRAIFR

SEQ ID NO: 140 - protein, 223 aa, Chlamydia trachomatis
MKFFSLIYKDQEVVPNKKVLSPDAYTAVLTAQELLEKTQEDCEAYTQNTHEECAKLREEAKNQGFQEGSKAWSKQLAFLITETQAMREQIKASLVPLAIASIKKIIGKELETKPETVVSIISESLKDLTQNKRIVIHINPQDLAIVEQHRPELKKLVEYADVLLLSPKASVSPGGCIIETETGIVNAQLDVQLAALEQAFSAILKHKKPADASTIDQPQSKKD

SEQ ID NO: 141 - protein, 146 aa, Chlamydia trachomatis
MQNQFEQLLTELGTQINSPLTPDSNNACIVRFGYNNVAVQIEEDGNSGFLVAGVMLGKLPENTFRQKIFKAALSINGSPQSNIKGTLGYGEISNQLYLCDRLNMTYLNGEKLARYLVLFSQHANIWMQSISKGALPDLHALGMYHL

SEQ ID NO: 142 - protein, 278 aa, Chlamydia trachomatis
MTANTFGILNILMKQAKADDLAQFLPEHLLLDSPHHQDIPLQSLSFNMRWLATIHPSWISVAMKEFPPVVQSQLLAWLPLPLTQELLPLLDSGVTPATKRCLDFGAFYLLDLLSKKVRPPGITEEIFLPASPFNAMLYYVGPTKMALINCLGLYTLAQEMRNVVDRVVIDRVQRVLSETERMFLNYCKTHPMKHLEPMAFLASWEEDQALRHFIHVQGLRFLARALAKEDSSFLWYFIRRLDVGRGYIFEKALQSSIDSPHNEYFRERLEHCISILVQ

SEQ ID NO: 143 - protein, 315 aa, Chlamydia trachomatis
MKKFATFLCVLLSGSGFAAPVEVPGFPSIPETYITINDKELGLQEHCRGVNVLSCGYNLVGMFHTPTTPMPLGGYPTVIFFHGFRGNCTGKDGVYRDLARLLTANGIAVARFDMAGCGNSEGICDQIPARTYLRNGEDILATVAKYPEVNPHRIGIAGISLGCHTTIHLASTYRPRDYTVQAISVWAPIADGVILLKEICATIGLTMTQFSDMGEVGKAFGFKQLPLKLCRDDIDFFLGIQDHILLLSLPRRIPVLHQQGLEDHVVSTAHQRLFLGAAPAQMLSKSYPETPHEIASSPYRQEVLQEILTHFQSNL

SEQ ID NO: 144 - protein, 9 aa, Chlamydia trachomatis
FLGAAPAQM

SEQ ID NO: 145 - protein, 9 aa, Chlamydia trachomatis
FLGIQDHIL

SEQ ID NO: 146 - protein, 9 aa, Chlamydia trachomatis
LLTANGIAV

SEQ ID NO: 147 - protein, 9 aa, Chlamydia trachomatis
SLPRRIPVL

SEQ ID NO: 148 - protein, 9 aa, Chlamydia trachomatis
GLQEHCRGV

SEQ ID NO: 149 - protein, 9 aa, Chlamydia trachomatis
SLGCHTTIH

SEQ ID NO: 150 - protein, 9 aa, Chlamydia trachomatis
ILTHFQSNL

SEQ ID NO: 151 - protein, 9 aa, Chlamydia trachomatis
VLSCGYNLV

SEQ ID NO: 152 - protein, 9 aa, Chlamydia trachomatis
LLKEICATI

SEQ ID NO: 153 - protein, 9 aa, Chlamydia trachomatis
RLFLGAAPA

SEQ ID NO: 154 - protein, 9 aa, Chlamydia trachomatis
ATVAKYPEV

SEQ ID NO: 155 - protein, 9 aa, Chlamydia trachomatis
LLSGSGFAA

SEQ ID NO: 156 - protein, 9 aa, Chlamydia trachomatis
LTANGIAVA

SEQ ID NO: 157 - protein, 9 aa, Chlamydia trachomatis
SGFAAPVEV

SEQ ID NO: 158 - protein, 141 aa, Chlamydia trachomatis
MEQTLSIIKPDSVGKAHIGEIIAIFEKSGLRIAAMKMVHLSVKEAEGFYVVHKERPFFQELVDFMISGPVVVMVLQGENAVDRNRELMGATNPKEAAEGSIRALFGESIGVNAVHGSDSLENAAIEVSYFFAKTEIVNSVA

SEQ ID NO: 159 - protein, 9 aa, Chlamydia trachomatis
FMISGPVVV

SEQ ID NO: 160 - protein, 9 aa, Chlamydia trachomatis
ALFGESIGV

SEQ ID NO: 161 - protein, 9 aa, Chlamydia trachomatis
SLENAAIEV

SEQ ID NO: 162 - protein, 9 aa, Chlamydia trachomatis
LMGATNPKE

SEQ ID NO: 163 - protein, 9 aa, Chlamydia trachomatis
RIAAMKMVH

SEQ ID NO: 164 - protein, 913 aa, Chlamydia trachomatis
MAKDKKTNPESKKSFPTAFFFLLFGVIFGVVTVQNFFSAKKASVGFSHQLEHLVNLKLLIPEESRKTALNDNLVSFSGRFREVVPAEGQVRYQYLDLIERKHQIDFELEEASKSLTVLSKEVRNAITWFSAISGMPIPEAGYTISPRTDVGLSVLEPLVVYGPVDAQIVNLAALENRVRSLPKSTESLRVFGSDLYALIGKYLSPALGIGSESLKKEIKDLHQQVENSLTQVIEGDQAVALYKTVLETLHRISLALVSPEEGTRFHQLRSVRLYREDFNRCVKLLGESDETQVQLDKLRGELVQAVWYFNNQELSSRALEKQDPEVFSRWFEGAKQEWAAFSSNKSLSFRAPDQPRNLVLEKTFRSEEPTPHYSGYLFTFMPIILVLLFIYFIFSRQVKGMNGSAMSFGKSPARLLAKGQNKVTFADVAGIEEAKEELVEIVDFLKNPTKFTSLGGRIPKGILLIGAPGTGKTLIAKAVAGEADRPFFSIAGSDFVEMFVGVGASRIRDMFEQAKRNAPCIIFIDEIDAVGRHRGAGIGGGHDEREQTLNQLLVEMDGFGTNEGVILMAATNRPDVLDKALLRPGRFDRRVVVNLPDIKGRFEILAVHAKRIKLDPTVDLMAVARSTPGASGADLENLLNEAALLAARKDRTAVTAVEVAEARDKVLYGKERRSLEMDAQEKKTTAYHESGHAIVGLCVEHSDPVDKVTIIPRGLSLGATHFLPEKNKLSYWKKELYDQLAVLMGGRAAEQIFLGDVSSGAQQDIAQATKIVRSMICEWGMSDHLGTVAYDERSEAAPTGYGSCHEKNYSEETAKAIDNELKTLLDAAYQRALDIINSHKEELELMTQMLIEFETLDSKDVKEIMDHSWDADKKQARMKEEGMLYKKISEDLPPPPPQENVQDGTSLKFNTST

SEQ ID NO: 165 - protein, 9 aa, Chlamydia trachomatis
LLFGVIFGV

SEQ ID NO: 166 - protein, 9 aa, Chlamydia trachomatis
LLAKGQNKV

SEQ ID NO: 167 - protein, 9 aa, Chlamydia trachomatis
FTFMPIILV

SEQ ID NO: 168 - protein, 9 aa, Chlamydia trachomatis
FLGDVSSGA

SEQ ID NO: 169 - protein, 9 aa, Chlamydia trachomatis
LLDAAYQRA

SEQ ID NO: 170 - protein, 9 aa, Chlamydia trachomatis
GMSDHLGTV

SEQ ID NO: 171 - protein, 9 aa, Chlamydia trachomatis
SLGATHFLP

SEQ ID NO: 172 - protein, 9 aa, Chlamydia trachomatis
NLAALENRV

SEQ ID NO: 173 - protein, 9 aa, Chlamydia trachomatis
YLFTFMPII

SEQ ID NO: 174 - protein, 9 aa, Chlamydia trachomatis
FPTAFFFLL

SEQ ID NO: 175 - protein, 9 aa, Chlamydia trachomatis
ILMAATNRP

SEQ ID NO: 176 - protein, 9 aa, Chlamydia trachomatis
KTALNDNLV

SEQ ID NO: 177 - protein, 9 aa, Chlamydia trachomatis
LLNEAALLA

SEQ ID NO: 178 - protein, 9 aa, Chlamydia trachomatis
ELYDQLAVL

SEQ ID NO: 179 - protein, 9 aa, Chlamydia trachomatis
ALEKQDPEV

SEQ ID NO: 180 - protein, 9 aa, Chlamydia trachomatis
SLGGRIPKG

SEQ ID NO: 181 - protein, 9 aa, Chlamydia trachomatis
FMPIILVLL

SEQ ID NO: 182 - protein, 9 aa, Chlamydia trachomatis
LLAARKDRT

SEQ ID NO: 183 - protein, 9 aa, Chlamydia trachomatis
VTFADVAGI

SEQ ID NO: 184 - protein, 9 aa, Chlamydia trachomatis
YTISPRTDV

SEQ ID NO: 185 - protein, 9 aa, Chlamydia trachomatis
LIGAPGTGK

SEQ ID NO: 186 - protein, 157 aa, Chlamydia trachomatis
MLFGYLVGFLAADPEERMTSGGKRVVVLRLGVKSRVGSKDETVWCRCNIWNNRYDKMLPYLKKGSSVIVAGELSLESYVGRDGSPQASISVSVDTLKFNSGSSRPDARGSDEGRQRANDNVSIGFDGESLDTDSALDKEVYAGFGEDQQYASEDVPF